(19) United States
(12) Patent Application Publication
(10) Pub. No.: US 2009/0068290 A1
(43) Pub. Date: Mar. 12, 2009

(54) BIFEPRUNOX DOSES FOR TREATING SCHIZOPHRENIA

(76) Inventors: Michel Bourin, Nantes (FR); Daniel E. Casey, Portland, OR (US); Steven G. Potkin, Irvine, CA (US); Mark Rapaport, Los Angeles, CA (US); John Newcomer, St. Louis, MO (US); Paul P. Yeung, Southeastern, PA (US); Sangeeta Raje, Collegeville, PA (US); Jeff Paul, Collegeville, PA (US); Saeed Ahmed, Collegeville, PA (US); Luigi M. Barbato, Alpharetta, GA (US); Nathan A. Shapira, Atlanta, GA (US); Roseline Pardue, Marietta, GA (US); Antje A. Winsemius, Weesp (NL); Michiel H. de Vries, Weesp (NL); Marc Debelle, Valby-Copenhagen (DK); Jens Heisterberg, Valby-Copenhagen (DK); Mette Krog Josiassen, Valby-Copenhagen (DK); Jette Buch Ostergard, Valby-Copenhagen (DK); Dorte Malling, Valby-Copenhagen (DK); Ellen B. Christensen, Valby-Copenhagen (DK)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 11/847,458

(22) Filed: Aug. 30, 2007

Related U.S. Application Data
(60) Provisional application No. 60/841,244, filed on Aug. 31, 2006.
(60) Provisional application No. 60/841,495, filed on Sep. 1, 2006.
(60) Provisional application No. 60/878,693, filed on Jan. 5, 2007.

(51) Int. Cl. A61K 31/496; A61K 33/00; A61K 31/4525; A61K 31/4196

(52) U.S. Cl. 424/722; 514/254.02; 514/321; 514/383; 514/254.07

(57) ABSTRACT

The present disclosure is directed to compositions, kits and methods for not only treating schizophrenia, e.g., maintaining, reducing and/or improving at least one symptom of schizophrenia, but also to achieving an absence of or a reduced presence of at least one side effect associated with schizophrenia therapy with a first-generation atypical antipsychotic.

This application claims priority to U.S. Provisional Application No. 60/841,244, filed on Aug. 31, 2006, U.S. Provisional Application No. 60/841,495, filed on Sep. 1, 2006, and U.S. Provisional Patent Application No. 60/878,693, filed Jan. 2, 2007, all of which are incorporated herein by reference in their entirety.

The present invention relates to compositions, kits and methods for not only treating schizophrenia, e.g., maintaining, reducing and/or improving at least one symptom of schizophrenia, but also to achieving an absence of or a reduced presence of at least one side effect associated with schizophrenia therapy with a first-generation atypical antipsychotic. For example, the present invention relates to a composition for the treatment of schizophrenia, comprising a dose of at least one bifeprunox compound, which composition, when administered, over a treatment period, is effective to achieve treatment of at least one symptom of schizophrenia and an absence or a reduced presence of at least one side effect associated schizophrenia treatment with a first generation atypical antipsychotic.

Schizophrenia is a lifelong disabling psychiatric disorder characterized by severe and variable symptoms, including positive (i.e., hallucinations, delusions, racing thoughts) and negative symptoms (i.e., apathy, lack of emotion, poor or non-existent social functioning), cognitive deficits, and depression. The course of the illness can be divided into 4 major phases: (1) premorbid; (2) acute; (3) stable/maintenance; and (4) late course. The premorbid phase refers to symptoms, which occur before the onset of positive symptoms. During the acute phase, patients experience overt positive symptoms, such as delusions and hallucinations. The stable/maintenance phase may be divided into two sub-phases. The first 5 to 10 years of illness are often characterized by multiple exacerbations of positive symptoms, with more stable periods interspersed between acute episodes. This sub-phase is followed by a plateau phase, which is characterized by a stabilization of symptoms and a reduced number of exacerbations. Key treatment goals during the maintenance phase are to facilitate the patient's return to the community and establish a long-term maintenance plan. In the late course phase of the illness, positive symptoms tend to diminish with age and many patients with long-term impairments regain some degree of social and occupational competence; however, the effects of years of dysfunction are rarely overcome.

In the maintenance and late course phases of schizophrenia therapy, the majority of patients continue to face a number of problems, such as a need for further symptomatic improvement and long-term control of psychotic symptoms, including prevention of relapses; maintenance of cognitive function; prevention of weight increase, hyperglycemia, and dyslipidemia; and generally, improvement in quality of life. In addition, positive symptoms may become more resistant to treatment with each succeeding episode. Consistent with this notion, 85%-90% of patients with schizophrenia experience clinical deterioration. Further, at least half of patients given antipsychotic agents do not comply with the treatment regimen prescribed and thus, are at risk for relapse. Therefore, despite intense effort aimed at improving treatment, a large proportion of patients with schizophrenia are severely disabled; they relapse often and may require hospitalization.

Compounds currently used to treat schizophrenia, such as first-generation atypical antipsychotics that act as dopamine D2 receptor antagonists and, most of them, also as serotonin receptor antagonists, have been associated with several undesirable side effects, i.e., treatment induced side effects. First-generation atypical antipsychotics include, but are not limited to, aripirazole, amisulpride, clozapine, olanzapine, quetiapine, risperidone, ziprasidone, and zotepine. These treatment induced side effects include, among other things, weight gain, hyperprolactinemia, elevated triglyceride levels, metabolic syndrome (markers: diabetes, hyperlipidemia, hypertension, and obesity), prolonged OTc intervals, glucose abnormalities, and exhibition of extrapyramidal symptoms. For example, prolonged QTc intervals, i.e., the corrected OT interval in an electrocardiogram, can lead to problematic heart rhythms, or heart arrhythmias. Similarly, the weight gain observed with conventional atypical antipsychotics, such as risperidone and olanzapine, has been associated with an increased risk of cardiovascular disease and diabetes mellitus.

In addition, schizophrenia therapy generally may be over extended periods of time. As such, these treatment induced side effects of the therapy itself affect patients on a daily basis, as well as contributing to their long-term health. Moreover, these treatment induced side effects may also lead to noncompliance with a patient's treatment regimen. Even when treatment is for a limited and/or short duration, these treatment induced side effects affect a patient's willingness to comply with treatment regimens. Therefore, treatment compositions and methods are needed for maintaining, reducing, and/or improving these undesirable (i.e., treatment induced) side effects associated with first-generation atypical antipsychotics, as well as treating, e.g., maintaining, reducing, and/or improving baseline conditions of a patient undergoing schizophrenia treatment (i.e., control of schizophrenia symptoms).

The present inventors discovered that treating schizophrenia patients with at least one bifeprunox compound can maintain, reduce and/or improve at least one symptom of schizophrenia, while achieving an absence of or a reduced presence of at least one side-effect of schizophrenia therapy associated with a first generation atypical antipsychotic, i.e., the treatment induced side effects. In one embodiment of the disclosure, a composition comprises a therapeutically effective dose of at least one bifeprunox compound administered once-daily. Further embodiments include, for example, the dose of at least one bifeprunox compound ranging from about 1 mg to about 40 mg, or from about 5 mg to about 40 mg, such as about 20 mg to about 30 mg, and further for example, a dose of 20 mg or of 30 mg of at least one bifeprunox compound.

In maintaining, reducing and/or improving schizophrenia (such as the various symptoms of the disorder/disease), a therapeutically effective dose of the at least one bifeprunox compound or composition thereof can achieve reduction/improvement in at least one symptom of schizophrenia, and/or favorable effects, e.g., reduction/improvement in at least one efficacy measurement, such as, but not limited to, reduction of the Positive and Negative Syndrome Scale (PANSS) total score in a patient, maintenance or reduction of body weight, maintenance, reduction, and/or improvement of triglyceride levels and/or total cholesterol levels, maintenance of clinical stability of schizophrenia, such as in patients with chronic, stable schizophrenia, prevent deterioration of schizophrenia, improvement of one or more psychotic symptoms or maintenance and or/reduction of extrapyramidal signs and symptoms (EPS) profile, from a baseline measurement before administration. Other favorable effects are a reduction of the incidence of hyperglycemia and/or one or more diabetes-related adverse events. Thus, favorable effects suggest that the at least one bifeprunox compound or composition thereof can be effective for the treatment of schizophrenia.

In one embodiment of the disclosure, the at least one bifeprunox compound can be used for the long-term treatment of a patient with schizophrenia, such as in a daily dose ranging from about 20 to about 30 mg. As used herein, the term “long term treatment” refers, for example, to treatment lasting at least 3 months, at least 6 months, at least one year, or longer as necessitated by an evaluation of the patient.

The embodiments disclosed herein, while providing a general overview of various embodiments of the present disclosure, are not intended to limit the scope of the present disclosure in any manner.

U.S. patent application Ser. Nos. 10/920,361, 10/920,386, and 11/354,652 are hereby incorporated herein by reference in their entireties. All data herein are understood to be approximate and subject to normal measurement error depending, for example, on the apparatus used and other parameters influencing peak positions and peak intensities. Unless specifically defined otherwise or unless the context demands otherwise, the word “about” as used herein generally means ±5% of the recited value.

### BRIEF DESCRIPTION OF THE DRAWINGS

FIG. 1 is a graph of the PANSS Positive Score (FAS, LOCF).

FIG. 2 is a graph of the PANSS Negative Score (FAS, LOCF).

FIG. 3 is a graph of the PANSS General Psychopathology Score (FAS, LOCF).

FIG. 4 is a graph of the Proportion of Patients with at Least a 25% Reduction of PANSS Total Score (FAS, LOCF).

FIG. 5 is a graph of the Proportion of Patients with a CGI-I Score of 2 or Less (FAS, LOCF).

FIG. 6 is a graph of the percent of patients with ≧7% decrease in weight in short-term, placebo-controlled studies.

FIG. 7 is a graph of the percent of patients with ≧7% increase in weight in short-term placebo-controlled studies.

FIG. 8 is a graph of the percent of patients with ≧7% increase or decrease in weight.

Bifeprunox compounds are described and claimed in U.S. Pat. No. 6,225,312 and U.S. Pat. No. 7,030,241, the contents of which are incorporated herein by reference. The hydrochloric acid salt of this compound (7-[4-[(1,1′-biphenyl]-3-ylmethyl)-1-piperazinyl]-2(3H)-benzoxazolone (bifeprunox) is described and claimed in International Publication No. WO 97136893 and the monomethanesulfonate salt is described and claimed in International Publication No. WO 02/066449. In the second of these patent publications, the direct formation of the monomethanesulfonate salt by the reaction between the reactive mesylate ester of N,N,N-bis(2-ethanol)-m-phenylbenzyl amine and 7-amino-2(3H)-benzoxazolone is disclosed. A stable polymorph of bifeprunox monomethanesulfonate salt is disclosed and claimed in International Publication No. WO 05/016898. Also included in the term “bifeprunox compounds” are bifeprunox N-oxides. Bifeprunox N-oxide are disclosed and claimed in International Publication No. WO 07/023,141.

Bifeprunox compounds are indicated for the treatment of CNS (central nervous system) disorders, including schizophrenia, other psychotic disorders (for example, psychosis) and Parkinson's disease. In the framework of the present disclosure, dosage strength (or dose) is expressed in an amount equivalent to a bifeprunox base. As used herein, the term “bifeprunox base” refers to the compound 7-[4-([1,1′-biphenyl]-3-ylmethyl)-1-piperazinyl]-2(3H)-benzoxazolone (INN bifeprunox) having the following formula:

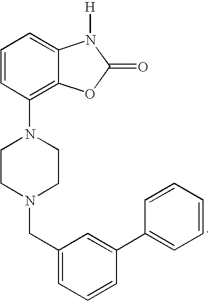

As used herein, the term “bifeprunox compound(s)” refers to the active compound 7-[4-([1,1′-biphenyl]-3-ylmethyl)-1-piperazinyl]-2(3H)-benzoxa-zolone, its N-oxide and pharmaceutically acceptable salts, solvates and hydrates thereof. When the N-oxide is used as the bifeprunox compound, the amount in milligrams is the same amount as the amount the person skilled in the art would select for the bifeprunox compound without the oxide. In addition, pharmaceutically acceptable salts of bifeprunox or its N-oxide may be obtained using standard procedures well known in the art, for example, by mixing a compound of the present invention with a suitable acid, for instance an inorganic acid or an organic acid.

The present disclosure is directed to compositions, kits, and methods for treating, i.e., maintaining, reducing and/or improving symptoms/conditions associated with schizophrenia (such as, at least one symptom of schizophrenia and/or at least one efficacy measurement) and to achieving an absence or a reduced presence of at least one side effect associated schizophrenia therapy with a first-generation atypical antipsychotic by administering to a patient in need thereof a therapeutically effective dose of at least one bifeprunox compound, or a composition thereof. For example, the dose of the at least one bifeprunox compound ranges from about 1 mg to about 40 mg or for example, from 5 mg to 40 mg, such as, from 10 mg to 40 mg, or further, for example, from 20 mg to 30 mg. In one embodiment, bifeprunox can be used in the treatment of a patient with schizophrenia having weight gain problems or susceptible to weight gain problems.

Other embodiments of the present disclosure relate to methods for preventing the deterioration of schizophrenia, reducing a patient's PANSS total score, and reducing a patient's triglyceride levels comprising administering to the patient a composition comprising a dose of at least one bifeprunox compound ranging from about 20 mg to about 30 mg over a treatment period, wherein the dose of the bifeprunox compound prevents deterioration of schizophrenia, reduces the patient's PANSS total score, and reduces the patient's triglyceride levels, respectively, from a baseline measurement before administration.

In a further embodiment the present disclosure relates to a method for treating an acutely exacerbated schizophrenic patient in need of treatment comprising: administering to the patient a composition comprising a dose of at least one bifeprunox compound ranging from about 20 mg to about 30 mg over a treatment period, wherein the composition is effective to improve at least one efficacy measurement in the patient, when compared with a baseline measurement before administration. The at least one efficacy measurement is chosen from PANSS total score, PANSS positive, PANSS negative, GPP subscale, BPRS, and responder rates.

Another embodiment of the present disclosure is directed to a method for treating schizophrenia in a patient in need thereof comprising: administering to the patient a composition comprising a dose of at least one bifeprunox compound ranging from about 20 mg to about 30 mg over a treatment period, wherein the composition is effective to achieve a reduction in at least one symptom or an improvement in at least one symptom of schizophrenia in the patient, from a baseline measurement before administration.

An embodiment of the present invention relates to at least one compound of bifeprunox for use in the treatment of patients (such as, schizophrenics) with psychoses and mood disorders wherein bifeprunox (i.e. at least one bifeprunox compound) is administered in combination with the mood-stabilizing drug lithium, and a kit for said use.

A further embodiment of the present invention relates to bifeprunox for use in the treatment of patients with at least one condition chosen from schizophrenia and depression wherein at least one bifeprunox compound is administered in combination with an antidepressant (for example, an SSRI, such as paroxetine), and a kit for the same.

Further embodiments of the present disclosure relate to methods for co-administration of bifeprunox with a CYP2C9 inhibitor (for example, fluconazole), with a CYP3A4 inhibitor (for example, ketoconazole and carbamazepine), with a CYP2D6 inhibitor (for example, paroxetine) and with a H2-antagonist (for example, famotidine), respectively, and kits for the use thereof.

In another embodiment of the present disclosure, the at least one bifeprunox compound comprises bifeprunox mesylate. In addition, the at least one bifeprunox compound is bifeprunox mesylate. The bifeprunox mesylate may be chosen from the α, γ, or δ crystalline polymorphic forms, and mixtures thereof. For example, the at least one bifeprunox compound comprises at least one polymorphic form chosen from the α and γ polymorphic forms.

A further embodiment of the present disclosure relates to a method for treating schizophrenia in a patient in need thereof comprising: administering to the patient a composition comprising: a dose of at least one bifeprunox compound, the dose ranging from about 20 mg to about 30 mg, wherein, over a treatment period, the composition is effective to achieve: (1) treatment of at least one symptom of schizophrenia; and (2) either: (a) an absence of at least one side-effect associated with schizophrenia therapy with a first generation atypical antipsychotic; or (b) a reduced presence of at least one side-effect associated with schizophrenia therapy with a first generation atypical antipsychotic; in a patient administered the composition. The at least one side-effect associated with schizophrenia therapy with a first generation atypical antipsychotic is chosen from weight gain, disorders of triglyceride levels and total cholesterol, hyperglycemia, diabetes-related adverse events, dyslipidemia, and combinations thereof.

In yet a further embodiment, the present disclosure is directed to a method for treating schizophrenia in a patient in need thereof comprising: administering to the patient a composition comprising a dose of at least one bifeprunox compound ranging from about 20 mg to about 30 mg over a treatment period, wherein the administered composition results in at least two measurements associated with a favorable metabolic profile chosen from no weight gain, reduction in weight gain, no increase in prolactin, reduction in triglyceride level, reduction in cholesterol, no glucose dysregulation, and no QTc prolongation, compared with baseline measurements before administration.

A still further embodiment of the present disclosure is directed to a method for treating schizophrenia in a patient in need thereof comprising: administering to the patient a composition comprising a therapeutically effective dose of at least one bifeprunox compound, wherein administration over a treatment period results in a maintenance or a reduction in weight, or avoids an increase in weight of the patient, in comparison to a baseline measurement before administration.

As used herein, the term “reduction,” in addition to including reducing or lowering, also encompasses regression, lack of expression or avoidance of, e.g., a symptom, side effect, condition, disease or disorder. Further, the term “first-generation atypical antipsychotic(s)” refers to a compound(s) that act as dopamine D2 receptor antagonists and, most of them, also as serotonin receptor antagonists, such as, but not limited to, aripirazole, amisulpride, clozapine, olanzapine, quetiapine, risperidone, ziprasidone, and zotepine. First-generation atypical antipsychotics differ from bifeprunox compounds in that bifeprunox compounds act as highly potent partial D2 agonists and also as moderately potent serotonin 5-HT1 A agonists, and, in addition, bifeprunox compounds further, in contrast to many antipsychotic compounds, show no appreciable affinity for 5-HT2A and 5-HT2C, muscarinic and histaminergic receptors.

As used herein, the term “treatment” or “treating” refers to any treatment of a mammalian, for example, a human condition or disease, and includes: (1) inhibiting the progression or development of the disease or condition, (2) relieving the disease or condition, i.e., causing the disease or condition to regress, or (3) stopping one or more symptoms of the disease or condition.

As used herein, the term “symptom” refers to any sensation or change in bodily function that is experienced by a patient and is associated with a particular disease. Further, the term “efficacy measurement” can at least include, but is not limited to, the Positive and Negative Symptom Scale (PANSS) total score, the positive symptom sub-scale score of the PANSS, the negative symptom sub-scale score of the PANSS, the general psychopathology sub-scale of the PANSS, the Brief Psychiatric Rating Scale (BPRS) total score derived from the PANSS, the BPRS psychosis derived from the PANSS, the Clinical Global Impressions Severity of Illness score (CGI-S), the Clinical Global Impressions Improvement score (CGI-I), and responder rate based on the PANSS total score and CGI-I responder rate, weight, vital signs (including pulse rate and systolic/diastolic blood pressure (BP)—both lying after five minutes and standing after two minutes, and oral temperature), 12-lead electrocardiogram (ECG), safety laboratory assessments including hematology, biochemistry, urinalysis, adverse event monitoring, and assessments of abnormal movement including the Simpson-Angus Scale (SAS), Barnes Akathisia Scale (BAS), Abnormal Involuntary Movement Scale (AIMS), and combinations thereof.

As used herein, the term “therapeutically effective” refers to an amount of a therapeutic agent capable of treating a condition treatable by administrating a composition of the invention. That amount is the amount sufficient to achieve a detectable therapeutic or ameliorative response in a tissue system, animal or human. The effect may include, for example, treating the conditions listed herein. The precise effective amount for a subject will depend upon the subject's size and health, the nature and extent of the disease or condition being treated, recommendations of the treating physician (researcher, veterinarian, medical doctor or other clinician), and the therapeutics, or combination of therapeutics, selected for administration.

The term “treatment period” can encompass a duration of therapy including, but not limited to, 1 month, 6 weeks, 2 months, 3 months, 4 months, 5 months, 6 months, or any duration in between, and includes any combination of time segments, during which the patient remains under therapy.

The crystalline polymorphic form of a bifeprunox mesylate according to the present disclosure is defined by at least the physicochemical parameters as disclosed in International Publication No. WO 05/016898.

In another embodiment, the present disclosure provides bifeprunox mesylate in which at least about 50 percent by weight (wt. %), at least about 60 wt. %, at least about 70 wt. %, at least about 80 wt. %, at least about 90 wt. %, or at least about 95 wt. % of the bifeprunox mesylate is in the polymorphic α form. In another embodiment, the pharmaceutical composition is substantially devoid of any γ or δ polymorphic forms of bifeprunox mesylate. In another embodiment, the bifeprunox mesylate provided by the present disclosure comprises less than 10 wt. %, less than 5 wt. %, or less than 2.5 wt. % of the γ or δ polymorphic forms of bifeprunox mesylate. In another embodiment, at least about 99 wt. % of bifeprunox mesylate is in the polymorphic α form.

The preparation of polymorphic form α can be carried out according to the procedures described in International Publication No. WO 05/016898.

The at least one bifeprunox compound according to the present disclosure can be formulated into dosage forms in which the active substance is present in the solid, liquid, or powder form by methods known in the art. Examples of said dosage forms are (optionally coated) tablets, capsules, granular aerosols, suppositories and suspensions. Such dosage forms can be prepared by mixing the at least one bifeprunox compound with inert pharmaceutically acceptable excipients and/or carriers to prepare a pharmaceutical composition.

Pharmaceutical compositions of the present disclosure can comprise at least one pharmaceutical excipient. Non-limiting examples of suitable excipients include suspending agents (for example, gums, xanthans, cellulosics and sugars), humectants (for example, sorbitol), solubilizers (for example, ethanol, water, PEG and propylene glycol), surfactants (for example, sodium lauryl sulfate, Spans, Tweens, and cetyl pyridine), preservatives, antioxidants (for example, parabens, and vitamins E and C), anti-caking agents, coating agents, chelating agents (for example, EDTA), stabilizers, antimicrobial agents, antifungal or antibacterial agents (for example, parabens, chlorobutanol, phenol, sorbic acid), isotonic agents (for example, sugar, sodium chloride), thickening agents (for example, methyl cellulose), flavoring agents (for example, chocolate, thalmantin, aspartame, root beer or watermelon or other flavorings stable at pH 7 to 9), anti-foaming agents (e.g., simethicone, Mylicon®), disintegrants, flow aids, lubricants, adjuvants, colorants, diluents, moistening agents, preservatives, carriers, binders (for example, hydroxypropylmethylcellulose, polyvinyl pyrilodone, other cellulosic materials and starch), diluents (for example, lactose and other sugars, starch, dicalcium phosphate and cellulosic materials), disintegrating agents (for example, starch polymers and cellulosic materials), glidants and water insoluble or water soluble lubricants or lubricating agents.

One illustrative dosage form comprises, apart from the milled and sieved dose of the active substance (bifeprunox as described herein), lactose monohydrate, microcrystalline cellulose, sodium starch glycolate (for example, type A), sodium stearyl fumarate and optionally colloidal anhydrous silica. In one embodiment, lactose is present in an amount ranging from about 20% to about 90% by weight, from about 70% to about 90% by weight, or from about 75% to about 85% by weight, based on the total weight of the tablet core. Microcrystalline cellulose is present in an amount ranging from about 5% to about 90% by weight, from about 10% to about 15% by weight, or from about 11% to about 12% by weight, based on the total weight of the tablet core. Sodium starch glycolate (e.g. type A) is present in an amount ranging from about 0.1% to about 2.5% by weight, from about 0.3% to about 0.7% by weight, or from about 0.5% by weight, based on the total weight of the tablet core. Sodium stearyl fumarate is present in an amount ranging from about 0.1% to about 1.5% by weight, from about 0.6% to about 1.3% by weight, or from about 1.0% by weight, based on the total weight of the tablet core. Colloidal anhydrous silica is optionally added to the formulation in order to improve the flow properties of the powder. If desired, colloidal anhydrous silica is typically present in an amount ranging from about 0.05% to about 0.5% by weight or from about 0.4% by weight, based on the total weight of the tablet core. The amount of optional coating ranges from about 2.0% to about 5.0% by weight, from about 3.0% to about 4.0% by weight, or from about 3.5% by weight, based on the total weight of the tablet core.

In at least one embodiment, the pharmaceutical compositions comprising the at least one bifeprunox compound according to the present disclosure can be administered to a subject, for example a human subject, in need thereof.

The present disclosure is also directed to, but not limited to, reducing a PANSS total score in a patient, maintaining or reducing body weight, maintaining and/or improving triglycerides levels and/or total cholesterol levels, maintaining clinical stability of schizophrenia, improving one or more psychotic symptoms or maintaining an EPS profile similar to baseline measurements before administration. The present disclosure is also directed to methods for avoiding or reducing the incidence of hyperglycemia and/or diabetes-related adverse events. These methods are exemplified in the following clinical examples provided below.

### EXAMPLES

The following examples are only intended to further illustrate the present disclosure, in more detail, and therefore these examples are not deemed to restrict the scope of the present disclosure in any way.

### Example 1

### Efficacy of Bifeprunox in the Treatment of Schizophrenia

A six-week, randomized, double-blind, placebo-controlled and risperidone referenced study was used to assess the efficacy and safety of fixed doses of bifeprunox in the treatment of schizophrenia. A total of 599 subjects were randomized.

Treatment started with a single-blind placebo lead-in period of at least three days, followed by titration from 0.25 mg up to 30 mg/day or 40 mg/day of bifeprunox for bifeprunox-treated subjects. Risperidone-treated subjects were titrated from 2 mg to 6 mg daily over a three-day period and maintained at 6 mg/day for the remainder of the treatment period.

Rating scale assessments were performed weekly, except for week 5, to measure the change from baseline to endpoint of the PANSS total score. Other assessments included: PANSS positive symptom subscale score, PANSS negative symptom subscale score, PANSS general pschopathology subscale score, BPRS total score, BPRS psychosis score, the CGI-S score, the CGI-I score, responder rates based on the PANSS total score, and the Calgary Depression Scale for Schizophrenia (CDSS).

Safety and tolerability measures included physical examinations, weight, waist circumference, vital signs, 12-lead electrocardiogram (ECG), clinical laboratory assessments (hematology, biochemistry, urinalysis, special laboratory assessments for prolactin, IGF-1, IGFBP-3, thyroid function), need for anticholinergic treatment during double-blind treatment period, concomitant medication use, adverse event monitoring and assessments of normal movement.

The 20 mg bifeprunox treatment group showed a statistically significant difference from placebo for the primary endpoint of change from baseline to endpoint in PANSS total score. The mean change (standard deviation) from baseline to endpoint in PANSS total score was −13.5 (20.1) for the 30 mg bifeprunox group, −10.3 (20.5) for the 40 mg bifeprunox group, −7.7 (19.2) for the placebo group, and −19.7 (19.3) for the risperidone group.

The 30 mg bifeprunox group showed notable differences from placebo for CGI-S score, PANSS negative symptom subscale score, and PANSS positive symptom subscale score. Notable differences were also observed between the 30 mg bifeprunox group and placebo for the change from baseline to endpoint in PANSS general psychopathology subscale score, BPRS total score, BPRS psychosis cluster score, PANSS responder rate, and CGI-I responder rate. In the study, a PANSS responder refers to a subject whose PANSS total score decreased by 20% or more from baseline to endpoint. A CGI-I responder refers to a subject who was categorized as “very much improved” or “much improved” in the CGI Global Improvement scale at endpoint.

The 40 mg bifeprunox group showed a notable difference from placebo for the change in PANSS positive symptom subscale score and BPRS psychosis cluster score.

Decreases in body weight were seen in bifeprunox treatment groups in contrast to an increase in the placebo and risperidone treatment groups.

The bifeprunox groups had a lower incidence of N to H shifts in triglycerides, VLDL, and LDL, and a higher incidence of N to L shifts in total cholesterol and VLDL compared with the placebo and risperidone groups.

### Example 2

### Clinical Studies Directed to Bifeprunox in the Treatment of Schizophrenia

### Example 2a

### Clinical Study One

OBJECTIVES: This clinical study's primary objective was to investigate whether six weeks of treatment with 5 mg, 10 mg, or 20 mg bifeprunox is superior to treatment with placebo in adult subjects with schizophrenia, using the change from Baseline to Endpoint of the Positive and Negative Symptom Scale (PANSS) total score as the primary outcome measure. The secondary objectives were to assess the efficacy of bifeprunox in treating schizophrenia using the positive symptom sub-scale score of the PANSS, the negative symptom sub-scale score of the PANSS, the general psychopathology sub-scale of the PANSS, the Brief Psychiatric Rating Scale (BPRS) total score derived from the PANSS, the BPRS psychosis derived from the PANSS, the Clinical Global Impressions Severity of Illness score (CGI-S), the Clinical Global Impressions Improvement score (CGI-I), and responder rate based on the PANSS total score and CGI-I responder rate. It was also an objective of this study to assess the safety and tolerability of bifeprunox using physical examination, weight, vital signs (including pulse rate and systolic/diastolic blood pressure (BP)—both lying after five minutes and standing after two minutes, and oral temperature), 12-lead electrocardiogram (ECG), safety laboratory assessments including hematology, biochemistry, and urinalysis, need for anticholinergic treatment during double-blind treatment period, concomitant medication use, adverse event monitoring, and assessments of abnormal movement including the Simpson-Angus Scale (SAS), Barnes Akathisia Scale (BAS), and Abnormal Involuntary Movement Scale (AIMS).

METHODOLOGY: This was a randomized, double-blind, fixed-dose, placebo-controlled, risperidone-referenced, parallel group, multi-center study in adult subjects with schizophrenia. There were five treatment groups in this study. The treatment groups were as follows: bifeprunox 5 mg, bifeprunox 10 mg, bifeprunox 20 mg, risperidone 6 mg and placebo. Study medication was administered once daily. After Baseline measurements were performed, the titration phase was begun. Bifeprunox treated subjects were titrated up to 5 mg, 10 mg or 20 mg according to a standardized titration schedule (Day 1: 0.125 mg, Day 2: 0.25 mg, Day 3: 0.5 mg, Day 4: 1.0 mg, Day 5: 2.0 mg, Day 6: 5.0 mg, Day 7: 10.0 mg, Day 8: 20 mg). When the assigned dose was reached, subjects were maintained at that dose for the remainder of the six-week treatment period. Risperidone treated subjects were titrated to 6 mg over three days (Day 1: 2 mg, Day 2: 4 mg, Day 3: 6 mg) using a once-daily regimen.

Number of Subjects (Planned, Screened, Randomized and Analyzed): A total of 575 patients with Schizophrenia were planned for inclusion in the study. A total of 836 subjects were screened at 40 centers and a total of 589 subjects (5 mg bifeprunox: 115 subjects; 10 mg bifeprunox: 120 subjects; 20 mg bifeprunox: 115 subjects; placebo: 119 subjects; risperidone: 120 subjects) were randomized at 37 centers.

Test Product. Dose and Mode of Administration: Bifeprunox tablets, total daily dose 0.125 mg to 20 mg administered orally using a once daily dosing regimen.

Reference There, Dose and Mode of Administration: Placebo and risperidone, 2 mg to 6 mg administered orally using a once daily dosing regimen.

Efficacy Results: The 20 mg bifeprunox dose was effective in reducing both positive and negative symptoms of schizophrenia and lessening overall psychopathology compared to placebo as shown by the statistically significant comparisons from the analysis of PANSS total and subscale scores. Subjects in the 20 mg bifeprunox group had a 5.8 point greater improvement from Baseline in PANSS total score compared with placebo subjects. The lower doses of bifeprunox were not effective. The 10 mg dose of bifeprunox did not show statistically greater improvement for any efficacy endpoint. The 5 mg dose showed greater improvement on some secondary efficacy measures compared to placebo, but did not show superiority to placebo for the primary efficacy endpoint (PANSS total score), Risperidone 6 mg was used as an active reference in this study and showed clear separation from the placebo group. In general, the magnitude of the improvements seen in the 20 mg bifeprunox group was smaller than those seen in the risperidone group for most efficacy endpoints.

Safety Results: The percentage of subjects with at least one treatment-emergent adverse event (TEAE) was similar across treatment groups: 89% (102 subjects) in the 5 mg bifeprunox group, 87% (104 subjects) in the 10 mg bifeprunox group, 83% (95 subjects) in the 20 mg bifeprunox group, 85% (101 subjects) in the placebo group, and 89% (107 subjects) in the risperidone group. Overall, out of 349 subjects treated with bifeprunox, the most frequently reported TEAEs were headache, dyspepsia, insomnia, nausea, vomiting NOS, constipation, and agitation. TEAEs with a higher incidence (≧5% difference) in the 20 mg bifeprunox group (N=114 subjects) compared with the placebo group (N=119 subjects) included constipation, dyspepsia, and vomiting NOS. Related TEAEs with a higher incidence (≧5% difference) in the 20 mg bifeprunox group compared with placebo were constipation, vomiting NOS, and headache NOS. The percentage of subjects with at least one severe TEAE was lowest in the 20 mg bifeprunox group (11 subjects, 10%) followed by the placebo group (15 subjects, 13%). For the remaining groups, the percentage of subjects with at least one severe TEAE ranged from 16% to 18%. The incidence of TEAEs considered to be severe was similar (<5% difference) between the 20 mg bifeprunox group and the placebo group for all TEAEs. There were no dose-related trends in the bifeprunox groups in overall incidence, incidence of related, or incidence of severe TEAEs observed for any event.

The total number of subjects with at least one SAE was higher in the active treatment groups (bifeprunox groups: 12-15%, risperidone group: 16%) compared with the placebo group (9%). The most commonly reported SAEs (≧5% in any treatment group) were aggravated psychosis and aggravated schizophrenia NOS. There was a slightly higher incidence of aggravated schizophrenia NOS in the 20 mg bifeprunox group (8 subjects, 7%) compared with placebo and risperidone (5 subjects each, 4%). Suicide attempt was reported for one subject in the 10 mg bifeprunox group (<1%), two subjects in the 20 mg bifeprunox group (2%), no subjects in the placebo group, and no subjects in the risperidone group. The serious adverse event of suicidal ideation was reported for one subject each (<1%) in the 10 mg bifeprunox and risperidone groups (one additional subject in the 20 mg bifeprunox group experienced a non-serious adverse event of suicidal ideation). No trend of dose-related increase in incidence of any SAE was observed for the bifeprunox groups. Bifeprunox was safe and well tolerated at all dose levels. The total number of subjects with at least one AE that led to discontinuation was similar among treatment groups (5 mg bifeprunox: 13 subjects, 11%; 10 mg bifeprunox: 17 subjects, 14%; 20 mg bifeprunox: 11 subjects, 10%; placebo: 15 subjects, 13%; risperidone: 17 subjects, 14%). The most common (reported by >2% of subjects in any treatment group) AEs that led to discontinuation were agitation, aggravated psychosis, and aggravated schizophrenia NOS. There were no treatment group differences in incidence of AEs leading to discontinuation of study medication between the 20 mg bifeprunox group and the placebo group. There was no trend of dose-related increase in incidence of AEs leading to discontinuation in the bifeprunox groups. Evaluation of laboratory, vital sign, ECG and physical exam findings did not raise any unexpected safety concerns. Bifeprunox subjects exhibited a decrease in prolactin compared with the placebo group. Mild weight loss was observed in the bifeprunox groups but not in the placebo or risperidone groups.

There were no notable differences between treatment groups in changes from Baseline to Endpoint in BAS, SAS, or AIMS scores. Use of anticholinergic medication for patients treated with bifeprunox was similar to that of patients on placebo and less than in patients on risperidone.

CONCLUSION: One conclusion of this study is that a 20 mg dose of bifeprunox given once daily over a treatment period of six weeks was effective in reducing both positive and negative symptoms of schizophrenia. Overall, all doses of bifeprunox were safe and well tolerated by schizophrenic subjects. No dose-response relationship of safety/tolerability was seen.

### Example 2b

### Clinical Study Two

PRIMARY OBJECTIVES: To investigate whether six weeks of treatment with fixed doses of bifeprunox (30 mg/day or 40 mg/day) can demonstrate superior efficacy compared with placebo in adult subjects with schizophrenia, using the change from Baseline to Endpoint of the Positive and Negative Symptom Scale (PANSS) total score as the primary outcome.

SECONDARY OBJECTIVES: To assess the efficacy of bifeprunox in treating schizophrenia using the positive symptom sub-scale score of the PANSS, the negative symptom sub-scale score of the PANSS, the general psychopathology sub-scale of the PANSS, Brief Psychiatric Rating Scale (BPRS) total score derived from the PANSS, BPRS psychosis score derived from the PANSS, the Clinical Global Impression Severity of Illness score (CGI-S), the Clinical Global Impression Improvement score (CGI-I), responder rate based on the PANSS total score, Calgary Depression Scale for Schizophrenia (CDSS), and subject satisfaction rating. Pharmacokinetic (PK) data of bifeprunox in schizophrenic subjects were also assessed and are presented in a separate report (combined with data from study S1543003). To assess the safety and tolerability of bifeprunox using physical examination, weight, waist circumference, vital signs (pulse rate and systolic/diastolic blood pressure [BP]-both lying after five minutes and standing after two minutes, and oral temperature), 12-lead electrocardiogram (ECG), clinical laboratory assessments (hematology, biochemistry, urinalysis, special laboratory assessments for prolactin, IGF-1, IGFBP-3, and thyroid function), need for anticholinergic treatment during double-blind treatment period, concomitant medication use, adverse event (AE) monitoring, and assessments of abnormal movement including the Simpson-Angus Scale (SAS), Barnes Akathisia Scale (BAS), and Abnormal Involuntary Movement Scale (AIMS).

METHODOLOGY: This was a Phase III, six-week randomized, double-blind, placebo-controlled, risperidone-referenced, parallel-group, multi-center study of the efficacy, tolerability, and safety of bifeprunox in adult subjects with schizophrenia. Subjects who completed this study had the option to continue in the long-term. The treatment groups were: bifeprunox 30 mg/day, bifeprunox 40 mg/day, risperidone 6 mg/day, and placebo. After completing a Single-Blind placebo lead-in period of at least three days, bifeprunox-treated subjects were titrated from 0.25 mg up to 30 mg/day or 40 mg/day according to a standardized titration scheme over an eight-day period. When the assigned dose was reached, subjects were maintained at that dose for the remainder of the six week treatment period. Risperidone-treated subjects were titrated from 2 mg to 6 mg daily over a three-day period and were subsequently maintained at 6 mg/day for the remainder of the treatment period. Rating scale assessments for efficacy and abnormal movement disorders were done weekly except for Week 5. Safety assessments were performed at Screening, during treatment and at the end of the study. Subject satisfaction with study medication was assessed at Week 6. Samples of blood were obtained at Weeks 2, 4, and 6 for determination of bifeprunox in plasma. Blood samples were also obtained at Screening/Baseline, Week 3, and Week 6 for clinical laboratory assessments.

Number of Subjects (Planned, Consented, Randomized and Analyzed): A total of 576 subjects with schizophrenia were planned for inclusion in the study. Of 783 screened subjects, a total of 599 subjects were randomized (30 mg bifeprunox: 148 subjects, 40 mg bifeprunox: 148 subjects; placebo: 149 subjects; risperidone: 154 subjects).

Diagnosis and Main Criteria for Inclusion: Male or female subjects 18-75 years of age with schizophrenia (per DSM-IV-TR criteria). Subjects had to have a total score on the PANSS between 70 and 120; at least two of four PANSS items (conceptual disorganization, hallucinatory behavior, suspiciousness, unusual thought content) had to have a score ≧4; and the score on the CGI-S had to be at least 4.

Reference Therapy, Dose and Mode of Administration: Placebo and risperidone, 6 mg administered orally once daily.

Efficacy Results: The 30 mg bifeprunox treatment group showed a statistically significant difference from placebo for the primary endpoint of change from Baseline to Endpoint in PANSS Total Score (LOCF) based on the Hochberg adjusted p-value (adjusted p=0.020). The 40 mg bifeprunox treatment group was not significantly different from the placebo group (adjusted p=0.156) for the primary efficacy endpoint. The mean change (SD) from Baseline to Endpoint in PANSS total score was −13.5 (20.1) for the 30 mg bifeprunox group, −10.3 (20.5) for the 40 mg bifeprunox group, −7.7 (19.2) for the placebo group, and −19.7 (19.3) for the risperidone group. The treatment effect values (for mean change from Baseline at Endpoint [LOCF]) corresponding to the difference between bifeprunox and placebo were: −5.9 for the 30 mg bifeprunox group and −3.2 for the 40 my bifeprunox group. No statistically significant difference based on the planned step-down procedure was seen in change from Baseline to Endpoint in CGI-S for the 30 mg bifeprunox treatment group compared to placebo. Consequently, the differences between placebo and 30 mg bifeprunox groups for change from Baseline to Endpoint in PANSS Negative Symptom and PANSS Positive Symptom subscale scores were not evaluated using the stepdown procedure for statistical significance compared to placebo. The 30 mg bifeprunox group showed notable differences from placebo for CGI-S score (nominal p=0.028), PANSS Negative Symptom subscale score (nominal p=0.027), and PANSS Positive Symptom subscale scores (nominal p=0.010).

Notable differences were observed between the 30 mg bifeprunox group and placebo for the change from Baseline to Endpoint in PANSS general psychopathology subscale score (p=0.025), BPRS total score (p=0.019), BPRS psychosis cluster score (p=0.002), PANSS (30%) responder rate (p=0.019), and CGI-I responder rate (p=0.039). No notable differences at Endpoint were observed between the 30 mg bifeprunox group and placebo for subject satisfaction (p=0.051), CGI Improvement score, or CDSS score. For the 40 mg bifeprunox group, no statistically significant difference from placebo was seen for the primary efficacy parameter. No notable differences from placebo were noted for the 40 mg bifeprunox group for any of the secondary efficacy parameters with the exception of PANSS Positive Symptom Subscale score (p=0.020) and BPRS Psychosis cluster score (p=0.031).

Safety Results: The percentage of subjects with at least one TEAE was comparable in the bifeprunox dose groups (74%-76%) and was higher than the placebo group (64%) but slightly lower than in the risperidone group (78%). Treatment-emergent AEs with a higher (≧5% difference) incidence in the bifeprunox groups compared with the placebo group included nausea, vomiting, constipation, dyspepsia, diarrhoea, and dizziness. No difference between bifeprunox treatment groups was generally observed in the incidence of individual TEAEs in the bifeprunox groups. Of TEAEs occurring in at least 5% of subjects in any treatment group, those having a higher (≧2% difference) incidence in the 40 mg bifeprunox group compared with the 30 mg bifeprunox group included nausea, vomiting, toothache, anorexia, akathisia, dizziness, headache, and insomnia. In contrast, dry mouth, salivary hypersecretion, decreased appetite, sedation, somnolence, anxiety, and vaginitis occurred with a higher (≧2% difference) incidence in the 30 mg bifeprunox group compared with the 40 mg bifeprunox group. The incidence of severe TEAEs was generally low (≦1% incidence) with the exception of TEAEs of psychotic disorder (≦6%) and schizophrenia (3% each in the bifeprunox and placebo groups and 2% in the risperidone group). The incidence of severe TEAEs was generally comparable across treatment groups and there was no difference between bifeprunox treatment groups in the incidence of severe TEAEs for any event. Special interest TEAEs were defined prior to database lock and included events related to suicide, suicide attempt, sexual dysfunction, syncope, vasovagal attack, and postural hypotension. A total of 76 subjects (13%) overall reported at least one TEAE of special interest during the study. The percentage of subjects with at least one special interest TEAE was higher in the bifeprunox and risperidone treatment groups (12%-16%) compared with the placebo group (6%). The majority of TEAEs of special interest occurred in ≦1% of subjects in any treatment group. Dizziness was the most commonly reported and occurred at a slightly higher incidence in the bifeprunox treatment groups compared with the other two groups.

A total of 60 subjects (10%) overall experienced 81 SAEs (including deaths). Most SAEs occurred in ≦1% of subjects. The exceptions were psychotic disorder (≦5%) and schizophrenia (3% in each of the four treatment groups). The incidence of SAEs was comparable among bifeprunox groups and placebo. There were no notable differences between treatment groups on measures of abnormal movement (BAS, SAS, or AIMS scores). Use of anticholinergic medication for subjects treated with bifeprunox was similar to that of subjects treated with placebo. Overall, there were no clinically meaningful changes in clinical laboratory parameters, physical examination findings, or ECG readings with bifeprunox. The bifeprunox groups had a lower incidence of N to H shifts in triglycerides, VLDL, and LDL and a higher incidence of N to L shifts in total cholesterol and VLDL compared with the placebo and risperidone groups. The incidence of markedly abnormal total cholesterol values was similar across treatment groups (1% to 2%). Markedly abnormal triglyceride values were reported by slightly fewer subjects in the 40 mg bifeprunox group compared with other groups. There was a slightly greater incidence of N to H shifts in tri-iodine thyronine in the 30 mg bifeprunox and risperidone groups (6% each) compared with the placebo (4%) and 40 mg bifeprunox (5%) groups and slightly greater incidence of N to L shifts in TSH in the 30 mg bifeprunox (4%) and the 40 mg bifeprunox (5%) compared with the placebo (2%) and risperidone (<1%) groups. Small comparable decreases in body weight were seen in bifeprunox treatment groups in contrast to an increase in the placebo and risperidone treatment groups. The incidence of markedly abnormal decreases in body weight was slightly higher in both the bifeprunox treatment groups relative to the placebo and risperidone groups, while the incidence of markedly abnormal increases in body weight was higher in the risperidone treatment group relative to the bifeprunox and placebo groups.

CONCLUSION: One conclusion of this study is that a 30 mg dose of bifeprunox given once daily over a treatment period of six weeks was effective in reducing the symptoms of schizophrenia as shown by the statistically significant comparisons from the analysis of PANSS total scores. Subjects in the 30 mg bifeprunox group had a 5.9 point greater improvement from Baseline to Endpoint in PANSS total score compared with placebo subjects based on LOCF data. The statistically significant differences noted between the active control, risperidone and placebo treatments for the primary and secondary efficacy parameters demonstrate that this is a valid study.

The 30 mg bifeprunox treatment group showed a notable difference from placebo (based on nominal p-values) for the three key secondary efficacy endpoints, change from Baseline to Endpoint in CGI-S, PANSS Negative, and Positive Symptom subscale scores; statistical significance was not achieved based on the step-down procedure for these three key secondary efficacy endpoints. Notable differences were observed between the 30 mg bifeprunox group and placebo at Endpoint for most of the other secondary efficacy parameters (change from Baseline to Endpoint in PANSS general psychopathology subscale score, BPRS total score, and BPRS psychosis cluster score). Notable differences between the 30 mg bifeprunox dose and placebo were also demonstrated at Endpoint for PANSS and CGI-I Responder rates. The 40 mg bifeprunox treatment group did not show statistically significant differences from placebo for the primary efficacy parameter. There were no notable differences between the 40 mg bifeprunox and placebo groups for any of the secondary efficacy parameters with the exception of PANSS Positive Symptom Subscale and BPRS cluster score. Overall, 30 mg and 40 mg doses of bifeprunox were safe and well tolerated by schizophrenic subjects.

### Example 2c

### Clinical Study Three

PRIMARY OBJECTIVE: To investigate whether six weeks of treatment with fixed doses of bifeprunox (20 mg/day or 30 mg/day) can demonstrate superior efficacy compared with placebo in adult subjects with schizophrenia, using the change from Baseline to Endpoint of the Positive and Negative Symptom Scale (PANSS) total score as the primary outcome.

SECONDARY OBJECTIVES To assess the efficacy of bifeprunox in treating schizophrenia using the Positive Symptom sub-scale score of the PANSS, the Negative Symptom sub-scale score of the PANSS, the General Psychopathology sub-scale of the PANSS, Brief Psychiatric Rating Scale (BPRS) total score derived from the PANSS, BPRS psychosis score derived from the PANSS, the Clinical Global Impression Severity of Illness score (CGI-S), the Clinical Global Impression Improvement score (CGI-I), CGI-I responder rate, PANSS responder rate based on the PANSS total score, Calgary Depression Scale for Schizophrenia (CDSS), and Subject Satisfaction rating. To assess the safety and tolerability of bifeprunox using physical examination, weight, waist circumference, vital signs (pulse rate and systolic/diastolic blood pressure [BP], and oral temperature), 12-lead electrocardiogram (ECG), clinical laboratory assessments (hematology, biochemistry including fasting insulin level, fasting glucose, fasting lipid profile, urinalysis, special laboratory assessments for prolactin, IGF-1, IGFBP-3, and thyroid function), need for anticholinergic treatment during double-blind treatment period, concomitant medication use, adverse event (AE) monitoring, and assessments of abnormal movement including the Simpson-Angus Scale (SAS), Barnes Akathisia Scale (BAS), and Abnormal Involuntary Movement Scale (AIMS).

METHODOLOGY: This was a Phase III, six week randomized, double-blind, placebo-controlled, olanzapine-referenced, parallel group, multi-center study of the efficacy, tolerability, and safety of bifeprunox in adult subjects with schizophrenia. There were four treatment arms in this study, approximately 144 subjects per arm. The treatment groups were: bifeprunox 20 mg/day, bifeprunox 30 mg/day, olanzapine 15 mg/day, and placebo. After completing the Single-Blind Placebo Lead-in Period of at least three days, bifeprunox-treated subjects were titrated from 0.25 mg up to 20 mg/day or 30 mg/day according to a standardized titration scheme over a seven- or eight-day period, respectively. When the assigned dose was reached, subjects were maintained at that dose for the remainder of the six week treatment period. Olanzapine-treated subjects began dosing at 10 mg/day for the initial seven day period and, were subsequently maintained at 15 mg/day for the remainder of the treatment period. Subjects were hospitalized (if not already inpatients) after eligibility for study entry was verified, starting from the Screening Visit until at least 10 days after Baseline. Subjects could be hospitalized longer than 10 days if considered medically necessary by the Investigator. Rating scale assessments for efficacy and abnormal movement disorders were done weekly except for Week 5. Safety assessments were performed at Screening, during treatment and at the end of the study. Subject satisfaction with study medication was assessed at Week 6. Samples of whole blood were obtained at Weeks 2, 4, and 6 for determination of bifeprunox in plasma.

Number of Subjects (Planned, Consented, Randomized and Analyzed): A total of 576 subjects with schizophrenia were planned for inclusion in the study. A total of 814 subjects were screened at 32 centers and a total of 604 subjects (20 mg bifeprunox; 154 subjects, 30 mg bifeprunox: 150 subjects; placebo: 150 subjects; olanzapine: 150 subjects) were randomized at 32 centers.

Diagnosis and Main Criteria for Inclusion: Male or female subjects 18-75 years of age with schizophrenia (per DSM-IV-TR criteria), Subjects must have had a total score on the PANSS between 70 and 120; at least two of four PANSS items (conceptual disorganization, hallucinatory behavior, suspiciousness, unusual thought content) must have had a score ≧4; the score on the CGI-S must have been at least 4.

Test Product, Dose and Mode of Administration: Bifeprunox tablets, total daily dose 20 mg or 30 mg (one 20 mg tablet and one 10 mg tablet) administered orally using a once daily dosing regimen.

Reference Therapy, Dose and Mode of Administration: Placebo and olanzapine, 5 mg and 15 mg administered orally using a once daily dosing regimen.

Efficacy Results: Both the 20 mg and 30 mg treatment groups showed improvements over Baseline at Endpoint but did not demonstrate efficacy as compared to the placebo group with respect to the primary and key secondary efficacy parameters. However, the mg bifeprunox group showed notable improvement over the placebo group for the change from Baseline to Endpoint in a secondary efficacy parameter, CGI improvement score (nominal p=0.027). Additionally in one other secondary efficacy parameter, the difference between the mg bifeprunox group and the placebo group approached being notable (p=0.061) for PANSS-20%-responder rate. However, these occurrences of notable and nearly notable differences among the secondary efficacy parameters do not exceed what is expected to happen by chance, i.e., in 5% of the treatment comparisons. In all other secondary and key secondary parameters, neither bifeprunox treatment group showed notable improvement over the placebo group for any efficacy endpoint. Olanzapine at a dose of 15 mg was used as an active reference in this study. The difference of the PANSS total score between olanzapine and placebo was analyzed in accordance with sensitivity analyses. These results showed that olanzapine was notably different from placebo (p<0.001). In general, the magnitude of the improvements seen in the bifeprunox dose groups was higher than those seen in the placebo group, but lower than those seen in the olanzapine group for most efficacy endpoints.

Safety Results: The percentage of subjects with at least one treatment-emergent adverse event (TEAE) was highest in the 20 mg bifeprunox group (126 subjects, 82%) followed by the 30 mg bifeprunox (115 subjects, 77%), olanzapine (110 subjects, 73%), and placebo (107 subjects, 72%) groups. Overall, out of 304 subjects treated with bifeprunox, the most frequently reported TEAEs were headache, nausea, vomiting, dyspepsia, and insomnia. TEAEs with a higher incidence (≧5% difference) in the bifeprunox groups compared with the placebo group included nausea, vomiting, and constipation. In general, no clear dose-related increase in the incidence of individual TEAEs was observed in the bifeprunox groups. TEAEs with a slightly higher (≧2% difference) incidence in the 30 mg bifeprunox group compared with the 20 mg bifeprunox group included fatigue, dizziness, and sedation. The percentage of subjects with at least one severe TEAE was comparable in the bifeprunox treatment groups and the placebo group (9% to 12%) and slightly less in the olanzapine treatment group (6%). There was no clear indication of a dose-related increase in the incidence of severe TEAEs in the bifeprunox groups for any event. Special interest TEAEs were defined prior to database lock and included the following events: suicide, suicide attempt, events related to sexual dysfunction, syncope, vasovagal attack, and postural hypotension. A total of 58 subjects (10%) overall reported at least one TEAE of special interest during the study. The total number of subjects with at least one special interest TEAE was higher in the bifeprunox treatment groups (30 mg bifeprunox: 20 subjects, 13%; 20 mg bifeprunox: 17 subjects, 11%) compared with the placebo (11 subjects, 7%) or olanzapine (10 subjects, 7%) groups. The majority of TEAEs of special interest occurred in ≦1% of subjects in any treatment group. The most commonly reported TEAE of special interest was dizziness which had a slightly higher incidence in the bifeprunox treatment groups compared with the other two groups (30 mg bifeprunox: 15 subjects, 10%; 20 mg bifeprunox: 13 subjects, 8%; placebo: nine subjects, 6%; olanzapine: eight subjects, 5%). Other special interest TEAEs occurring in at least two subjects within a treatment group included syncope vasovagal (30 mg bifeprunox: two subjects) and orthostatic hypotension (30 mg bifeprunox: three subjects).

The percentage of subjects with at least one SAE was least in the olanzapine treatment group (six subjects, 4%) followed by the bifeprunox groups (20 mg: 15 subjects, 10%; 30 mg: 12 subjects, 8%), with the highest incidence of SAEs noted in the placebo group (20 subjects, 13%). The most commonly reported SAEs were psychotic disorder (4% of subjects overall) and schizophrenia (2% overall). The incidence of these SAEs was similar or lower in the bifeprunox groups compared with the placebo group. Two subjects in the 30 mg bifeprunox group had a SAE of syncope vasovagal compared with no subjects in the other treatment groups. Apart from this possible exception, there were no other notable indications of a doserelated increase in the incidence of any other SAE observed for the bifeprunox groups. The percentage of subjects who discontinued study medication due to an AE was greatest in the placebo group (11%) followed by the bifeprunox groups (8% each) and the olanzapine treatment group (6%). The percentage of subjects with at least one AE that led to study termination was greatest in the placebo group (12%) with comparable percentages of subjects having at least one AE leading to study termination in the 20 mg bifeprunox (8%), 30 mg bifeprunox (7%), and olanzapine (6%) treatment groups. The most common (reported by >2% of subjects in any treatment group) AEs that led to discontinuation of study medication were psychotic disorder and schizophrenia. There was no clear trend of a dose-related increase in the incidence of AEs leading to discontinuation of study medication in the bifeprunox groups. Evaluation of laboratory, vital sign and ECG findings did not raise any unexpected safety concerns. Subjects in the bifeprunox group exhibited a decrease in prolactin compared with subjects in the placebo and olanzapine groups. The incidence of markedly abnormal decreases in body weight was comparable among bifeprunox treatment and placebo groups (5% to 6%) and was lower in the olanzapine treatment group (<1%). The incidence of markedly abnormal increases in body weight was comparable in the bifeprunox and placebo groups (1% to 3%) and much higher in the olanzapine (19%) group. There were no notable differences between the treatment groups in changes from Baseline to Endpoint in BAS, SAS, or AIMS scores. Use of anticholinergic medication for subjects treated with bifeprunox was similar to that of subjects on placebo.

DISCUSSION AND CONCLUSION: This was a treatment period six-week randomized, double-blind, fixed-dose, placebo-controlled, parallel group, multi-center study of the efficacy, tolerability and safety of bifeprunox with olanzapine as an active reference in the treatment of 604 subjects with schizophrenia. The study was conducted at 32 centers in the United States (26), Colombia (3), and India (5).

This was a valid study evidenced by the results demonstrating statistically significant differences between the active control olanzapine, and placebo treatment for the primary efficacy parameter.

Both the 20 mg and 30 mg treatment groups showed improvements over Baseline at Endpoint but did not demonstrate efficacy as compared to the placebo group with respect to the primary and key secondary efficacy parameters. However, the 20 mg bifeprunox group showed notable improvement over the placebo group for the change from Baseline to Endpoint in a secondary efficacy parameter, CGI improvement score (nominal p=0.027). Additionally in one other secondary efficacy parameter, the difference between the 20 mg bifeprunox group and the placebo group approached being notable (p=0.061) for PANSS-20%-responder rate. However, these occurrences of notable and nearly notable differences among the secondary efficacy parameters do not exceed what is expected to happen by chance, i.e., in 5% of the treatment comparisons. In all other secondary and key secondary parameters, neither bifeprunox treatment group showed notable improvement over the placebo group for any efficacy endpoint.

Olanzapine at a dose of 15 mg was used as an active reference in this study. In general, the magnitude of the improvements seen in the bifeprunox dose groups was higher than those seen in the placebo group, but lower than those seen in the olanzapine group for most efficacy endpoints.

In contrast, in the large study in Clinical Study One, the 20 mg bifeprunox dose was effective in reducing both positive and negative symptoms of schizophrenia and lessening overall psychopathology compared to placebo as shown by statistically significant comparisons from the analysis of PANSS total and subscale scores.

In the present study, using the observed values analysis of change from Baseline to Week 6 in the PANSS total score, results were more similar between the 20 mg bifeprunox (−24.42 [15.6]), 30 mg bifeprunox (−24.56 [17.02]), and olanzapine (−29.11 [16.88]) groups, however the placebo group also demonstrated similar results (−22.29 [19.14]). This indicates that subjects who stayed in the study for six weeks responded well to their treatment regimen.

Bifeprunox was well tolerated at both dose levels. The rate of withdrawal due to adverse events was lower in the bifeprunox groups compared with the placebo group. Adverse events appearing more frequently in bifeprunox treated subjects than in placebo subjects were mainly gastrointestinal in nature and mild to moderate in severity. Only two subjects (in the 20 mg bifeprunox group) discontinued study medication due to gastrointestinal AEs (one subject discontinued due to nausea, one subject discontinued due to nausea and vomiting). The percentage of subjects with at least one SAE was lower in the bifeprunox groups (7% to 8%) compared with the placebo group (13%). The most commonly reported SAEs were psychotic disorder (4% overall) and schizophrenia (1% overall). The incidence of these SAEs was similar or lower in the bifeprunox groups compared with the placebo group. Two subjects in the 30 mg bifeprunox group had a SAE of vasovagal syncope compared with no subjects in the other treatment groups. Apart from this possible exception, there were no other notable indications of a dose-related increase in the incidence of any other SAE for the bifeprunox groups.

Evaluation of laboratory, vital sign, and ECG findings did not raise any unexpected safety concerns. Bifeprunox was associated with a decrease in prolactin consistent with what has been seen in previous studies and expected on the basis of the partial dopamine agonistic profile of the drug, and was not associated with any AEs. In contrast, increases in prolactin were observed in the placebo and olanzapine groups.

Small decreases in mean body weight were noted in the bifeprunox groups while an increase in weight was noted for the olanzapine group. The incidence of markedly abnormal decreases in body weight was comparable among bifeprunox treatment and placebo groups (5% to 6%) and was lower in the olanzapine treatment group (□1%). The incidence of markedly abnormal increases in body weight was comparable in the bifeprunox and placebo groups (1% to 3%) and much higher in the olanzapine (19%) group. These findings are consistent with what has been observed in other bifeprunox studies and are noteworthy given that weight gain has been problematic for atypical antipsychotics and is associated with an increased risk of cardiovascular disease and diabetes mellitus.

There were no notable differences between treatment groups on measures of abnormal movement (BAS, SAS, or AIMS scores). Use of anticholinergic medication for subjects treated with bifeprunox was similar to that of subjects on placebo. The incidence of extrapyramidal disorder was low in all groups (bifeprunox groups: <1% to 3%; olanzapine: 1%; placebo: 3%). The 20 mg and 30 mg doses of bifeprunox did not show statistically significantly greater improvement compared with placebo for most efficacy endpoints. Better CGI improvement scores were seen in the 20 mg (but not 30 mg) bifeprunox group compared with the placebo group.

Nausea, vomiting, and constipation were the more notable AEs relative to placebo. Overall, 20 mg and 30 mg doses of bifeprunox were safe and well tolerated by subjects with schizophrenia.

### Example 2d

### Clinical Study Four

PRIMARY OBJECTIVE: to investigate whether 6 months of bifeprunox treatment is superior to treatment with placebo in patients with chronic schizophrenia, using the time to deterioration from randomization as the primary outcome measure. SECONDARY OBJECTIVE: to investigate whether the acute effect after 6 weeks of bifeprunox treatment is superior to treatment with placebo, using the change from baseline in Positive and Negative Syndrome Scale (PANSS) total score as the outcome measure.

OTHER SECONDARY OBJECTIVE: To evaluate the long-term safety and tolerability of bifeprunox versus placebo.

METHODOLOGY: This study was a multinational, multicenter, randomized, double-blind, parallel-group, placebo-controlled, fixed-dose study. The study consisted of a 3- to 6-day antipsychotic-free run-in period, after which patients were randomised to 6 months of double-blind treatment with fixed doses of bifeprunox (20 mg/day (BX20) or 30 mg/day (BX30)) or placebo (PBO). Patients allocated to the BX groups were up-titrated from 0.25 mg/day over 7 days (BX20) or 8 days (BX30), and then continued on these doses for the remainder of the study. Efficacy assessments were made at baseline (except CGI-I) and at Weeks 1, 2, 4, 6, and 9, and at Months 3, 4, 5, and 6. Safety assessments were performed at screening, during treatment, and at the end of the study. At predetermined time points, blood samples were obtained for drug concentration analysis of BX and its major metabolites (3′- and 4′-sulfate conjugates of BX), and pharmaco-economic assessments were performed.

Number of Patients Planned and Analyzed: There were a total of 495 patients that were planned for enrolment: 165 in each treatment group.

Diagnosis and Main Inclusion Criteria: Patients with a primary diagnosis of schizophrenia, according to DSM-IV-TR criteria, for more than 2 years, who: had a PANSS total score ≧60 and a CGI-S score ≧4 (moderately ill) at screening and baseline; had PANSS items P7 (hostility) and G8 (uncooperativeness) scores ≦4 (moderate) at screening and baseline; were between 18 and 65 years of age (extremes included); were inpatients, partially hospitalized, or outpatients followed up in a day care program within 90 days; and prior to screening had no modification of antipsychotic medication within 1 month prior to screening.

Investigational Product, Dose and Mode of Administration, Batch Number: Bifeprunox—up-titration over 7 (20 mg) or 8 days (30 mg) to 20 or 30 mg once daily; encapsulated tablets, orally.

Reference Thera Dose and Mode of Administration: Placebo—encapsulated tablets, orally.

Efficacy Results: The primary efficacy variable was the time to deterioration and the analysis was based on the FAS. The primary efficacy analysis rejected the hypothesis of equal time to deterioration of schizophrenia in the three treatment groups (Cox model, p=0.008). Subsequent pairwise comparisons of each of the BX groups and the PBO group showed that patients in the BX groups had a statistically significantly longer time to deterioration (i.e., there was an increase of the time to deterioration) of schizophrenia than patients in the PBO group (BX20: p=0.008 and BX30: p=0.006). The proportion of patients who deteriorated was 59% in the PBO group, 41% in the BX20 group, and 38% in the BX30 group, The Cox proportional hazards model gave an estimated hazard ratio of 0.66 (BX20) and 0.65 (BX30) relative to PBO; that is, the risk of deterioration was approximately 1.5 times higher for patients in the PBO group than for patients in the BX20 or BX30 groups. Bifeprunox was also statistically significantly superior to PBO in the analysis of time to deterioration based on the PPS. Since most of the patients in the PPS participated for most of the study, the results were very close to the results of the primary analysis, both for the estimated hazard ratios and the p-values obtained. This illustrates the robustness of the conclusion of the primary efficacy analysis. The secondary efficacy variable was the PANSS total score at Week 6. The adjusted mean change from baseline to Week 6 in PANSS total score (FAS, LOCF) for each BX group (BX20: −4.0; BX30: −2.7) was statistically significantly greater than that for the PBO group (1.1) (BX20: p=0.002; BX30: p=0.017) according to Hochberg's Step-up Method.

For the development over time in PANSS total scores, PANSS positive and general psychopathology subscale scores, BPRS total scores, and BPRS psychosis cluster scores (all FAS, LOCF), the same general pattern was seen: initially (until Weeks 6 or 9), the mean scores decreased and subsequently they stabilized in both BX groups, whereas in the PBO group, the mean scores decreased until Weeks 2 or 4, after which the scores increased steadily. For each of these variables, pairwise comparisons of each of the BX groups with the PBO group for the FAS using the LOCF (ANCOVA) showed that treatment with BX (either dose) was generally statistically significantly superior to treatment with PBO from Week 6 or 9 onwards.

The mean PANSS negative subscale scores (FAS, LOCF) decreased in both BX groups until Week 6, after which the scores stabilized. In the PBO group, the scores decreased until Week 4, after which the scores tended to increase. In the per-visit LOCF analysis (FAS, ANCOVA) of the mean PANSS negative subscale scores, treatment with both BX doses was statistically significantly superior to that with PBO at all time points, except at Weeks 2 and 4 for the BX30 group.

For all treatment groups, the mean CDSS total scores (FAS, LOCF) decreased initially (baseline to Week 2), after which the scores either remained stable (both BX groups) or increased (PBO group) over time. In the per-visit LOCF analysis (FAS, ANCOVA) of the mean CDSS total scores, there were no statistically significant differences between either of the BX groups and the PBO group at any time point. This result should be seen in the context that the baseline CDSS scores were low, reflecting a low level of depressive symptomatology.

The mean CGI-S scores (FAS, LOCF) decreased in both BX groups until Week 6, after which the scores stabilized. In the PBO group, the mean CGI-S scores decreased until Week 4, after which the scores tended to increase. In the per-visit LOCF analysis (FAS, ANCOVA) of the mean CGI-S scores, the BX groups were statistically significantly superior to the PBO group at Months 3, 4, 5, and 6 for BX20 and Months 5 and 6 for BX30.

The mean CGI-I scores (FAS, LOCF) decreased in both BX groups until Week 6, after which the scores stabilized. In the PBO group, the scores decreased minimally until Week 2, after which the scores increased steadily. In the per-visit LOCF analysis (FAS, ANCOVA) of the mean CGI-I scores, both BX doses were statistically significantly superior to PBO from Week 6 onwards.

For the small number of patients who fulfilled the criteria for predominantly negative symptoms (PBO: 25 patients; BX20: 32 patients; BX30: 36 patients), the adjusted mean change from baseline in PANSS total scores (all groups) and in PANSS positive subscale scores (PB0 and BX20) generally followed the same patterns and were within the same range as those for the overall population. In contrast, the adjusted mean change from baseline in PANSS positive subscale score for patients treated with BX30 followed the same pattern over time as for the overall population but the change was larger. The adjusted mean change from baseline in PANSS negative subscale scores (all groups) followed the same pattern over time as the overall population; however, the mean change was generally twice as large in this population relative to the overall population. In the per-visit LOCF analysis (ANCOVA) of PANSS total scores, PANSS positive subscale scores, and PANSS negative subscale scores, BX20 was not statistically significantly different from PBO at any time points. BX30 was statistically significantly superior to PBO in PANSS total scores from Week 6 onwards and in PANSS positive subscale scores from Week 2, onwards whereas BX30 was not statistically significantly different from PBO from Week 2 onwards in PANSS negative subscale scores.

For the small number of patients who fulfilled the criteria for predominantly depressive symptoms (PBO: 18 patients; BX20: 15 patients; BX30: 22 patients), the PANSS total scores, the PANSS positive subscale scores, the PANSS negative subscale scores, and the CDSS total scores followed generally similar overall patterns as those for the overall population. In the per-visit LOCF analysis (ANCOVA) of the PANSS total scores, PANSS positive subscale scores, PANSS negative subscale scores, and CDSS total scores, there were no statistically significant differences between either of the BX groups and the PBO group at any time point in this sub-population, probably due to the low number of patients and the low level of depressive symptoms present at baseline.

The proportion of patients with at least a 25% reduction in PANSS total score (FAS, LOCF) in the BX20 group was statistically significantly larger than that in the PBO group from Week 9 onwards, whereas the proportion of responders in the BX30 group was statistically significantly larger than that in the PBO group at Month 5 only.

A small proportion (0% to 8%) of patients (irrespective of treatment) had a reduction ≧35%, ≧45%, or ≧55% in PANSS total score. There was no trend between or within treatment groups with respect to the distribution in the proportion of patients with a specific reduction.

The proportion of patients with a CGI-I score ≦2 (FAS, LOCF) at Week 6 and Month 6 was larger in the BX groups (Week 6: BX20 17%; BX30 19%; Month 6: BX20 22%; BX30 20%) than in the PBO group (Week 6; 11%; Month 6: 9%). The differences between the BX groups and the PBO group were statistically significant (p<0.05) from Week 9 onwards (BX20) and from Week 9 onwards except Month 3 (BX30).

The mean total cholesterol and mean LDL calculated decreased from baseline to Month 6 in all groups (except non-fasting PBO patients) irrespective of treatment and fasting/non-fasting condition. The mean VLDL calculated and triglycerides decreased from baseline to Month 6 in all groups (except non-fasting BX30 patients) irrespective of treatment and fasting/non-fasting condition. The mean HDL increased from baseline to Month 6 in all groups irrespective of treatment and fasting/non-fasting condition. The adjusted mean HDL cholesterol values increased from baseline to Month 6 in all three treatment groups irrespective of fasting/nonfasting condition (PBO: 0.04/0.06 (fasting/non-fasting); BX20-0.07/0.08; BX30: 0.07/0.08 mmol/L). There were no statistically significant differences between either of the BX groups and the PBO group.

The adjusted mean triglycerides values decreased from baseline to Month 6 in all three treatment groups irrespective of fasting/non-fasting condition (PBO: −0.06/−0.22 (fasting/non-fasting); BX20: −0.16/−0.21; BX30: −0.37/−0.03 mmol/L). There were no statistically significant differences between either of the BX groups (except BX30 (fasting)) and the PBO group.

The adjusted mean fasting glucose values increased from baseline to Month 6 in all three treatment groups (PBO: 0.10; BX20: 0.13; BX30: 0.09 mmol/L) and there were no statistically significant differences between either of the BX groups and the PBO group.

The adjusted mean weight change from baseline to Month 6 (APTS, OC, ANCOVA) was −0.8 kg in the PBO group, −0.3 kg in the BX20 group, and −0.5 kg in the BX30 group. The adjusted mean weight decreases in the BX20 and the BX30 group were not statistically significantly different from that in the PBO group.

In all treatment groups, patients lost weight irrespective of whether they had nausea and/or vomiting, although those patients who also had nausea and/or vomiting had a greater weight decrease (PBO: −0.6 versus −1.9 kg; BX20: −1.0 versus −1.9 kg; BX30: −1.1 versus −2.3 kg).

There was no treatment effect of BX (either dose) on the patients' status of metabolic syndrome during the study. Approximately 75% (range: 70% to 80%) of the patients did not have metabolic syndrome at baseline or at the end of the study. There were no statistically significant treatment differences in the patients' status of metabolic syndrome.

There were no clinically relevant changes within or differences between treatment groups in clinical laboratory values, vital signs, metabolic syndrome, or ECG parameters.

CONCLUSIONS: One conclusion of this study is that both doses of bifeprunox (20 mg/day and 30 mg/day) prevented deterioration of schizophrenia statistically significantly better than placebo over the treatment period of 6 months. For patients with chronic, stable schizophrenia, the baseline condition was significantly better maintained with BX (both doses) than with PBO either after 6 weeks of treatment or after long-term treatment. A comparison of the safety profiles of bifeprunox and placebo showed a higher incidence of adverse events related to the gastrointestinal system and dizziness in the bifeprunox groups relative to the placebo group. The incidence of nausea and vomiting leading to withdrawal from the study and abnormal movements was higher in the BX30 group than in the BX20 group. A favorable metabolic profile (based on weight changes, blood lipids and the presence/absence of metabolic syndrome) was seen for bifeprunox.

### Results Related to Clinical Studies One-Four PANSS Total Score

CLINICAL STUDY ONE: As provided in Table 1, the mean change (S.D.) from Baseline to Endpoint in PANSS total score was −9.7 (17.5) for the bifeprunox 5 mg group, −5.0 (18.3) for the bifeprunox 10 mg group, −11.3 (17.0) for the bifeprunox 20 mg group, −5.3 (16.3) for the placebo group, and −15.7 (14.9) for the risperidone group. The treatment effect values corresponding to the difference between bifeprunox and placebo mean change from Baseline at Endpoint (LOCF) were −4.1, 0.6, and −5.8 for the bifeprunox 5 mg, 10 mg, and 20 mg groups respectively. From the pairwise comparisons, a statistically significantly greater decrease at Endpoint (LOCF) was seen for the 20 mg bifeprunox group versus placebo (p-value adjusted for multiple comparisons, p=0.031). No significant treatment group differences were seen for the 5 mg bifeprunox and 10 mg bifeprunox treatment groups compared to placebo, respectively.

TABLE 1
Change from Baseline in PANSS Total Score-Last Observation
Carried Forward at Each Visit Intent-to-Treat Population.

| | | Treatment Group | | | | |
|---|---|---|---|---|---|---|
| | | Bifeprunox | Bifeprunox | Bifeprunox | | Risperidone |
| | Statistic | 5 mg | 10 mg | 20 mg | Placebo | 6 mg |
| Total Number of | N | 110 | 118 | 111 | 114 | 116 |
| Subjects in the ITT | | | | | | |
| Population | | | | | | |
| Baseline | n | 110 | 118 | 111 | 114 | 116 |
| | Mean (S.D.) | 91.1 (11.1) | 93.5 (11.6) | 92.9 (12.2) | 92.1 (12.3) | 90.9 (11.6) |
| | Median | 90 | 93 | 92 | 91 | 90 |
| | Min-Max | 73-120 | 67-130 | 70-129 | 70-120 | 70-136 |
| Change from Baseline | | | | | | |
| Week 1 | n | 110 | 118 | 110 | 114 | 116 |
| | Mean (S.D.) | −3.9 (13.1) | −2.9 (11.7) | −6.3 (11.5) | −4.0 (10.1) | −8.2 (11.1) |
| | Median | −4 | −1 | −5 | −3 | −7 |
| | Min-Max | −50-76 | −47-30 | −53-20 | −40-24 | −51-13 |
| | P-Value (adj) | 1.000 | 1.000 | 0.342 | | |
| Week 2 | n | 110 | 118 | 110 | 114 | 116 |
| | Mean (S.D.) | −6.7 (14.9) | −4.2 (14.6) | −9.0 (14.0) | −5.1 (13.6) | −13.1 (13.9) |
| | Median | −7 | −3 | −8 | −5 | −12 |
| | Min-Max | −62-76 | −47-37 | −55-30 | −44-33 | −69-14 |
| | P-Value (adj) | 0.696 | 1.000 | 0.113 | | |
| Week 3 | n | 110 | 118 | 111 | 114 | 116 |
| | Mean (S.D.) | −7.7 (16.1) | −4.6 (17.4) | −11.2 (14.5) | −6.0 (15.5) | −15.3 (14.1) |
| | Median | −7 | −3 | −11 | −6 | −13 |
| | Min-Max | −65-76 | −54-41 | −59-33 | −46-36 | −64-14 |
| | P-Value (adj) | 0.726 | 1.000 | 0.047* | | |
| Week 4 | n | 110 | 118 | 111 | 114 | 116 |
| | Mean (S.D.) | −8.7 (17.1) | −4.1 (17.2) | −11.2 (15.9) | −6.5 (16.0) | −14.4 (14.5) |
| | Median | −7 | −5 | −11 | −6 | −13 |
| | Min-Max | −65-76 | −55-41 | −56-33 | −41-36 | −66-14 |
| | P-Value (adj) | 0.625 | 1.000 | 0.097 | | |
| Endpoint LOCF | n | 110 | 118 | 111 | 114 | 116 |
| | Mean (S.D.) | −9.7 (17.5) | −5.0 (18.3) | −11.3 (17.0) | −5.3 (16.3) | −15.7 (14.9) |
| | Median | −8 | −4 | −11 | −6 | −14 |
| | Min-Max | −66-76 | −57-41 | −63-33 | −52-37 | −57-15 |
| | Trt Effect | −4.1 | 0.6 | −5.8 | | |
| | Trt Effect CI | (−9.2, 0.9) | (−4.4, 5.5) | (−11.1, −0.4) | | |
| | P-Value (adj) | 0.128 | 1.000 | 0.031* | | |

Note:
*Statistically significant after adjusting for multiple comparisons using the Step Down Dunnett's procedure with an overall type I Error of 0.0499. P-values that favor placebo are adjusted to 1.000. Confidence intervals were also derived in accordance with the Step Down Dunnett's procedure.
Note:
Pairwise comparisons versus placebo are based on an ANCOVA model with treatment (exluding risperidone) and pooled center as fixed factors and baseline PANSS total score as a covariate.
Note:
No PANSS scores were recorded at Week 1 or Week 2 for Subject 11259 in the bifeprunox 20 mg group.

CLINICAL STUDY TWO: As provided in Table 2, the mean change (SD) from Baseline to Endpoint in PANSS total score was −13.5 (20.1) for the 30 mg bifeprunox group, −10.3 (20.5) for the 40 mg bifeprunox group, −7.7 (19.2) for the placebo group, and −19.7 (19.3) for the risperidone group (as indicated in the below Table). The treatment effect values (for mean change from Baseline at Endpoint [LOCF]) corresponding to the difference between bifeprunox and placebo were: −5.9 for the 30 mg bifeprunox group and −3.2 for the 40 mg bifeprunox group. A statistically significant treatment group difference was seen for the 30 mg bifeprunox treatment group compared to placebo at Endpoint based on the Hochberg adjusted p-value (p=0.020). Differences between 30 mg bifeprunox and placebo treatment groups were also noted at Week 2 through Week 4 from the nominal p-values (p≦0.004 at each of Week 2 through Week 4). In addition, the treatment effect increased consistently over the 6 weeks of study (range of effect: −5.6 at week 2 and −6.3 at week 4). No statistically significant difference between 40 mg bifeprunox group and placebo group was evident at Endpoint based on the Hochberg adjusted p-value.

TABLE 2
Change from Baseline in PANSS Total Score Last Observation
Carried Forward at Each Visit Intent-to-Treat Population.

| | | Treatment Group | | | |
|---|---|---|---|---|---|
| | | Bifeprunox | Bifeprunox | | Risperidone |
| | Statistic | 30 mg | 40 mg | Placebo | 6 mg |
| Total Number of | N | 140 | 141 | 144 | 151 |
| Subjects in the ITT | | | | | |
| Population | | | | | |
| Baseline | n | 140 | 141 | 144 | 151 |
| | Mean (SD) | 94.8 (11.5) | 92.7 (11.2) | 93.9 (11.9) | 92.7 (11.3) |
| | Median | 94 | 92 | 94 | 91 |
| | Min, Max | 71, 120 | 71, 119 | 59, 124 | 72, 124 |
| Change from Baseline | n | 140 | 139 | 143 | 149 |
| Week 1 | Mean (SD) | −6.6 (11.8) | −6.7 (11.5) | −5.4 (11.6) | −8.7 (10.2) |
| | Median | −5 | −6 | −5 | −9 |
| | Min, Max | −55, 26 | −50, 29 | −60, 31 | −39, 29 |
| | P-value | 0.402 | 0.259 | | |
| Week 2 | n | 140 | 140 | 144 | 151 |
| | Mean (SD) | −11.6 (15.5) | −8.9 (15.6) | −6.0 (216.0) | −14.8 (14.2) |
| | Median | −10 | −8 | −6 | −15 |
| | Min, Max | −56, 34 | −50, 28 | −51, 40 | −49, 28 |
| | P-value | 0.002 | 0.055 | | |
| Week 3 | n | 140 | 140 | 144 | 151 |
| | Mean (SD) | −13.3 (17.8) | −10.0 (17.8) | −7.3 (18.3) | −17.1 (15.3) |
| | Median | −10 | −10 | −7 | −18 |
| | Min, Max | −60, 34 | −72, 28 | −72, 40 | −57, 28 |
| | P-value | 0.003 | 0.092 | | |
| Week 4 | n | 140 | 140 | 144 | 151 |
| | Mean (SD) | −13.6 (18.9) | −9.2 (18.8) | −7.4 (19.1) | −19.2 (17.0) |
| | Median | −10 | −7 | −6 | −20 |
| | Min, Max | −64, 34 | −72, 28 | −74, 40 | −74, 28 |
| | P-value | 0.004 | 0.259 | | |
| Week 6/Endpoint | N | 140 | 141 | 144 | 151 |
| | Mean (SD) | −13.5 (20.1) | −10.3 (20.5) | −7.7 (19.2) | −19.7 (19.3) |
| | Median | −10 | −7 | −7 | −21 |
| | Min, Max | −71, 34 | −76, 37 | −74, 40 | −76, 46 |
| | Trt Effect | −5.9 | −3.2 | | |
| | 95% CI | −10.3, −1.4 | −7.7, 1.2 | | |
| | 97.5% CI | −11.0, −0.8 | −8.3, 1.9 | | |
| | P-value (Raw) | 0.010 | 0.156 | | |
| | P-value (Adj) | 0.020* | 0.156 | | |

*Significant at the 0.050 level according to the Hochberg procedure.
Note:
Treatment comparisons versus placebo were based on an ANCOVA model with treatment (excluding risperidone) and pooled center as fixed factors and Baseline PANSS total score as a covariate.
Note:
Significance at Week 6/Endpoint for multiple comparisons was evaluated according to the Hochberg procedure.
Note:
P-values presented for Week 1 through Week 4 are for descriptive purposes only.
Trt = Treatment,
Adj = Adjusted.

CLINICAL STUDY THREE. The mean change (SD) from Baseline to Endpoint in PANSS total score was −13.8 (19.9) for the 20 mg bifeprunox group, −13.1 (20.2) for the 30 mg bifeprunox group, −10.7 (19.4) for the placebo group, and −22.0 (18.2) for the olanzapine group (Table 3). The treatment effect values (for mean change from Baseline at Endpoint [LOCF]) corresponding to the difference between bifeprunox and placebo were: −3.5 for the 20 mg bifeprunox group and, −2.2 for the 30 mg bifeprunox group. No statistically significant treatment group differences were seen for the 20 mg or 30 mg biteprunox treatment groups compared to the placebo group based on the Hochberg adjusted p-values. Similarly no notable differences were observed between the placebo group and either of the two bifeprunox dose groups at any other time point during the study (Week 1 through Week 4). When data were analyzed only for those subjects having the same rater for each visit, differences in mean change (SD) from Baseline to Endpoint in PANSS total score (LOCF) were similar to those observed in the primary analysis. The difference of the PANSS total score between olanzapine and placebo was analyzed in accordance with sensitivity analyses. These results showed that olanzapine was notably different from placebo (p<0.001).

TABLE 3
Change from Baseline in PANSS Total Score Last Observation
Carried Forward at Each Visit Intent-to-Treat Population.

| | | Treatment Group | | | |
|---|---|---|---|---|---|
| | | Bifeprunox | Bifeprunox | | Olanzapine* |
| | Statistic | 20 mg | 30 mg | Placebo | 15 mg |
| Total Number of | N | 149 | 148 | 145 | 146 |
| Subjects in | | | | | |
| the ITT Population | | | | | |
| Baseline | n | 149 | 148 | 145 | 146 |
| | Mean (SD) | 93.9 (11.0) | 95.6 (10.6) | 94.8 (11.3) | 96.4 (11.7) |
| | Median | 94 | 96 | 94 | 96 |
| | Min, Max | 71, 119 | 73, 121 | 70, 119 | 70, 122 |
| Change from | | | | | |
| Baseline | | | | | |
| Week 1 | n | 149 | 146 | 144 | 145 |
| | Mean (SD) | −5.6 (11.7) | −6-1 (12.3) | −6.4 (11.0) | −9.3 (11.2) |
| | Median | −5 | −6 | −5 | −6 |
| | Min, Max | −46, 49 | −44, 54 | −70, 13 | −67, 8 |
| | P-value | 0.618 | 0.790 | | |
| Week 2 | n | 149 | 147 | 145 | 146 |
| | Mean (SD) | −10.2 (14.8) | −8.5 (15.6) | −10.4 (14.7) | −15.1 (13.6) |
| | Median | −10 | −9 | −9 | −12 |
| | Min-Max | −57, 49 | −57, 65 | −57, 28 | −56, 19 |
| | P-value | 0.943 | 0.257 | | |
| Week 3 | n | 149 | 148 | 145 | 146 |
| | Mean (SD) | −12.6 (16.9) | −11.1 (18.0) | −11.9 (16.9) | −19.3 (15.8) |
| | Median | −13 | −11 | −12 | −18 |
| | Min, Max | −66, 49 | −59, 65 | −64, 36 | −69, 19 |
| | P-value | 0.594 | 0.655 | | |
| Week 4 | n | 149 | 148 | 145 | 146 |
| | Mean (SD) | −13.3 (18.7) | −12.0 (18.6) | −11.3 (18.4) | −20.0 (16.5) |
| | Median | −13 | −13 | −8 | −18 |
| | Min, Max | −70, 49 | −62, 65 | −62, 36 | −69, 19 |
| | P-value | 0.280 | 0.817 | | |
| Week 6/ | n | 149 | 148 | 145 | 146 |
| Endpoint LOCF | Mean (SD) | −13.8 (19.9) | −13.1 (20.2) | −10.7 (19.4) | −22.0 (18.2) |
| | Median | −14 | −13 | −8 | −21 |
| | Min, Max | −68, 53 | −71, 65 | −68, 36 | −79, 19 |
| | Trt Effect | −3 5 | −2.2 | | |
| | Trt Effect | −7.8, 0.9 | −6.6, 2.2 | | |
| | 95% CI | | | | |
| | Trt Effect | −8.4, 1.5 | −7.2, 2.8 | | |
| | 97.5% CI | | | | |
| | P-value (Raw) | 0.121 | 0.321 | | |
| | P-value. (Adj) | 0.241 | 0.321 | | |

Note:
Treatment comparisons versus placebo were based on an ANCOVA model with treatment (excluding olanzapine) and pooled center as fixed factors and Baseline PANSS total score as a covariate.
Note:
Significance at Week 6/Endpoint for multiple comparisons was evaluated according to the Hochberg procedure.
Note:
P-values and CIs presented for Week 1 through Week 4, and the raw p-value in Week 6/Endpoint are for descriptive purposes only.
*Olanzapine was excluded from statistical comparisons of treatment groups as data from this treatment group was to be considered in supportive analyses only.
Trt = Treatment,
Adj = Adjusted.

CLINICAL STUDY FOUR: The adjusted mean change from baseline to Week 6 in PANSS total score for each BX group (BX20: −4.0 and BX30: −2.7) was statistically significantly greater than that for the PBO (1.1) group (BX20: p=0.002; BX30: p=0.017), as indicated in Table 4.

TABLE 4
Adjusted Mean Change from Baseline in PANSS Total Score Week 6
(FAS, LOCF, ANCOVA).

| | | | Least Squares | | | Difference to PBO | | | |
|---|---|---|---|---|---|---|---|---|---|
| Treatment | | | Estimates | | | | 95% CI | 95% CI | |
| Group | Days | n | Mean | SE | Mean | SE | Lower | Upper | p-value |
| PBO | 42 | 166 | 1.06 | 1.17 | | | | | |
| BX20 | 42 | 158 | −4.01 | 1.20 | −5.07 | 1.59 | −8.19 | −1.95 | 0.002 |
| BX30 | 42 | 172 | −2.65 | 1.13 | −3.71 | 1.55 | −6.76 | −0.66 | 0.017 |

### PANSS Positive

CLINICAL STUDY ONE: Table 5 presents mean PANSS positive subscale scores at Baseline and the mean change from Baseline by visit using LOCF for the ITT population. The treatment effect values (bifeprunox—placebo) at Endpoint LOCF were −1.1, 0.7, −1.5 for the bifeprunox 5 mg, 10 mg, and 20 mg groups respectively. A statistically significantly greater decrease (unadjusted p=0.037) at Endpoint (LOCF) was seen in PANSS positive subscale score for the comparison of the treatment effect estimates for the bifeprunox 20 mg group and placebo.

TABLE 5
Change from Baseline in PANSS Positive Subscale Score-Last
Observation Carried Forward at Each Visit Intent-to-Treat Population.

| | | Treatment Group | | | | |
|---|---|---|---|---|---|---|
| | | Bifeprunox | Bifeprunox | Bifeprunox | | Risperidone |
| | Statistic | 5 mg | 10 mg | 20 mg | Placebo | 6 mg |
| Total Number of | N | 110 | 118 | 111 | 114 | 116 |
| Subjects in the | | | | | | |
| ITT Population | | | | | | |
| Baseline | n | 110 | 118 | 111 | 114 | 116 |
| | Mean (S.D.) | 24.5 (3.8) | 24.6 (4.0) | 24.9 (3.4) | 24.4 (4.1) | 24.0 (3.8) |
| | Median | 24 | 25 | 25 | 24 | 24 |
| | Min-Max | 18-120 | 16-35 | 18-33 | 15-39 | 16-36 |
| Change from | | | | | | |
| Baseline | | | | | | |
| Week 1 | n | 110 | 118 | 110 | 114 | 116 |
| | Mean (S.D.) | −1.1 (3.4) | −0.6 (3.9) | −1.7 (3.5) | −1.1 (3.2) | −2.7 (3.4) |
| | Median | −1 | −1 | −1 | −1 | −2 |
| | Min-Max | −10-14 | −12-10 | −12-6 | −12-10 | −13-7 |
| | P-value | 0.973 | 0.238 | 0.228 | | |
| Week 2 | n | 110 | 118 | 110 | 114 | 116 |
| | Mean (S.D.) | −2.1 (4.0) | −1.2 (4.7) | −2.7 (4.3) | −2.1 (4.4) | −4.3 (4.2) |
| | Median | −2 | −1 | −3 | −2 | −4 |
| | Min-Max | −12-14 | −12-11 | −15-11 | −14-10 | −17-6 |
| | P-value | 0.868 | 0.068 | 0.388 | | |
| Week 3 | n | 110 | 118 | 111 | 114 | 116 |
| | Mean (S.D.) | −2.6 (4.7) | −1.1 (5.5) | −3.3 (4.4) | −2.3 (4.9) | −5.0 (4.6) |
| | Median | −3 | −1 | −3 | −3 | −5 |
| | Min-Max | −15-14 | −14-16 | −13-11 | −16-10 | −16-5 |
| | P-value | 0.621 | 0.053 | 0.110 | | |
| Week 4 | n | 110 | 118 | 111 | 114 | 116 |
| | Mean (S.D.) | −3.0 (5.0) | −1.1 (5.6) | −3.5 (4.8) | −2.3 (5.1) | −4.9 (4.6) |
| | Median | −4 | −1 | −3 | −2 | −4 |
| | Min-Max | −15-14 | −13-16 | −17-11 | −13-10 | −17-5 |
| | P-value | 0.328 | 0.058 | 0.084 | | |
| Endpoint LOCF | n | 110 | 118 | 111 | 114 | 116 |
| | Mean (S.D.) | −3.2 (5.2) | −1.4 (6.1) | −3.5 (5.1) | −2.0 (5.2) | −5.3 (4.8) |
| | Median | −3 | −1 | −3 | −2 | −5 |
| | Min-Max | −21-14 | −18-16 | −18-11 | −13-14 | −17-5 |
| | Trt Effect | −1.1 | 0.7 | −1.5 | | |
| | Trt Effect CI | (−2.6, 0.3) | (−0.7, 2.1) | (−2.9, −0.1) | | |
| | P-value | 0.111 | 0.339 | 0.037* | | |

*Significant at the 0.050 level.
Note:
Pairwise comparisons versus placebo are based on an ANCOVA model with treatment (excluding risperidone) and pooled center as fixed factors and baseline PANSS positive subscale score as a covariate.
Note:
No PANSS scores were recorded at Week 1 or Week 2 for Subject 11259 in the bifeprunox 20 mg group.

For the observed values analysis of change from Baseline similar trends to the PANSS total score were seen in the change from Baseline in PANSS positive subscale score in the bifeprunox treatment groups over time. The pairwise comparisons between the bifeprunox and placebo groups were not statistically significant.

CLINICAL STUDY TWO: Table 6 presents PANSS Positive Symptom subscale scores at Baseline and the change from Baseline by visit using LOCF for the ITT population. The mean change (SD) from Baseline to Endpoint in PANSS Positive Symptom subscale scores was −4.5 (6.6) for the 30 mg bifeprunox group, −4.2 (6.9) for the 40 mg bifeprunox group, −2.5 (6.0) for the placebo group, and −7.2 (6.6) for the risperidone group. The treatment effect values (bifeprunox—placebo) at Endpoint LOCF were: −1.9 for the 30 mg bifeprunox group, and −1.7 for the 40 mg bifeprunox group. SEP1 did not yield a significant result, and therefore SEP2 and SEP3 were not evaluated (see Section 7.4.1.1). However, a notable difference between the bifeprunox 30 mg treatment group and placebo was observed at Endpoint (nominal p=0.01). Notable differences between 30 mg bifeprunox and placebo treatment groups were also observed at Week 2 through Week 4 (p≦0.006).

Similarly, notable differences between the bifeprunox 40 mg treatment group and placebo were observed at Endpoint (p=0.020). Differences relative to placebo were also observed at Week 1 through Week 3 (p≦0.013).

TABLE 6
Change from Baseline in PANSS Positive Symptom Subscale Score
Last Observation Carried Forward at Each Visit Intent-to-Treat Population.

| | | Treatment Group | | | |
|---|---|---|---|---|---|
| | | Bifeprunox | Bifeprunox | | Risperidone |
| | Statistic | 30 mg | 40 mg | Placebo | 6 mg |
| Total Number of | N | 140 | 141 | 144 | 151 |
| Subjects in the ITT | | | | | |
| Population, n | | | | | |
| Baseline | N | 140 | 141 | 144 | 151 |
| | Mean (SD) | 25.4 (4.0) | 25.4 (4.0) | 25.0 (4.2) | 25.3 (3.8) |
| | Median | 25 | 25 | 25 | 25 |
| | Min, Max | 18, 40 | 15, 36 | 17, 37 | 17, 36 |
| Change from | | | | | |
| Baseline | | | | | |
| Week 1 | n | 140 | 139 | 143 | 149 |
| | Mean (SD) | −2.3 (3.6) | −2.8 (3.9) | −1.7 (3.5) | −3.3 (3.6) |
| | Median | −2 | −2 | −2 | −3 |
| | Min, Max | −16, 7 | −15, 6 | −14, 13 | −11, 7 |
| | P-value | 0.192 | 0.013 | | |
| Week 2 | n | 140 | 140 | 144 | 151 |
| | Mean (SD) | −4.1 (5.3) | −3.6 (5.7) | −1.9 (5.0) | −5.3 (4.7) |
| | Median | −3 | −3 | −2 | −5 |
| | Min, Max | −18, 11 | −21, 12 | −18, 13 | −18, 6 |
| | P-value | <0.001 | 0.006 | | |
| Week 3 | n | 140 | 140 | 144 | 151 |
| | Mean (SD) | −4.5 (5.6) | −4.1 (6.3) | −2.3 (5.6) | −6.3 (5.3) |
| | Median | −4 | −4 | −2 | −6 |
| | Min, Max | −19, 11 | −19, 13 | −22, 13 | −21, 6 |
| | P-value | 0.001 | 0.008 | | |
| Week 4 | n | 140 | 140 | 144 | 151 |
| | Mean (SD) | −4.4 (5.9) | −3.7 (6.6) | −2.4 (5.9) | −7.0 (5.6) |
| | Median | −4 | −3 | −2 | −7 |
| | Min, Max | −21, 11 | −21, 13 | −22, 13 | −22, 6 |
| | P-value | 0.006 | 0.076 | | |
| Week 6/Endpoint | n | 140 | 141 | 144 | 151 |
| | Mean (SD) | −4.5 (6.6) | −4.2 (6.9) | −2.5 (6.0) | −7.2 (6.6) |
| | Median | −4 | −4 | −2 | −7 |
| | Min, Max | −19, 11 | −21, 13 | −23, 13 | −22, 18 |
| | Trt Effect | −1.9 | −1.7 | | |
| | 95% CI | −3.4, −0.5 | −3.2, −0.3 | | |
| | 97.5% CI | −3.6, −0.3 | −3.4, −0.1 | | |
| | P-value (Raw) | 0.010 | 0.020 | | |

Note:
Treatment comparisons versus placebo were based on an ANCOVA model with treatment (excluding risperidone) and center as fixed factors and Baseline PANSS positive symptom sub-scale score as a covariate.
Note:
p-values presented for Week 1 through Week 4 are for descriptive purposes only.
Trt = Treatment.

CLINICAL STUDY THREE; Table 7 below presents PANSS Positive Symptom subscale scores at Baseline and the change from Baseline by visit using LOCF for the ITT population. The mean change (SD) from Baseline to Endpoint in PANSS Positive Symptom subscale score was −4.3 (6.1) for the 20 mg bifeprunox group, −4.2 (6.8) for the 30 mg bifeprunox group, −3.5 (6.2) for the placebo group, and −7.0 (5.8) for the olanzapine group. The treatment effect values (bifeprunox—placebo) at Endpoint LOCF were: −1.1 for the 20 mg bifeprunox group and, −0.7 for the 30 mg bifeprunox group. No differences with nominal p-values (s 0.05) were observed between the placebo group and either of the bifeprunox dose groups at Week 6/Endpoint or at any other timepoint during the study (Week 1 through Week 4).

TABLE 7
Change from Baseline in PANSS Positive Symptom Subscale Score
Last Observation Carried Forward at Each Visit Intent-to-Treat Population.

| | | Treatment Group | | | |
|---|---|---|---|---|---|
| | | Bifeprunox | Bifeprunox | | Olanzapine |
| | Statistic | 20 mg | 30 mg | Placebo | 15 mg |
| Total Number of | N | 149 | 148 | 145 | 146 |
| Subjects in | | | | | |
| the ITT Population | | | | | |
| Baseline | n | 149 | 148 | 145 | 146 |
| | Mean | 24.6 (3.4) | 25.5 (4.1) | 25.4 (3.9) | 25.3 (3.6) |
| | (SD) | | | | |
| | Median | 25 | 25 | 25 | 25 |
| | Min, Max | 16, 32 | 16, 37 | 17, 35 | 17, 35 |
| Change from | | | | | |
| Baseline | | | | | |
| Week 1 | n | 149 | 146 | 144 | 145 |
| | Mean | −1.6 (3.7) | −2.0 (3.8) | −2.1 (3.7) | −3.0 (3.3) |
| | (SD) | | | | |
| | Median | −2 | −2 | −2 | −2 |
| | Min, Max | −13, 13 | −12, 13 | −20, 9 | −18, 4 |
| | P-value | 0.453 | 0.784 | | |
| Week 2 | n | 149 | 147 | 145 | 146 |
| | Mean | −3.0 (4.7) | −2.8 (5.1) | −3.5 (4.6) | −4.9 (4.0) |
| | (SD) | | | | |
| | Median | −3 | −3 | −3 | −5 |
| | Min, Max | −18, 13 | −15, 15 | −19, 8 | 16, 6 |
| | P-value | 0.654 | 0.244 | | |
| Week 3 | n | 149 | 148 | 145 | 146 |
| | Mean | −3.8 (5.2) | −3.6 (6.0) | −4.1 (5.5) | −6.0 (4.9) |
| | (SD) | | | | |
| | Median | −3 | −4 | −4 | −6 |
| | Min, Max | −19, 13 | −19, 15 | −23, 8 | −20, 6 |
| | P-value | 0.893 | 0.417 | | |
| Week 4 | n | 149 | 148 | 145 | 146 |
| | Mean | −4.2 (5.7) | −4.0 (6.3) | −3.9 (6.0) | −6.4 (5.4) |
| | (SD) | | | | |
| | Median | −4 | −4 | −3 | −7 |
| | Min, Max | −19, 13 | 22, 15 | −25, 9 | −20, 6 |
| | P-value | 0.408 | 0.929 | | |
| Week 6/Endpoint | n | 149 | 14.8 | 145 | 146 |
| | Mean | −4.3 (6.1) | −4.2 (6.8) | −3.5 (6.2) | −7.0 (5.8) |
| | (SD) | | | | |
| | Median | −4 | −5 | −3 | −7 |
| | Min, Max | −19, 14 | −26, 15 | −21, 10 | −23, 6 |
| | Trt Effect | −1.1 | −0.7 | | |
| | Trt Effect | −2.5, 0.3 | −2.1, 0.7 | | |
| | 95% CI | | | | |
| | P-Value | 0.112 | 0.335 | | |

Note:
Treatment comparisons versus placebo were based on an ANCOVA model with treatment (excluding olanzapine) and pooled center as fixed factors and Baseline PANSS positive symptom subscale score as a covariate.
Note:
Significance at Week 6/Endpoint for multiple comparisons was evaluated according to the Hochberg procedure.
Note:
P-values and CIs presented for Week 1 through Week 4, and Week 6/Endpoint are for descriptive purposes only.
Trt = Treatment.

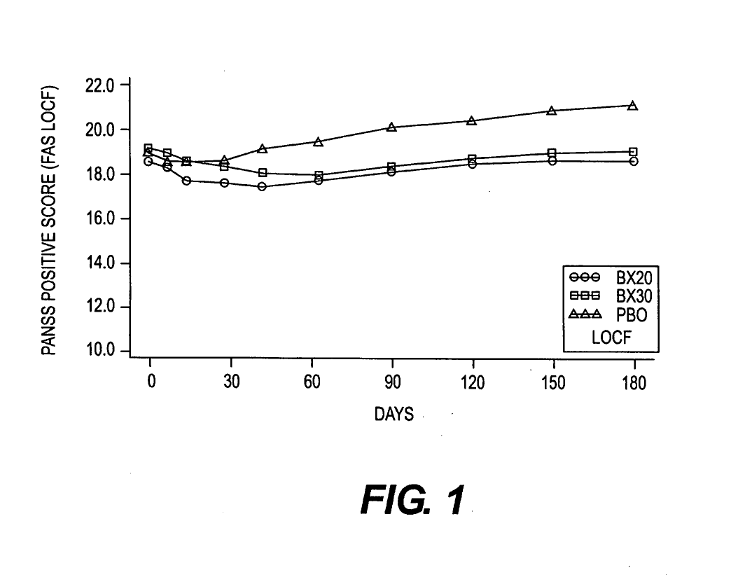

CLINICAL STUDY FOUR: The mean PANSS positive subscale scores (FAS, LOCF) decreased over time in both BX groups until Week 9, after which the scores increased minimally. In the PBO group, the mean PANSS total scores decreased until Week 2, after which the scores increased steadily (FIG. 1). In the per-visit LOCF analysis (FAS, ANCOVA) of the PANSS positive subscale scores, treatment with BX20 or with BX30 was statistically significantly superior to that with PBO from Week 6 onwards.

### PANSS Negative

CLINICAL STUDY ONE: Table 8 presents mean PANSS negative subscale scores at Baseline and the mean change from Baseline by visit using LOCF for the ITT population. The bifeprunox 5 mg, 20 mg, and risperidone 6 mg groups showed the greatest improvement over time. Estimates of the treatment effect (bifeprunox—placebo) at Endpoint LOCF were −1.0, −0.3, −1.4 for the Bifeprunox 5 mg, 10 mg, and 20 mg groups respectively. A statistically significantly greater decrease in PANSS negative subscale score was seen at Week 3 (unadjusted p=0.013) and at Endpoint (LOCF) (unadjusted p=0.026) for the bifeprunox 20 mg group versus placebo.

TABLE 8
Change from Baseline in PANSS Negative Subscale Score-Last
Observation Carried Forward at Each Visit Intent-to-Treat Population.

| | | Treatment Group | | | | |
|---|---|---|---|---|---|---|
| | | Bifeprunox | Bifeprunox | Bifeprunox | | Risperidone |
| | Statistic | 5 mg | 10 mg | 20 mg | Placebo | 6 mg |
| Total Number of | N | 110 | 118 | 111 | 114 | 116 |
| Subjects in the | | | | | | |
| ITT Population | | | | | | |
| Baseline | n | 110 | 118 | 111 | 114 | 116 |
| | Mean (S.D.) | 22.4 (4.8) | 23.5 (4.5) | 22.6 (4.8) | 23.1 (4.9) | 22.9 (4.4) |
| | Median | 22 | 23 | 22 | 23 | 23 |
| | Min-Max | 12-38 | 14-37 | 12-37 | 14-42 | 12-38 |
| Change from | | | | | | |
| Baseline | | | | | | |
| Week 1 | n | 110 | 118 | 110 | 114 | 116 |
| | Mean (S.D.) | −1.1 (3.9) | −1.4 (3.4) | −1.6 (3.4) | −1.3 (3.0) | −2.1 (3.5) |
| | Median | −1 | −1 | −1 | −1 | −1 |
| | Min-Max | −15-13 | −16-6 | −18-8 | −11-8 | −16-5 |
| | P-value | 0.781 | 0.875 | 0.372 | | |
| Week 2 | n | 110 | 118 | 110 | 114 | 116 |
| | Mean (S.D.) | −1.8 (4.4) | −1.5 (4.2) | −2.0 (4.2) | −0.9 (4.1) | −3.0 (4.2) |
| | Median | −2 | −1 | −1 | −1 | −3 |
| | Min-Max | −21-13 | −15-9 | −15-11 | −13-15 | −22-7 |
| | P-value | 0.109 | 0.377 | 0.054 | | |
| Week 3 | n | 110 | 118 | 111 | 114 | 116 |
| | Mean (S.D.) | −1.8 (4.7) | −1.7 (5.0) | −2.7 (4.4) | −1.2 (4.6) | −3.5 (4.2) |
| | Median | −2 | −1 | −−2 | −1 | −3 |
| | Min-Max | −21-13 | −16-15 | −18-11 | −13-16 | −22-6 |
| | P-value | 0.290 | 0.526 | 0.013* | | |
| Week 4 | n | 110 | 118 | 111 | 114 | 116 |
| | Mean (S.D.) | −2.0 (4.9) | −1.5 (4.6) | −2.5 (4.6) | −1.3 (4.5) | −3.2 (4.3) |
| | Median | −1 | −1 | −2 | −1 | −3 |
| | Min-Max | −19-13 | −15-10 | −13-14 | −15-16 | −19-6 |
| | P-value | 0.248 | 0.907 | 0.053 | | |
| Endpoint LOCF | n | 110 | 118 | 111 | 114 | 116 |
| | Mean (S.D.) | −2.2 (4.9) | −1.7 (4.7) | −2.6 (5.3) | −1.3 (4.6) | −3.6 (4.6) |
| | Median | −2 | −1 | −2 | −1 | −3 |
| | Min-Max | −20-13 | −16-10 | −16-14 | −16-16 | −21-7 |
| | Trt Effect | −1.0 | 0.3 | −1.4 | | |
| | Trt Effect CI | (−2.2, 0.3) | (−1.5, 1.0) | (−2.6, −0.2) | | |
| | P-value | 0.118 | 0.686 | 0.026* | | |

*Significant at the 0.050 level.
Note:
Pairwise comparisons versus placebo are based on an ANCOVA model with treatment (excluding risperidone) and pooled center as fixed factors and baseline PANSS negative subscale score as a covariate.
Note:
No PANSS scores were recorded at Week 1 or Week 2 for Subject 11259 in the bifeprunox 20 mg group.

For the observed values analysis of change from Baseline in PANSS negative subscale score, decreases in the scores over time indicated improvement in each of the bifeprunox treatment groups. A statistically significant treatment effect of bifeprunox was observed at Week 2 (p=0.032) for the bifeprunox 5 mg group compared to placebo. No other significant differences were noted.

CLINICAL STUDY TWO: Table 9 presents PANSS Negative Symptom subscale scores at Baseline and the change from Baseline by visit using LOCF for the ITT population. The mean change (SD) from Baseline to Endpoint in PANSS Negative Symptom subscale scores was −3.1 (5.6) for the 30 mg bifeprunox group, −2.2 (5.4) for the 40 mg bifeprunox group, −1.8 (5.6) for the placebo group, and −3.8 (5.5) for the risperidone group. The treatment effect values (bifeprunox—placebo) at Endpoint LOCF were: −1.4 for the 30 mg bifeprunox group and −0.9 for the 40 mg bifeprunox group. SEP1 did not yield a significant result, and therefore SEP2 and SEP3 were not evaluated.

The 30 mg bifeprunox treatment group showed a notable difference from placebo (nominal p=0.027) at Endpoint. Notable differences between 30 mg bifeprunox and placebo treatment groups were also noted at Week 2 through Week 4 (p≦0.021). No differences were observed between placebo and the 40 mg bifeprunox dose group at any time point during the study (Week 1 through Week 6).

TABLE 9
Change from Baseline in PANSS Negative Symptom Subscale Score
Last Observation Carried Forward at Each Visit Intent-to-Treat Population.

| | | Treatment Group | | | |
|---|---|---|---|---|---|
| | | Bifeprunox | Bifeprunox | | Risperidone |
| | Statistic | 30 mg | 40 mg | Placebo | 6 mg |
| Total Number of | N | 140 | 141 | 144 | 151 |
| Subjects in the ITT | | | | | |
| Population | | | | | |
| Baseline | n | 140 | 141 | 144 | 151 |
| | Mean (SD) | 23.5 (4.5) | 22.5 (4.4) | 23.4 (5.0) | 22.5 (5.0) |
| | Median | 23 | 22 | 24 | 22 |
| | Min, Max | 12, 33 | 9, 39 | 9, 35 | 11, 37 |
| Change from | | | | | |
| Baseline | | | | | |
| Week 1 | n | 140 | 139 | 143 | 149 |
| | Mean (SD) | −1.6 (3.7) | −1.2 (3.3) | −1.3 (3.4) | −1.5 (3.0) |
| | Median | −1 | −1 | −1 | −2 |
| | Min, Max | −19, 10 | −16, 7 | −15, 11 | −11, 10 |
| | P-value | 0.359 | 0.861 | | |
| Week 2 | n | 140 | 140 | 144 | 151 |
| | Mean (SD) | 2.6 (4.4) | −1.9 (4.0) | −1.5 (4.6) | −2.9 (4.3) |
| | Median | −2 | −2 | −1 | −3 |
| | Min, Max | −19, 9 | −14, 9 | −18, 12 | −18, 9 |
| | P-value | 0.018 | 0.141 | | |
| Week 3 | n | 140 | 140 | 144 | 151 |
| | Mean (SD) | −3.0 (5.0) | −2.2 (4.7) | −1.7 (5.3) | −3.3 (4.9) |
| | Median | −3 | −1 | −2 | −3 |
| | Min, Max | −19, 9 | −22, 9 | −18, 14 | −22, 9 |
| | P-value | 0.021 | 0.138 | | |
| Week 4 | n | 140 | 140 | 144 | 151 |
| | Mean (SD) | −3.1 (5.3) | −2.1 (4.9) | −1.7 (5.5) | −3.8 (5.5) |
| | Median | −2 | −1 | −1 | −4 |
| | Min, Max | −20, 9 | −22, 9 | −19, 14 | −26, 9 |
| | P-value | 0.019 | 0.226 | | |
| Week 6/Endpoint | n | 140 | 141 | 144 | 151 |
| | Mean (SD) | −3.1 (5.6) | −2.2 (5.4) | −1.8 (5.6) | −3.8 (5.5) |
| | Median | −2 | −1 | −1 | −3 |
| | Min, Max | −24, 9 | −24, 9 | −19, 14 | −26, 9 |
| | Trt Effect | −1.4 | −0.9 | | |
| | 95% CI | −2.6, −0.2 | −1.9, 0.4 | | |
| | 97.5% CI | −2.6, 0.2 | −2.1, 0.4 | | |
| | P-value (Raw) | 0.027 | 0.166 | | |

Note:
Treatment comparisons versus placebo were based on an ANCOVA model with treatment (excluding risperidone) and pooled center as fixed factors and Baseline PANSS negative symptom sub-scale score as a covariate.
Note:
p-values presented for Week 1 through Week 4 are for descriptive purposes only.
Trt = Treatment.

CLINICAL STUDY THREE: Table 10 presents PANSS Negative Symptom subscale scores at Baseline and the change from Baseline by visit using LOCF for the ITT population. The mean change (SD) from Baseline to Endpoint in PANSS Negative Symptom subscale score was −3.3 (5.0) for the 20 mg bifeprunox group, −3.1 (5.2) for the 30 mg bifeprunox group, −2.4 (5.1) for the placebo group, and −4.6 (5.2) for the olanzapine group. The treatment effect values (bifeprunox—placebo) at Endpoint LOCF were: −0.9 for the 20 mg bifeprunox group and −0.6 for the 30 mg bifeprunox group. No differences with nominal p-values (≦0.05) were observed between the placebo group and either of the bifeprunox dose groups at Week 6/Endpoint or at any other time point during the study (Week 1 through Week 4).

TABLE 10
Change from Baseline in PANSS Negative Symptom Subscale
Score-Last Observation Carried Forward at Each Visit Intent-to-Treat Population.

| | | Treatment Group | | | |
|---|---|---|---|---|---|
| | | Bifeprunox | Bifeprunox | | Olanzapine |
| | Statistic | 20 mg | 30 mg | Placebo | 15 mg |
| Total Number of | N | 149 | 148 | 145 | 146 |
| Subjects in the ITT | | | | | |
| Population | | | | | |
| Baseline | n | 149 | 148 | 145 | 146 |
| | Mean | 23.0 (4.6) | −23.1 (4.2) | 22.9 (4.0) | 23.5 (4.9) |
| | (SD) | | | | |
| | Median | 23 | 23 | 23 | 24 |
| | Min. Max | 14, 35 | 14, 34 | 15, 35 | 13, 39 |
| Change from | | | | | |
| Baseline | | | | | |
| Week 1 | n | 149 | 146 | 144 | 145 |
| | Mean | −1.5 (3.0) | −1.3 (4.0) | −1.2 (3.2) | −1.8 (3.1) |
| | (SD) | | | | |
| | Median | −1 | −1 | −1 | −1 |
| | Min, Max | −11, 9 | −14, 27 | −17, 8 | −15, 5 |
| | P-value | 0.462 | 0.926 | | |
| Week 2 | n | 149 | 147 | 145 | 146 |
| | Mean | −2.7 (3.6) | −1.9 (4.3) | −2.2 (4.4) | −3.0 (3.8) |
| | (SD) | | | | |
| | Median | −3 | −1 | −2 | −3 |
| | Min, Max | −12, 9 | −14, 23 | −19, 13 | −15, 8 |
| | P-value | 0.281 | 0.412 | | |
| Week 3 | n | 149 | 148 | 145 | 146 |
| | Mean | −3.2 (4.4) | −2.5 (4.8) | −2.4 (4.9) | −4.0 (4.8) |
| | (SD) | | | | |
| | Median | −3 | −2 | −2 | −3 |
| | Min, Max | −15, 9 | −18, 23 | −19, 18 | −20, 8 |
| | P-value | 0.075 | 0.797 | | |
| Week 4 | n | 149 | 148 | 145 | 146 |
| | Mean | −3.3 (4.8) | −2.6 (5.0) | −2.3 (4.9) | −4.0 (4.9) |
| | (SD) | | | | |
| | Median | −3 | −2 | −2 | −4 |
| | Min, Max | −15, 15 | −17, 23 | −19, 18 | −24, 8 |
| | P-value | 0.058 | 0.603 | | |
| Week 6/Endpoint | n | 149 | 148 | 145 | 146 |
| | Mean | −3.3 (5.0) | −3.1 (5.2) | −2.4 (5.1) | −4.6 (5.2) |
| | (SD) | | | | |
| | Median | −3 | −2 | −2 | −4 |
| | Min, Max | −15, 18 | 23, 23 | −19, 18 | −27, 8 |
| | Trt Effect | −0.9 | −0.6 | | |
| | Trt Effect | −2.0, 0.2 | −1.6, 0.5 | | |
| | 95% CI | | | | |
| | P-value | 0.099 | 0.317 | | |

Note:
Treatment comparisons versus placebo were based on an ANCOVA model with treatment (excluding olanzapine) and pooled center as fixed factors and Baseline PANSS negative symptom, subscale score as a covariate.
Note:
Significance at Week 6/Endpoint for multiple comparisons was evaluated according to the Hochberg procedure.
Note:
P-values and CIs presented for Week 1 through Week 4, and Week 6/Endpoint are for descriptive purposes only.
Trt = Treatment.

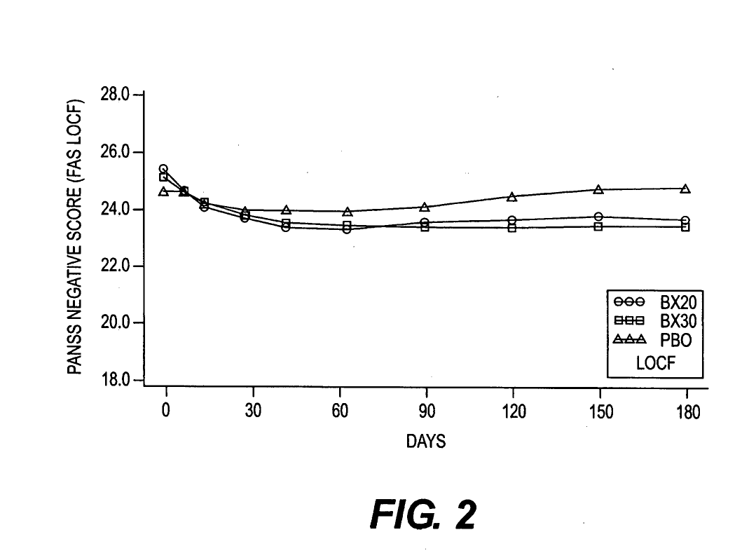

CLINICAL STUDY FOUR: The mean PANSS negative subscale scores (FAS, LOCF) decreased in both BX groups until Week 6, after which the scores stabilized (FIG. 2). In the PBO group, the scores decreased until Week 4, after which the scores tended to increase.

In the per-visit LOCF analysis (FAS, ANCOVA) of the mean PANSS negative subscale scores, treatment with BX20 or with BX20 was statistically significantly superior to that with PBO at all time points, except at Weeks 2 and 4 for BX30.

### PANSS Data for Clinical Study Four

Table 11 shows the mean efficacy variables at baseline, Week 6, and Month 6 (FAS, LOCF). The estimates from the ANCOVA of the changes from baseline in PANSS total scores are provided in Tables 12, 13, and 14. The mean PANSS total scores (FAS, LOCF) decreased over time in both BX groups until Week 9, after which the scores stabilized (Panel 28). In the PBO group, the mean PANSS total scores decreased until Week 2, after which the scores increased steadily.

The LOCF analysis of the PANSS total scores shows that when the last observation was carried forward, the PANSS total scores in the BX groups remained stable. The changes in both BX groups were statistically significantly superior to that in the PBO group from Week 6 onwards. In addition, BX20 was statistically significantly superior to PBO Week 4 onwards. The OC analysis of the PANSS total scores shows that patients who continued in the study improved over time. The changes in both BX groups were statistically significantly superior to that in the PBO group at a number of time points including Week 6, but excluding Month 6.

The differences between the LOCF and POCF analyses indicate that a number of patients who withdrew had a deterioration in their PANSS total score and withdrew at the first visit where a deterioration was seen. This is in line with the study design that required patients to be withdrawn from the study if they were at least minimally worse.

For the other efficacy variables, the trends in the development over time in efficacy scores were similar, except for CDSS, where the baseline levels indicate that the patients included in the study were not depressed.

TABLE 11
Efficacy Scale Scores - Baseline and Adjusted Mean Change from
Baseline (FAS, LOCF).

| | PBO | | BX20 | | BX30 | |
|---|---|---|---|---|---|---|
| | (n = 166) | | (n = 158) | | (n = 172) | |
| PANSS total | | | | | | |
| Baseline | 85.8 | (11.3) | 87.2 | (10.8) | 86.9 | (10.4) |
| Week 6 | 1.1 | (1.2) | −4.0 | (1.2)* | −2.6 | (1.1)* |
| Month 6 | 7.1 | (1.4) | −4.0 | (1.2)* | −0.4 | (1.4)* |
| PANSS positive subscale | | | | | | |
| Baseline | 19.0 | (4.3) | 18.6 | (3.9) | 19.2 | (4.2) |
| Week 6 | 0.5 | (0.4) | −0.9 | (0.3)* | −0.8 | (0.4)* |
| Month 6 | 2.6 | (0.5) | 0.3 | (0.5)* | 0.3 | (0.5)* |
| PANSS negative subscale | | | | | | |
| Baseline | 24.7 | (4.3) | 25.4 | (4.4) | 25.1 | (4.2) |
| Week 6 | −0.7 | (0.3) | −2.0 | (0.3)* | −1.6 | (0.3)* |
| Month 6 | 0.1 | (0.4) | −1.6 | (0.4)* | −1.7 | (0.4)* |
| PANSS general psychopathology | | | | | | |
| Baseline | 42.2 | (6.2) | 43.2 | (6.0) | 42.6 | (5.8) |
| Week 6 | 1.0 | (0.6) | −1.2 | (0.6)* | −0.4 | (0.6) |
| Month 6 | 4.2 | (0.7) | 0.4 | (0.7)* | 0.8 | (0.7)* |
| BPRS total | | | | | | |
| Baseline | 46.5 | (7.1) | 46.8 | (6.7) | 47.0 | (6.7) |
| Week 6 | 0.5 | (0.7) | −2.1 | (0.7)* | −1.3 | (0.7) |
| Month 6 | 4.5 | (0.8) | −0.1 | (0.9)* | 0.5 | (0.8)* |
| BPRS psychosis cluster | | | | | | |
| Baseline | 12.4 | (2.9) | 12.1 | (2.7) | 12.6 | (2.9) |
| Week 6 | 0.1 | (0.2) | −0.8 | (0.2)* | −0.7 | (0.2)* |
| Month 6 | 1.2 | (0.3) | −0.1 | (0.3)* | −0.2 | (0.3)* |
| CDSS total | | | | | | |
| Baseline | 2.2 | (2.9) | 2.2 | (2.7) | 2.5 | (3.5) |
| Week 6 | −0.1 | (0.2) | −0.2 | (0.2) | −0.1 | (0.2) |
| Month 6 | 0.4 | (0.2) | 0.0 | (0.2) | 0.1 | (0.2) |
| CGI-I | | | | | | |
| Week 6^{a} | 4.1 | (0.1) | 3.8 | (0.1)* | 3.7 | (0.1)* |
| Month 6^{a} | 4.6 | (0.1) | 4.0 | (0.1)* | 4.0 | (0.1)* |
| CGI-S | | | | | | |
| Baseline | 4.4 | (0.6) | 4.5 | (0.6) | 4.5 | (0.6) |
| Week 6 | 0.0 | (0.1) | −0.2 | (0.1) | −0.1 | (0.1) |
| Month 6 | 0.2 | (0.1) | −0.1 | (0.1)* | 0.0 | (0.1)* |

Baseline values are mean (SD);
Week 6 and Month 6 values are LS mean (standard error (SE)) from the ANCOVA model with centre and treatment as factors and the baseline value as a covariate.
*statistically significantly different from PBO according to Hochberg's Step-up Method;
p < 0.025 for the individual BX group or p < 0.050 for both BX groups
^{a}Adjusted mean values instead of adjusted mean change from baseline

TABLE 12
Adjusted Mean Change from Baseline in PANSS Total Score (FAS,
LOCF, ANCOVA).

| | | | Least Squares | | | Difference to PBO | | | |
|---|---|---|---|---|---|---|---|---|---|
| Treatment | | | Estimates | | | | 95% CI | 95% CI | |
| Group | Days | n | Mean | SE | Mean | SE | Lower | Upper | p-value |
| PBO | 7 | 166 | −0.59 | 0.49 | | | | | |
| | 14 | 166 | −0.65 | 0.80 | | | | | |
| | 28 | 166 | 0.07 | 1.00 | | | | | |
| | 42 | 166 | 1.06 | 1.17 | | | | | |
| | 63 | 166 | 2.25 | 1.28 | | | | | |
| | 90 | 166 | 3.67 | 1.36 | | | | | |
| | 120 | 166 | 5.30 | 1.38 | | | | | |
| | 150 | 166 | 6.38 | 1.41 | | | | | |
| | 180 | 166 | 7.10 | 1.42 | | | | | |
| BX20 | 7 | 158 | −1.89 | 0.51 | −1.30 | 0.67 | −2.62 | 0.02 | 0.053 |
| | 14 | 158 | −3.03 | 0.82 | −2.38 | 1.09 | −4.51 | −0.24 | 0.029 |
| | 28 | 158 | −.327 | 1.03 | −3.34 | 1.36 | −6.01 | −0.67 | 0.014 |
| | 42 | 158 | −4.01 | 1.20 | −5.07 | 1.59 | −8.17 | −1.95 | 0.002 |
| | 63 | 158 | −3.22 | 1.31 | −5.47 | 1.74 | −8.89 | −2.05 | 0.002 |
| | 90 | 158 | −2.18 | 1.39 | −5.85 | 1.84 | −9.47 | −2.23 | 0.002 |
| | 120 | 158 | −1.20 | 1.41 | −6.50 | 1.87 | −10.18 | −2.82 | 0.001 |
| | 150 | 158 | −0.56 | 1.44 | −7.04 | 1.91 | −10.79 | −3.29 | 0.000 |
| | 180 | 158 | −0.81 | 1.46 | −7.91 | 1.93 | −11.70 | −4.12 | 0.000 |
| BX30 | 7 | 172 | −0.73 | 0.48 | −0.14 | 0.66 | −1.42 | 1.15 | 0.835 |
| | 14 | 172 | −1.45 | 0.77 | −0.79 | 1.06 | −2.88 | 1.29 | 0.455 |
| | 28 | 172 | −2.13 | 0.97 | −2.20 | 1.33 | −4.82 | 0.41 | 0.098 |
| | 42 | 172 | −2.65 | 1.13 | −3.71 | 1.55 | −6.76 | −0.66 | 0.017 |
| | 63 | 172 | −2.44 | 1.24 | −4.68 | 1.70 | −8.03 | −1.34 | 0.006 |
| | 90 | 172 | −1.47 | 1.32 | −5.14 | 1.80 | −8.68 | −1.60 | 0.005 |
| | 120 | 172 | −0.84 | 1.34 | −6.14 | 1.83 | −9.73 | −2.54 | 0.001 |
| | 150 | 172 | −0.31 | 1.36 | −6.80 | 1.87 | −10.46 | −3.13 | 0.000 |
| | 180 | 172 | −0.44 | 1.38 | −7.54 | 1.88 | −11.24 | −3.84 | 0.000 |

TABLE 13
Adjusted Mean Change from Baseline in PANSS Total Score (FAS,
OC, ANCOVA).

| | | | Least Squares | | | Difference to PBO | | | |
|---|---|---|---|---|---|---|---|---|---|
| Treatment | | | Estimates | | | | 95% CI | 95% CI | |
| Group | Days | n | Mean | SE | Mean | SE | Lower | Upper | p-value |
| PBO | 7 | 166 | −0.59 | 0.49 | | | | | |
| | 14 | 149 | −2.38 | 0.77 | | | | | |
| | 28 | 136 | 3.52 | 0.96 | | | | | |
| | 42 | 112 | −6.26 | 1.11 | | | | | |
| | 63 | 92 | −8.33 | 1.35 | | | | | |
| | 90 | 78 | −9.26 | 1.60 | | | | | |
| | 120 | 62 | −10.36 | 1.82 | | | | | |
| | 150 | 52 | −14.39 | 2.02 | | | | | |
| | 180 | 44 | −14.39 | 1.87 | | | | | |
| BX20 | 7 | 158 | −1.89 | 0.51 | −1.30 | 0.67 | −2.62 | 0.02 | 0.053 |
| | 14 | 138 | −4.77 | 0.80 | −2.39 | 1.02 | −4.41 | −0.38 | 0.020 |
| | 28 | 121 | −7.61 | 1.03 | −4.09 | 1.26 | −6.57 | −1.62 | 0.001 |
| | 42 | 104 | −12.40 | 1.17 | −6.14 | 1.49 | −9.07 | −9.07 | 0.000 |
| | 63 | 95 | −12.85 | 1.37 | −4.52 | 1.78 | −8.03 | −1.00 | 0.012 |
| | 90 | 81 | −14.17 | 1.65 | −4.92 | 2.12 | −9.09 | −0.75 | 0.021 |
| | 120 | 69 | −15.81 | 1.84 | −5.45 | 2.42 | −10.23 | −0.66 | 0.026 |
| | 150 | 63 | −16.36 | 2.01 | −1.97 | 2.67 | −7.26 | 3.31 | 0.462 |
| | 180 | 59 | −18.09 | 1.81 | −3.60 | 2.46 | −8.47 | 1.27 | 0.146 |
| BX30 | 7 | 172 | −0.73 | 0.48 | −0.14 | 0.66 | −1.42 | 1.15 | 0.835 |
| | 14 | 137 | −4.43 | 0.80 | −2.05 | 1.02 | −4.06 | −0.04 | 0.046 |
| | 28 | 120 | −7.14 | 1.01 | −3.63 | 1.26 | −6.10 | −1.15 | 0.004 |
| | 42 | 106 | −11.23 | 1.12 | −4.97 | 1.48 | −7.88 | −2.06 | 0.001 |
| | 63 | 98 | −12.45 | 1.33 | −4.12 | 1.77 | −7.60 | −0.64 | 0.021 |
| | 90 | 88 | −12.71 | 1.53 | −3.46 | 2.06 | −7.53 | 0.61 | 0.096 |
| | 120 | 73 | −14.56 | 1.72 | −4.19 | 2.35 | −8.83 | 0.44 | 0.076 |
| | 150 | 64 | −15.93 | 1.93 | −1.54 | 2.61 | −6.70 | 3.62 | 0.556 |
| | 180 | 57 | −18.58 | 1.75 | −4.09 | 2.45 | −8.95 | 0.76 | 0.097 |

TABLE 14
Adjusted Mean Change from Baseline in PANSS Total Score (FAS,
POCF, ANCOVA).

| | | | Least Squares | | | Difference to PBO | | | |
|---|---|---|---|---|---|---|---|---|---|
| Treatment | | | Estimates | | | | 95% CI | 95% CI | |
| Group | Days | n | Mean | SE | Mean | SE | Lower | Upper | p-value |
| PBO | 7 | 154 | −1.47 | 0.39 | | | | | |
| | 14 | 154 | −2.87 | 0.59 | | | | | |
| | 28 | 154 | −4.11 | 0.74 | | | | | |
| | 42 | 154 | −5.07 | 0.84 | | | | | |
| | 63 | 154 | −5.24 | 0.93 | | | | | |
| | 90 | 154 | −9.26 | 1.60 | | | | | |
| | 120 | 154 | −5.09 | 0.96 | | | | | |
| | 150 | 154 | −5.20 | 0.98 | | | | | |
| | 180 | 154 | −5.41 | 1.01 | | | | | |
| BX20 | 7 | 142 | −2.81 | 0.41 | −1.33 | 0.53 | −2.37 | −0.30 | 0.011 |
| | 14 | 142 | −5.48 | 0.62 | −2.61 | 0.80 | −4.19 | −1.04 | 0.001 |
| | 28 | 141 | −7.74 | 0.79 | −3.63 | 1.00 | −5.60 | −1.66 | 0.000 |
| | 42 | 142 | −9.40 | 0.88 | −4.32 | 1.13 | −6.55 | −2.10 | 0.000 |
| | 63 | 142 | −10.03 | 0.95 | −4.83 | 1.22 | −7.23 | −2.43 | 0.000 |
| | 90 | 142 | −10.43 | 0.99 | −5.19 | 1.26 | −7.67 | −2.71 | 0.000 |
| | 120 | 142 | −10.30 | 1.01 | −5.21 | 1.30 | −7.77 | 2.65 | 0.000 |
| | 150 | 142 | −10.40 | 1.03 | −5.20 | 1.33 | −7.82 | −2.59 | 0.000 |
| | 180 | 142 | −10.84 | 1.06 | −5.43 | 1.36 | −8.11 | −2.76 | 0.000 |
| BX30 | 7 | 148 | −2.23 | 0.39 | −0.76 | 0.52 | −1.78 | 0.26 | 0.146 |
| | 14 | 148 | −4.65 | 0.60 | −1.78 | 0.79 | −3.34 | −0.22 | 0.025 |
| | 28 | 148 | −6.85 | 0.75 | −2.74 | 0.99 | −4.69 | −0.79 | 0.006 |
| | 42 | 148 | −8.12 | 0.85 | −3.05 | 1.12 | −5.26 | −0.85 | 0.007 |
| | 63 | 148 | −8.97 | 0.91 | −3.77 | 1.21 | −6.14 | −1.39 | 0.002 |
| | 90 | 148 | −9.25 | 0.94 | −4.00 | 1.25 | −6.46 | −1.55 | 0.001 |
| | 120 | 148 | −9.68 | 0.98 | −4.59 | 1.29 | −7.12 | −2.05 | 0.000 |
| | 150 | 148 | −9.55 | 1.00 | −4.35 | 1.32 | −6.94 | −1.76 | 0.001 |
| | 180 | 148 | −9.92 | 1.02 | −4.52 | 1.35 | −7.16 | −1.87 | 0.001 |

### General Psychopathology Score

CLINICAL STUDY ONE: General psychopathology scores decreased from Baseline to Week 6 for each of the bifeprunox treatment groups as shown in Table 15. The estimate of the treatment effect of bifeprunox using LOCF was −2.2, 0.4, and −2.8 for the bifeprunox 5 mg, 10 mg, and 20 mg groups respectively. The bifeprunox 20 mg group had a statistically significantly greater decrease than placebo at Week 2 (unadjusted p=0.029), Week 3 (unadjusted p=0.032) and Endpoint (LOCF) (unadjusted p=0.016).

TABLE 15
Change from Baseline in PANSS General Psychopathology
Subscale Score-Last Observation Carried Forward at Each Visit Intent-to-Treat Population

| | | Treatment Group | | | | |
|---|---|---|---|---|---|---|
| | | Bifeprunox | Bifeprunox | Bifeprunox | | Risperidone |
| | Statistic | 5 mg | 10 mg | 20 mg | Placebo | 6 mg |
| Total Number of | N | 110 | 118 | 111 | 114 | 116 |
| Subjects in the | | | | | | |
| ITT Population | | | | | | |
| Baseline | N | 110 | 118 | 111 | 114 | 116 |
| | Mean (S.D.) | 44.1 (6.8) | 45.5 (6.5) | 45.4 (6.9) | 44.7 (6.9) | 44.0 (6.5) |
| | Median | 44 | 46 | 46 | 45 | 44 |
| | Min-Max | 31-62 | 32-60 | 31-65 | 31-60 | 31-65 |
| Change from | | | | | | |
| Baseline | | | | | | |
| Week 1 | N | 110 | 118 | 110 | 114 | 116 |
| | Mean (S.D.) | −1.7 (7.6) | −0.9 (6.2) | −3.0 (6.4) | −1.6 (5.5) | −3.4 (5.8) |
| | Median | −2 | 0 | −2 | −1 | −3 |
| | Min-Max | −25-49 | −24-17 | −26-12 | −21-16 | −24-9 |
| | P-value | 0.900 | 0.317 | 0.149 | | |
| Week 2 | n | 110 | 118 | 110 | 114 | 116 |
| | Mean (S.D.) | −2.8 (8.1) | −1.5 (7.7) | −4.4 (7.5) | −2.0 (7.2) | −5.8 (7.1) |
| | Median | −2 | −2 | −5 | −2 | −6 |
| | Min-Max | −29-49 | −23-23 | −27-13 | −25-16 | −30-7 |
| | P-value | 0.427 | 0.511 | 0.029* | | |
| Week 3 | n | 110 | 118 | 111 | 114 | 116 |
| | Mean (S.D.) | −3.3 (8.8) | −1.7 (9.0) | −5.2 (7.9) | −2.5 (8.1) | −6.9 (7.3) |
| | Median | −3 | −1 | −6 | −3 | −6 |
| | Min-Max | −29-49 | −28-23 | −29-16 | −23-22 | −27-7 |
| | P-value | 0.505 | 0.364 | 0.032* | | |
| Week 4 | n | 110 | 118 | 111 | 114 | 116 |
| | Mean (S.D.) | −3.7 (9.1) | −1.4 (8.8) | −5.2 (8.4) | −2.8 (8.6) | −6.3 (7.7) |
| | Median | −4 | −1 | −6 | −3 | −5 |
| | Min-Max | −31-49 | −27-23 | −27-16 | −21-22 | −31-9 |
| | P-value | 0.543 | 0.138 | 0.062 | | |
| Endpoint LOCF | n | 110 | 118 | 111 | 114 | 116 |
| | Mean (S.D.) | −4.4 (9.4) | −2.0 (9.2) | −5.1 (8.8) | −2.0 (8.6) | −6.8 (7.8) |
| | Median | −5 | −1 | −5 | −3 | −5 |
| | Min-Max | −31-49 | −28-23 | −32-16 | −29-22 | −26-9 |
| | Trt Effect | −2.2 | 0.4 | −2.8 | | |
| | Trt Effect CI | (−4.5, 0.1) | (−1.9, 2.6) | (−5.1, −0.5) | | |
| | P-Value (adj) | 0.058 | 0.758 | 0.016* | | |

*Significant at the 0.050 level.
Note:
Pairwise comparisons versus placebo are based on an ANCOVA model with treatment (excluding risperidone) and pooled center as fixed factors and baseline PANSS general psychopathology subscale score
Note:
No PANSS scores were recorded at Week 1 or Week 2 for Subject 11259 in the bifeprunox 20 mg group.

For the observed values analysis of change from Baseline in PANSS general psychopathology subscale score, decreases in the scores over time indicated improvement in each of the bifeprunox treatment groups. A statistically significant treatment effect of bifeprunox versus placebo was observed at Week 2 (p=0.038.) for the bifeprunox 20 mg group versus placebo and at Week 6 (p=0.017) for the bifeprunox 5 mg group. No other significant differences were noted.

CLINICAL STUDY TWO: Mean PANSS general psychopathology subscale scores at Baseline and change from Baseline in PANSS general psychopathology subscale at each post-Baseline visit for the ITT population (LOCF) are presented in Table 16. The mean change (SD) from Baseline to Endpoint in PANSS general psychopathology subscale score was −6.0 (10.2) for the 30 mg bifeprunox group, −3.8 (10.1) for the 40 mg bifeprunox group, −3.5 (9.7) for the placebo group, and −8.6 (9.8) for the risperidone group. The treatment effect values (bifeprunox—placebo) at Endpoint LOCF were: −2.5 for the 30 mg bifeprunox group and −0.7 for the 40 mg bifeprunox group. Notable differences were observed between the 30 mg bifeprunox group and placebo from Week 2 through Endpoint (p≦0.025). There were no notable differences between the 40 mg bifeprunox group and placebo at any timepoint during the study (Week 1 through Endpoint).

TABLE 16
Change from Baseline in General Psychopathology Subscale Score-
Last Observation Carried Forward at Each Visit Intent-to-Treat Population.

| | | Treatment Group | | | |
|---|---|---|---|---|---|
| | | Bifeprunox | Bifeprunox | | Risperidone |
| | Statistic | 30 mg | 40 mg | Placebo | 6 mg |
| Total Number of | N | 140 | 141 | 144 | 151 |
| Subjects in the ITT | | | | | |
| Population | | | | | |
| Baseline | n | 140 | 141 | 144 | 151 |
| | Mean (SD) | 45.9 (6.7) | 44.8 (6.4) | 45.5 (6.8) | 44.9 (6.2) |
| | Median | 46 | 44 | 46 | 45 |
| | Min, Max | 29, 67 | 32, 62 | 28, 59 | 31, 60 |
| | P-value | | | | |
| Change from | | | | | |
| Baseline | | | | | |
| Week 1 | n | 140 | 139 | 143 | 149 |
| | Mean (SD) | −2.7 (6.4) | −2.7 (5.9) | −2.5 (6.4) | −3.9 (5.6) |
| | Median | −2 | −2 | −2 | −4 |
| | Min, Max | −23, 21 | −21, 16 | −31, 18 | −19, 14 |
| | P-value | 0.851 | 0.501 | | |
| Week 2 | n | 140 | 140 | 144 | 151 |
| | Mean (SD) | −4.9 (8.1) | −3.4 (7.9) | −2.7 (8.4) | −6.7 (7.3) |
| | Median | −4 | −3 | −3 | −6 |
| | Min, Max | −23, 30 | −23, 20 | −23, 25 | −21, 13 |
| | P-value | 0.017* | 0.249 | | |
| Week 3 | n | 140 | 140 | 144 | 151 |
| | Mean (SD) | −5.8 (9.2) | −3.8 (8.8) | −.3.3 (9.4) | −7.6 (7.7) |
| | Median | −5 | −3 | −4 | −8 |
| | Min, Max | −30, 30 | −31, 20 | −33, 254 | −24, 13 |
| | P-value | 0.015* | 0.374 | | |
| Week 4 | n | 140 | 140 | 144 | 151 |
| | Mean (SD) | −6.1 (9.7) | −3.4 (9.3) | −3.3 (9.7) | −8.4 (8.5) |
| | Median | −5 | −2 | −4 | −9 |
| | Min, Max | −32, 34 | −31, 20 | −34, 25 | −33, 13 |
| | P-value | 0.007** | 0.638 | | |
| Week 6/Endpoint | n | 140 | 141 | 144 | 151 |
| | Mean (SD) | −6.0 (10.2) | −3.8 (10.1) | −3.5 (9.7) | −8.6 (9.8) |
| | Median | −5 | −3 | −4 | −9 |
| | Min, Max | −37, 30 | −33, 22 | −34, 25 | −35, 26 |
| | Trt Effect | −2.5 | −0.7 | | |
| | 95% CI | 4.7, −0.3 | −2.9, 1.5 | | |
| | P-value | 0.025* | 0.545 | | |

*Notably different from placebo, p ≦ 0.050;
**Notably different from placebo, p ≦ 0.010.
Note:
Treatment comparisons versus placebo were based on an ANCOVA model with treatment (excluding Risperidone) and pooled center as fixed factors and Baseline PANSS general psychopathology sub-scale score as a covariate.
Trt = Treatment.

CLINICAL STUDY THREE: Mean PANSS General Psychopathology subscale scores at Baseline and change from Baseline in PANSS General Psychopathology subscale scores at each post-Baseline visit for the ITT population (LOCF) are presented in Table 17. The mean change (SD) from Baseline to Endpoint in PANSS General Psychopathology subscale score was −6.1 (10.7) for the 20 mg bifeprunox group, −5.8 (10.3) for the 30 mg bifeprunox group, −4.8 (10.0) for the placebo group, and −10.5 (9.4) for the olanzapine group. The treatment effect values (bifeprunox—placebo) at Endpoint LOCF were: −1.5 for the 20 mg bifeprunox group and −0.9 for the 30 mg bifeprunox group. No notable differences were observed between the placebo group and either of the two bifeprunox dose groups at Endpoint or at any other time point during the study (Week 1 through Week 4).

TABLE 17
Change from Baseline in PASS General Psychopathology Subscale
Score Last Observation Carried Forward at Each Visit Intent-to-Treat Population.

| | | Treatment Group | | | |
|---|---|---|---|---|---|
| Total Number of | | Bifeprunox | Bifeprunox | | Olanzapine |
| Subjects in | Statistic | 20 mg | 30 mg | Placebo | 15 mg |
| the ITT Population | N | 149 | 148 | 145 | 146 |
| Baseline | n | 149 | 148 | 145 | 146 |
| | Mean | 46.3 (6.7) | 47.0 (6.7) | 46.5 (7.1) | 47.6 (6.8) |
| | (SD) | | | | |
| | Median | 47 | 48 | 46 | 48 |
| | Min, Max | 31, 61 | 30, 68 | 27, 64 | 31, 65 |
| Change from | | | | | |
| Baseline | | | | | |
| Week 1 | n | 149 | 146 | 144 | 145 |
| | Mean | −2.4 (6.7) | −2.8 (6.3) | −3.1 (5.8) | −4.5 (6.2) |
| | (SD) | | | | |
| | Median | −2 | −3 | −2 | −3 |
| | Min, Max | −24, 28 | −20, 20 | −35, 8 | −34, 5 |
| | P-value | 0.405 | 0.607 | | |
| Week 2 | n | 149 | 147 | 145 | 146 |
| | Mean | −4.6 (8.4) | −3.8 (8.2) | −4.7 (7.6) | −7.2 (7.8) |
| | (SD) | | | | |
| | Median | −5 | −4 | −3 | −6 |
| | Min, Max | −32. 28 | −28, 32 | −28, 11 | −30, 12 |
| | P-value | 0.961 | 0.250 | | |
| Week 3 | n | 149 | 148 | 145 | 146 |
| | Mean | −5.6 (9.3) | −5.0 (9.3) | −5.4 (8.5) | −9.3 (8.3) |
| | (SD) | | | | |
| | Median | −6 | −4 | −5 | −8 |
| | Min, Max | −37, 28 | −30, 32 | −31, 15 | −35, 12 |
| | P-value | 0.786 | 0.545 | | |
| Week 4 | n | 149 | 148 | 145 | 146 |
| | Mean | −5.8 (10.1) | −5-4 (9.5) | −5-2 (9.5) | −9.6 (8.6) |
| | (SD) | | | | |
| | Median | −7 | −5 | −4 | −9 |
| | Min, Max | −39, 28 | −30, 32 | −34, 15 | −38, 12 |
| | P-value | 0.471 | 0.981 | | |
| Week 6/Endpoint | n | 149 | 148 | 145 | 146 |
| | Mean | −6.1 (10.7) | −5.8 (10.3) | −4.8 (10.0) | −10.5 (9.4) |
| | (SD) | | | | |
| | Median | −7 | −6 | −3 | −9 |
| | Min, Max | −39, 28 | −30, 32 | −33, 21 | −39, 12 |
| | Trt Effect | −1.5 | −0.9 | | |
| | Trt Effect | −3.7, 0.7 | −3.1, 1.4 | | |
| | 95%, CI | | | | |
| | P-value | 0.188 | 0.446 | | |

Note:
Treatment comparisons versus placebo were based on an ANCOVA model with treatment (excluding olanzapine) and pooled center as fixed factors and Baseline PANSS general psychopathology subscale score as a covariate.
Trt = Treatment.

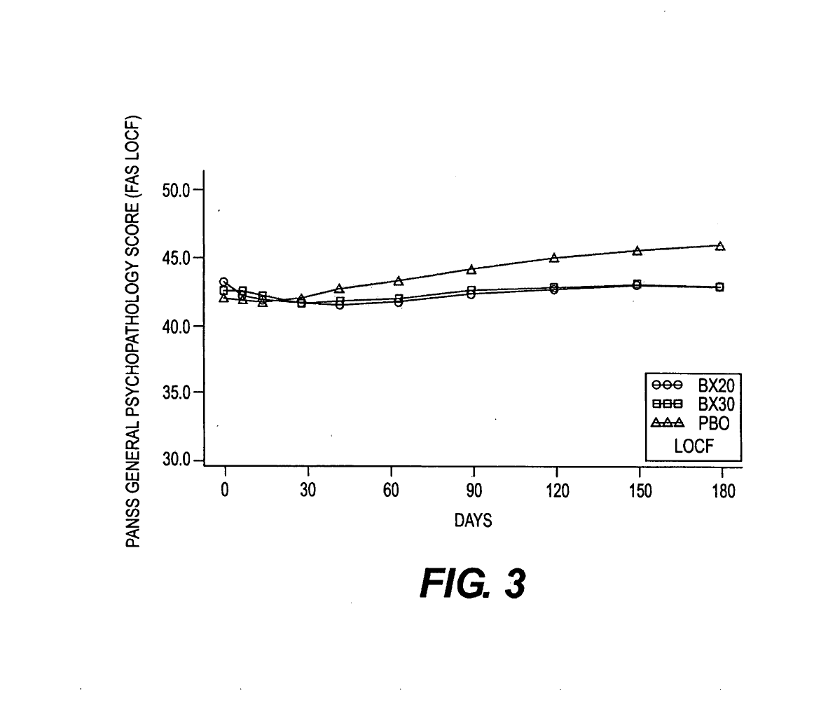

CLINICAL STUDY FOUR: The mean PANSS general psychopathology subscale scores generally followed the same pattern as the PANSS total scores. The mean PANSS general psychopathology subscale scores (FAS, LOCF) decreased in both BX groups until Week 6, after which the scores stabilized (FIG. 3). In the PBO group, the mean PANSS general psychopathology subscale scores decreased until Week 2, after which the scores increased steadily.

In the per-visit LOCF analysis (FAS, ANCOVA) of the PANSS general psychopathology subscale scores, both BX20 and BX30 were statistically significantly superior to PBO from Week 9 onwards. In addition, BX20 was statistically significantly superior to PBO at Week 6.

### BPRS Total Score

CLINICAL STUDY ONE: As shown in Table 18, the bifeprunox 20 mg group showed greater change in BPRS total score than the placebo group. Statistically significant treatment group differences were seen for bifeprunox 20 mg versus placebo at every time point beginning at Week 2 (unadjusted p=0.042); Week 3 (unadjusted p=0.020); Week 4 (unadjusted p=0.024); Endpoint (LOCF) (unadjusted p=0.012).

TABLE 18
Change from Baseline in BPRS Total Score-Last Observation
Carried Forward at Each Visit Intent-to-Treat Population.

| | | Treatment Group | | | | |
|---|---|---|---|---|---|---|
| | | Bifeprunox | Bifeprunox | Bifeprunox | | Risperidone |
| | Statistic | 5 mg | 10 mg | 20 mg | Placebo | 6 mg |
| Total Number of | N | 110 | 118 | 111 | 114 | 116 |
| Subjects in the | | | | | | |
| ITT Population | | | | | | |
| Baseline | n | 110 | 118 | 111 | 114 | 116 |
| | Mean (S.D.) | 53.3 (6.3) | 54.8 (6.9) | 55.1 (7.1) | 54.2 (7.1) | 52.9 (7.2) |
| | Median | 53 | 54 | 54 | 54 | 52 |
| | Min-Max | 41-67 | 39-77 | 41-78 | 40-74 | 36-80 |
| Change from | | | | | | |
| Baseline | | | | | | |
| Week 1 | n | 110 | 118 | 110 | 114 | 116 |
| | Mean (S.D.) | −2.4 (7.5) | −2.0 (7.2) | −4.3 (7.3) | −2.4 (6.3) | −4.6 (6.5) |
| | Median | −3 | −1 | −3 | −2 | −3 |
| | Min-Max | −23-44 | −27-16 | −31-12 | −22-17 | −25-10 |
| | P-value | 0.999 | 0.540 | 0.086 | | |
| Week 2 | n | 110 | 118 | 110 | 114 | 116 |
| | Mean (S.D.) | −4.2 (8.6) | −3.1 (9.3) | −6.1 (8.8) | −3.6 (8.5) | −7.6 (8.4) |
| | Median | −4 | −3 | −7 | −3 | −7 |
| | Min-Max | −29-44 | −29-24 | −36-16 | −32-20 | −36-10 |
| | P-value | 0.649 | 0.493 | 0.042* | | |
| Week 3 | n | 110 | 118 | 111 | 114 | 116 |
| | Mean (S.D.) | −5.2 (9.7) | −3.3 (10.9) | −7.5 (9.1) | −4.3 (9.7) | −9.1 (8.4) |
| | Median | −5 | −2 | −8 | −4 | −9 |
| | Min-Max | −32-44 | −34-27 | −37-19 | −33-22 | −32-10 |
| | P-value | 0.502 | 0.367 | 0.020* | | |
| Week 4 | n | 110 | 118 | 111 | 114 | 116 |
| | Mean (S.D.) | −5.6 (10.2) | −3.1 (10.7) | −7.7 (9.7) | −4.5 (10.2) | −8.6 (9.0) |
| | Median | −5 | −3 | −8 | −4 | −7 |
| | Min-Max | −33-44 | −34-27 | −35-19 | −25-22 | −35-10 |
| | P-value | 0.456 | 0.207 | 0.024* | | |
| Endpoint LOCF | n | 110 | 118 | 111 | 114 | 116 |
| | Mean (S.D.) | −6.2 (10.5) | −3.4 (11.5) | −7.6 (10.1) | −3.8 (10.03) | −9.2 (8.9) |
| | Median | −5 | −2 | −8 | −5 | −9 |
| | Min-Max | −34-44 | −34-27 | −40-19 | −30-22 | −31-10 |
| | Trt Effect | −2.4 | −0.6 | −3.5 | | |
| | Trt Effect CI | (−5.2, 0.3) | (−2.1, 3.3) | (−6.2, −0.8) | | |
| | P-value | 0.081 | 0.666 | 0.012* | | |

*Significant at the 0.050 level.
Note:
Pairwise comparisons versus placebo are based on an ANCOVA model with treatment (excluding risperidone) and pooled center as fixed factors and baseline BPRS total score as a covariate.
Note:
No PANSS scores were recorded at Week 1 or Week 2 for Subject 11259 in the bifeprunox 20 mg group.

Similar trends observed in the LOCF analysis were noted in the change from Baseline in BPRS total score analysis using observed values. The comparison of bifeprunox treatment effect to placebo was statistically significant at Week 6 (p=0.024) for the 5 mg group only. No other significant comparisons were noted.

CLINICAL STUDY TWO: Table 19 presents mean BPRS total scores (derived from the PANSS) at Baseline and change from Baseline in BPRS total scores at each post-Baseline visit for the ITT population (LOCF). The mean change (SD) from Baseline to Endpoint in BPRS total score was −8.1 (12.3) for the 30 mg bifeprunox group, −6.5 (11.7) for the 40 mg bifeprunox group, −4.9 (11.5) for the placebo group, and −12.2 (11.7) for the risperidone group. The treatment effect values (bifeprunox—placebo) at Endpoint LOCF were: −3.2 for the 30 mg bifeprunox group and −1.9 for the 40 mg bifeprunox group. Notable differences were observed between the 30 mg bifeprunox group and placebo from Week 2 through Endpoint (p≦0.019). No notable differences were seen between the 40 mg bifeprunox group and placebo at any time point during the study (Week 1 through Endpoint).

TABLE 19
Change from Baseline in BPRS Total Score-Last Observation
Carried Forward at Each Visit Intent-to-Treat Population.

| | | Treatment Group | | | |
|---|---|---|---|---|---|
| | | Bifeprunox | Bifeprunox | | Risperidone |
| | Statistic | 30 mg | 40 mg | Placebo | 6 mg |
| Total Number of | N | 140 | 141 | 144 | 151 |
| Subjects in | | | | | |
| the ITT Population | | | | | |
| Baseline | n | 140 | 141 | 144 | 151 |
| | Mean (SD) | 54.9 (7.2) | 54.1 (6.9) | 54.4 (7.2) | 54.4 (7.1) |
| | Median | 54 | 54 | 53 | 54 |
| | Min, Max | 41, 73 | 39, 69 | 38, 71 | 38, 73 |
| Change from | | | | | |
| Baseline | | | | | |
| Week 1 | n | 140 | 139 | 143 | 149 |
| | Mean (SD) | −3.8 (7.0) | −4.3 (7.1) | −3.5 (7.1) | −5.5 (6.3) |
| | Median | −3 | −4 | −4 | −5 |
| | Min, Max | −26, 18 | −27, 20 | −33, 22 | −23, 12 |
| | P-value | 0.733 | 0.249 | | |
| Week 2 | n | 140 | 140 | 144 | 151 |
| | Mean (SD) | −7.0 (9.5) | −5.5 (9.3) | −3.9 (9.7) | −9.1 (8.5) |
| | Median | −6 | −6 | −4 | −9 |
| | Min, Max | −28, 21 | −36, 18 | −30, 32 | −25, 14 |
| | P-value | 0.004* | 0.074 | | |
| Week 3 | n | 140 | 140 | 144 | 151 |
| | Mean (SD) | −7.93 (10.7) | −6.3 (10.3) | −4.6 (10.9) | −10.7 (9.4) |
| | Median | −6 | −6 | −5 | −11 |
| | Min, Max | −36, 21 | −37, 19 | −41, 32 | −36, 14 |
| | P-value | 0.006** | 0.096 | | |
| Week 4 | n | 140 | 140 | 144 | 151 |
| | Mean (SD) | −8.2 (11.5) | −5.7 (10.9) | −4.6 (11.4) | −11.9 (10.0) |
| | Median | −7 | −4 | −5 | −13 |
| | Min, Max | −37.21 | −37, 19 | −49, 32 | −35, 14 |
| | P-value | 0.005** | 0.267 | | |
| Week 6/Endpoint | n | 140 | 141 | 144 | 151 |
| | Mean (SD) | −8.1 (12.3) | −6.5 (11.7) | −4.9 (11.5) | −12.2 (11.7) |
| | Median | −7 | −5 | −5 | −13 |
| | Min, Max | −41, 21 | −39, 22 | −42, 32 | −39, 31 |
| | Trt Effect | −3.2 | −1.9 | | |
| | 95% CI | −5.8, −0.5 | −4.5, 0.8 | | |
| | P-value | 0.019* | 0.163 | | |

*Notably different from placebo, p ≦ 0.050;
***Notably different from placebo, p ≦ 0.010.
Note:
Treatment comparisons versus placebo were based on an ANCOVA model with treatment (excluding risperidone) and center as fixed factors and Baseline BPRS total score as a covariate.
Trt = Treatment.

CLINICAL STUDY THREE: Table 20 presents mean BPRS total scores (derived from the PANSS) at Baseline and change from Baseline in BPRS total scores at each post-Baseline visit for the ITT population (LOCF). The mean change (SD) from Baseline to Endpoint in BPRS total score was −8.5 (11.9) for the 20 mg bifeprunox group, −7.9 (12.0) for the mg bifeprunox group, −6.7 (11.7) for the placebo group, and −13.2 (10.7) for the olanzapine group. The treatment effect values (bifeprunox—placebo) at Endpoint LOCF were: −2.1 for the mg bifeprunox group and −1.1 for the 30 mg bifeprunox group. No notable differences were observed between the placebo group and either of the two bifeprunox dose groups at Endpoint or at any other time point during the study (Week 1 through Week 4).

TABLE 20
Change from Baseline in BPRS Total Score-Last Observation
Carried Forward at Each Visit Intent-to-Treat Population.

| | | Treatment Group | | | |
|---|---|---|---|---|---|
| | | Bifeprunox | Bifeprunox | | Olanzapine |
| | Statistic | 20 mg | 30 mg | Placebo | 15 mg |
| Total Number of Subjects in | N | 149 | 148 | 145 | 146 |
| the ITT Population | | | | | |
| Baseline | n | 149 | 148 | 145 | 146 |
| | Mean (SD) | 54.4 (6.8) | 55.4 (6.7) | 55.0 (7.1) | 55.6 (6.9) |
| | Median | 55 | 56 | 55 | 56 |
| | Min, Max | 37, 70 | 37, 76 | 36, 74 | 40, 73 |
| Change from Baseline | | | | | |
| Week 1 | n | 149 | 146 | 144 | 145 |
| | Mean (SD) | −3.2 (7.3) | −3.6 (7.3) | −4.1 (7.0) | −5.6 (6.7) |
| | Median | −3 | −4 | −3 | −4 |
| | Min, Max | −29, 28 | 28, 26 | −42, 12 | −37, 8 |
| | P-value | 0.350 | 0.477 | | |
| Week 2 | n | 149 | 147 | 145 | 146 |
| | Mean (SD) | −6.0 (9.1) | −5.2 (9.3) | −6.5 (9.2) | −8.9 (8.2) |
| | Median | −7 | −6 | −5 | −8 |
| | Min, Max | −37, 28 | −33, 35 | −33, 16 | −37, 10 |
| | P-value | 0.751 | 0.219 | | |
| Week 3 | n | 149 | 148 | 145 | 146 |
| | Mean (SD) | −7.5 (10.3) | −6.7 (10.7) | −7.5 (10.4) | −11.4 (9.3) |
| | Median | −8 | −7 | −7 | −11 |
| | Min, Max | −41, 28 | −35, 35 | −38, 22 | −39, 10 |
| | P-value | 0.867 | 0.446 | | |
| Week 4 | n | 149 | 148 | 145 | 146 |
| | Mean (SD) | −8.1 (11.3) | −7.3 (10.9) | −7.1 (11.4) | −12.0 (9.8) |
| | Median | −9 | −8 | −6 | −11 |
| | Min, Max | −42, 28 | −36, 35 | −35, 22 | −38, 10 |
| | P-value | 0.316 | 0.957 | | |
| Week 6/Endpoint | n | 149 | 148 | 145 | 146 |
| | Mean (SD) | −8.5 (11.9) | −7.9 (12.0) | −6.7 (11.7) | −13.2 (10.7) |
| | Median | −8 | −9 | −5 | −12 |
| | Min, Max | −42, 30 | −43, 35 | −40, 22 | −42, 10 |
| | Trt Effect | −2.1 | −1.1 | | |
| | Trt Effect | −4.7, 0.5 | −3.7, 1.5 | | |
| | 95% CI | | | | |
| | P-value | 0.115 | 0.411 | | |

Note:
Treatment comparisons versus placebo were based on an ANCOVA model with treatment (excluding olanzapine) and pooled center as fixed factors and Baseline BPRS total score as a covariate.
Trt = Treatment.

### BPRS Psychosis Cluster Score

CLINICAL STUDY ONE: Table 21 presents mean BPRS psychosis cluster score at Baseline and the mean change from Baseline by visit using LOCF for the ITT population. The treatment effect values (bifeprunox—placebo) at Endpoint LOCF were −0.9, 0.4, −1.1 for the Bifeprunox 5 mg, 10 mg, and 20 mg groups respectively. A statistically significantly greater decrease in the difference between bifeprunox and placebo mean change from Baseline (unadjusted p=0.044) at Endpoint (LOCF) was seen in BPRS psychosis cluster score for the bifeprunox 20 mg group versus placebo.

TABLE 21
Change from Baseline in BPRS Psychosis Cluster Score-Last
Observation Carried Forward at Each Visit Intent-to-Treat Population.

| | | Treatment Group | | | | |
|---|---|---|---|---|---|---|
| | | Bifeprunox | Bifeprunox | Bifeprunox | | Risperidone |
| | Statistic | 5 mg | 10 mg | 20 mg | Placebo | 6 mg |
| Total Number | N | 110 | 118 | 111 | 114 | 116 |
| of | | | | | | |
| Subjects in the | | | | | | |
| ITT | | | | | | |
| Population | | | | | | |
| Baseline | n | 110 | 118 | 111 | 114 | 116 |
| | Mean | 18.0 (2.6) | 18.4 (2.)7 | 18.4 (2.5) | 18.4 (2.9) | 18.0 (2.6) |
| | (S.D.) | | | | | |
| | Median | 18 | 18 | 18 | 18 | 17 |
| | Min-Max | 13-26 | 13-27 | 13-26 | 12-27 | 12-26 |
| Change from | | | | | | |
| Baseline | | | | | | |
| Week 1 | n | 110 | 118 | 110 | 114 | 116 |
| | Mean | −1.0 (2.6) | −0.7 (3.0) | −1.5 (2.6) | −0.9 (2.6) | −2.2 (2.7) |
| | (S.D.) | | | | | |
| | Median | −1 | −1 | −1 | −1 | −2 |
| | Min-Max | −9-13 | −10-8 | −9-4 | −11-7 | −12-4 |
| | P-value | 0.897 | 0.519 | 0.110 | | |
| Week 2 | n | 110 | 118 | 110 | 114 | 116 |
| | Mean S.D. | −1.8 (3.3) | −1.1 (3.7) | | −1.6(3.4) | −3.6 (3.2) |
| | Median | −2 | −1 | −2 | −2 | −3 |
| | Min-Max | −10-13 | −11-8 | −12-9 | −12-7 | −13-4 |
| | P-value | 0.756 | 0.204 | 0.149 | | |
| Week 3 | n | 110 | 118 | 111 | 114 | 116 |
| | Mean | −2.2 (3.6) | −1.1 (4.2) | −2.7 (3.5) | −1.7 (3.9) | −4.1 (3.4) |
| | (S.D.) | | | | | |
| | Median | −2 | −1 | −3 | −2 | −4 |
| | Min-Max | −14-13 | −12-12 | −11-9 | −12-9 | −13-3 |
| | P-value | 0.448 | 0.151 | 0.058 | | |
| Week 4 | n | 110 | 118 | 111 | 114 | 116 |
| | Mean | −24 (3.8) | −1.1 (4.3) | −2.8 (3.7) | −1.8 (3.9) | −3.9 (3.3) |
| | (S.D.) | | | | | |
| | Median | −3 | −1 | −3 | −2 | −4 |
| | Min-Max | −14-13 | −11-12 | −10-9 | −11-8 | −13-2 |
| | P-value | 0.278 | 0.152 | 0.060 | | |
| Endpoint LOCF | n | 110 | 118 | 111 | 114 | 116 |
| | Mean | 2.5 (3.8) | −1.2 (4.6) | −2.7 (3.9) | −1.6 (4.1) | −4.1 (3.5) |
| | (S.D.) | | | | | |
| | Median | −3 | 0 | −3 | −2 | −4 |
| | Min-Max | −14-13 | −13-11 | −11-9 | −11-9 | −13-2 |
| | Trt Effect | −0.9 | 0.4 | −1.1 | | |
| | Trt Effect | (−2.0, 0.2) | (−0.6, 1.5) | (−2.2, −0.0) | | |
| | CI | | | | | |
| | P-value | 0.097 | 0.419 | 0.044* | | |

*Significant at the 0.050 level.
Note:
Pairwise comparisons versus placebo are based on an ANCOVA model with treatment (excluding risperidone) and pooled center as fixed factors and baseline BPRS psychosis cluster score as a covariate.
Note:
No PANSS scores were recorded at Week 1 or Week 2 for Subject 11259 in the bifeprunox 20 mg group.

For the observed values analysis of change from Baseline in BPRS psychosis cluster score, larger decreases in the scores compared to placebo were seen for each of the three bifeprunox treatment groups beginning at Week 4 No statistically significant differences were noted.

CLINICAL STUDY TWO: Table 22 presents mean BPRS psychosis cluster score at Baseline and the mean change from Baseline by visit using LOCF for the ITT population. The mean change (SD) from Baseline to Endpoint in BPRS psychosis cluster score was −3.2 (4.2) for the 30 mg bifeprunox group, −2.6 (4.4) for the 40 mg bifeprunox group, −1.8 (3.5) for the placebo group, and −4.9 (4.0) for the risperidone group The treatment effect values (bifeprunox—placebo) at Endpoint LOCF were: −1.4 for the 30 mg bifeprunox group and −1.0 for the 40 mg bifeprunox group. Notable differences were observed between the 30 mg bifeprunox group and placebo from Week 2 through Endpoint (p≦0.002). Notable differences were seen between the 40 mg bifeprunox group and placebo at Endpoint (p=0.031) and at Week 2 and Week 3 (p≦0.036).

TABLE 22
Change from Baseline in BPRS Psychosis Cluster Score-Last
Observation Carried Forward at Each Visit Intent-to-Treat Population.

| | | Treatment Group | | | |
|---|---|---|---|---|---|
| | | Bifeprunox | Bifeprunox | | Risperidone |
| | Statistic | 30 mg | 40 mg | Placebo | 6 mg |
| Total Number of | N | 140 | 141 | 144 | 151 |
| Subjects in the ITT | | | | | |
| Population | | | | | |
| Baseline | n | 140 | 141 | 144 | 151 |
| | Mean (SD) | 16.4 (2.5) | 16.1 (2.5) | 16.3 (2.4) | 16.5 (2.4) |
| | Median | 16 | 16 | 16 | 16 |
| | Min, Max | 12, 24 | 12, 24 | 10, 24 | 10, 23 |
| Change from | n | 140 | 139 | 143 | 149 |
| Baseline: | | | | | |
| Week 1 | Mean (SD) | −1.7 (2.3) | −1.7 (2.6) | −1.2 (2.3) | −2.3 (2.4) |
| | Median | −1 | −1 | −1 | −2 |
| | Min, Max | −9, 3 | −10, 6 | −13, 7 | −8, 4 |
| | P-value | 0.105 | 0.089 | | |
| Week 2 | n | 140 | 140 | 144 | 151 |
| | Mean (SD) | −2.8, (3.4) | −2.2 (3.5) | −1.5 (3.1) | −3.6 (3.0) |
| | Median | −2 | −2 | −1 | −4 |
| | Min, Max | −11, 6 | −12, 5 | −11, 7 | −11, 4 |
| | P-value | <0.001** | 0.031* | | |
| Week 3 | n | 140 | 140 | 144 | 151 |
| | Mean (SD) | −3.1 (3.8) | −2.5 (4.0) | −1.7 (3.4) | −4.3 (3.4) |
| | Median | −2 | −2 | −2 | −4 |
| | Min, Max | −13, 6 | −14, 7 | −13, 7 | −12, 4 |
| | P-value | 0.002** | 0.036* | | |
| Week 4 | n | 140 | 140 | 144 | 151 |
| | Mean (SD) | −3.1 (3.8) | −2.5 (4.2) | −1.8 (3.5) | −4.7 (3.6) |
| | Median | −3 | −1 | −2 | −5 |
| | Min, Max | −13, 6 | −14, 7 | −13, 7 | −13, 4 |
| | P-value | 0.002** | 0.071 | | |
| Week 6/Endpoint | n | 140 | 140 | 144 | 151 |
| | Mean (SD) | −3.2 (4.2) | −2.6 (4.4) | −1.8 (3.5) | −4.9 (4.0) |
| | Median | −3 | −2 | −1 | −5 |
| | Min, Max | −13, 6 | −15, 7 | −13, 7 | −15, 7 |
| | Trt Effect | −1.4 | −1.0 | | |
| | Trt Effect | −2.3, −0.5 | −1.9, −0.1 | | |
| | 95% CI | | | | |
| | P-value | 0.002** | 0.031* | | |

*Notably different from placebo, p ≦ 0.050.
**Notably different from placebo, p ≦ 0.010.
Note:
Treatment comparisons versus placebo were based on an ANCOVA model with treatment (excluding risperidone) and pooled center as fixed factors and Baseline BPRS psychosis cluster score as a covariate
Trt = Treatment.

CLINICAL STUDY THREE: Table 23 presents mean BPRS psychosis cluster score at Baseline and the mean change from Baseline by visit using LOCF for the ITT population. The mean change (SD) from Baseline to

Endpoint in BPRS psychosis cluster score was −3.1 (3-9) for the 20 mg bifeprunox group, −3.2 (4.4) for the 30 mg bifeprunox group, −2.6 (4.0) for the placebo group, and −4.9 (4.0) for the olanzapine group. The treatment effect values (bifeprunox—placebo) at Endpoint LOCF were: −0.6 for the 20 mg bifeprunox group and −0.5 for the 30 mg bifeprunox group. No notable differences were observed between the placebo group and either of the two bifeprunox dose groups at Endpoint or at any other time point during the study (Week 1 through Week 4).

TABLE 23
Change from Baseline in BPRS Psychosis Cluster Score-Last
Observation Carried Forward at Each Visit Intent-to-Treat Population.

| | | Treatment Group | | | |
|---|---|---|---|---|---|
| | | Bifeprunox | Bifeprunox | | Olanzapine |
| | Statistic | 20 mg | 30 mg | Placebo | 15 mg |
| Total Number of Subjects in | N | 149 | 148 | 145 | 146 |
| the ITT Population | | | | | |
| Baseline | n | 149 | 148 | 145 | 146 |
| | Mean (SD) | 16.1 (2.0) | 16.8 (2.3) | 16.6 (2.4) | 16.7 (2.3) |
| | Median | 16 | 16 | 16 | 17 |
| | Min, Max | 11, 22 | 12, 23 | 12, 23 | 11, 23 |
| Change from Baseline | | | | | |
| Week 1 | n | 149 | 146 | 144 | 145 |
| | Mean (SD) | −1.4 (2.3) | −1.7 (2.5) | −1.6 (2.5) | −1.9 (2.3) |
| | Median | −1 | −2 | −1 | −2 |
| | Min, Max | −10, 4 | −8, 6 | −15, 5 | −13, 3 |
| | P-value | 0.871 | 0.824 | | |
| Week 2 | n | 149 | 147 | 145 | 146 |
| | Mean (SD) | −2.1 (2.9) | −2.3 (3.3) | −2.4 (3.1) | −3.1 (2.6) |
| | Median | −2 | −2 | −2 | −3 |
| | Min, Max | −12, 4 | −11, 8 | −11, 6 | −10, 3 |
| | P-value | 0.618 | 0.580 | | |
| Week 3 | n | 149 | 148 | 145 | 146 |
| | Mean (SD) | −2.6 (3.4) | −2.7 (3.8) | −2.9 (3.6) | −4.0 (3.2) |
| | Median | −3 | −3 | −3 | −4 |
| | Min, Max | −14, 5 | −12, 8 | −14, 7 | −11, 2 |
| | P-value | 0.697 | 0.460 | | |
| Week 4 | n | 149 | 148 | 145 | 146 |
| | Mean (SD) | −3-0 (3.7) | −3.0 (4.0) | −2.8 (3.9) | −4.4 (3.5) |
| | Median | −3 | −3 | −3 | −5 |
| | Min, Max | −14, 6 | −13, 8 | −16, 7 | −13, 3 |
| | P-value | 0.550 | 0.873 | | |
| Week 6/Endpoint | n | 149 | 148 | 145 | 146 |
| | Mean (SD) | −3.1 (3.9) | −3.2 (4.4) | −2.6 (4.0) | −4.9 (4.0) |
| | Median | −3 | −3 | −2 | −5 |
| | Min, Max | −14, 9 | −16, 8 | −16, 7 | −15, 3 |
| | Trt Effect | −0.6 | −0.5 | | |
| | Trt Effect | −1.5, 0.3 | −1.4, 0.4 | | |
| | 95% CI | | | | |
| | P-value | 0.165 | 0.245 | | |

Note.
Treatment comparisons versus placebo were based on an ANCOVA model with treatment (excluding olanzapine) and pooled center as fixed factors and Baseline psychosis cluster score as a covariate.
Trt = Treatment.

### CGI-S

CLINICAL STUDY ONE: Table 24 presents mean CGI Severity of Illness scores at Baseline and the mean change from Baseline by visit using LOOF for the ITT population. The treatment effect values (bifeprunox—placebo) at Endpoint LOCF were −0.31, 0.12, −0.19 for the Bifeprunox 5 mg, 10 mg, and 20 mg groups respectively. Pairwise treatment group comparisons showed statistically significantly greater decreases in the bifeprunox 20 mg versus placebo at Week 2 (p=0.008), Week 3 (p=0.020) and Week 4 (p=0.032), but not at Endpoint (LOCF). The treatment group comparison for bifeprunox 5 mg vs placebo was significant at Week 3 (p=0.049), Week 4 (p=0.033), and Endpoint (LOCF) (p=0.013).

TABLE 24
Change from Baseline in CGI Severity of Illness Score-Last
Observation Carried Forward at Each Visit Intent-to-Treat Population.

| | | Treatment Group | | | | |
|---|---|---|---|---|---|---|
| | | Bifeprunox | Bifeprunox | Bifeprunox | | Risperidone |
| | Statistic | 5 mg | 10 mg | 20 mg | Placebo | 6 mg |
| Total | N | 110 | 118 | 111 | 114 | 116 |
| Number of | | | | | | |
| Subjects | | | | | | |
| in the ITT | | | | | | |
| population | | | | | | |
| Baseline | n | 110 | 118 | 111 | 114 | 116 |
| | Mean | 4.57 (0.63) | 4.64 (0.69) | 4.68 (0.66) | 4.54 (0.64) | 4.60 (0.68) |
| | (S.D.) | | | | | |
| | Median | 5.0 | 5.0 | 5.0 | 4.0 | 4.5 |
| | Min-Max | 4.0-7.0 | 4.0-6.0 | 4.0-6.0 | 4.0-6.0 | 4.0-7.0 |
| Change | | | | | | |
| from | | | | | | |
| Baseline | | | | | | |
| Week 1 | n | 110 | 117 | 111 | 114 | 115 |
| | Mean | −0.18 (0.68) | 0.13 (0.75) | −0.31 (0.74) | −0.16 (0.60) | −0.37 (0.68) |
| | (S.D.) | | | | | |
| | Median | 0.0 | 0.0 | 0.0 | 0.0 | 0.0 |
| | Min-Max | −3.0-1.0 | −3.0-2.0 | −4.0-1.0 | −2.0-1.0 | −3.0-2.0 |
| | P-value | 0.754 | 0.542 | 0.172 | | |
| Week 2 | N | 110 | 117 | 111 | 114 | 115 |
| | Mean | −0.37 (0.83) | −2.3 (0.92) | −0.50 (0.85) | −0.18 (0.72) | −0.60 (0.83) |
| | (S.D.) | | | | | |
| | Median | 0.0 | 0.0 | 0.0 | 0.0 | 0.0 |
| | Min-Max | −3.0-1.0 | −3.0-2.0 | −4.0-1.0 | −2.0-1.0 | −3.0-2.0 |
| | P-value | 0.060 | 0.856 | 0.008* | | |
| Week 3 | n | 110 | 117 | 111 | 114 | 115 |
| | Mean | −0.47 (0.90) | −0.22 (1.01) | −0.57 (0.90) | −0.24 (0.98) | −0.70 (0.81) |
| | (S.D.) | | | | | |
| | Median | 0.0 | 0.0 | 0.0 | 0.0 | 0.0 |
| | Min-Max | −3.0-2.0 | −4.0-2.0 | −3.0-1.0 | −4.0-2.0 | −2.0-1.0 |
| | P-value | 0.049* | 0.623 | 0.020* | | |
| Week 4 | n | 110 | 117 | 111 | 114 | 115 |
| | Mean | −0.52 (1.01) | −0.16 (0.99) | −0.57 (0.89) | −0.25 (0.84) | −0.69 (0.85) |
| | (S.D.) | | | | | |
| | Median | 0.0 | 0.0 | 0.0 | 0.0 | 0.0 |
| | Min-Max | −3.0-2.0 | −4.0-2.0 | −3.0-1.0 | −3.0-2.0 | −3.0-1.0 |
| | P-value | 0.03* | 0.276 | 0.02* | | |
| Endpoint | N | 110 | 117 | 111 | 114 | 115 |
| LOCF | Mean | −0.58 (1.10) | −0.18 (1.04) | −0.52 (0.96) | −0.25 (0.97) | −0.76 (0.93) |
| | (S.D.) | | | | | |
| | Median | 0.0 | 0.0 | 0.0 | 0.0 | 0.0 |
| | Min-Max | −4.0-2.0 | −4.0-2.0 | −3.0-1.0 | −3.0-2.0 | −3.0-1.0 |
| | Trt Effect | −0.31 | 0.12 | −0.19 | | |
| | Trt Effect | (−0.56, 0.07) | (−0.13, 0.36) | (−0.44, 0.05) | | |
| | CI | | | | | |
| | P-value | 0.013* | 0.351 | 0.122 | | |

*Significant at the 0.050 level.
Note:
Pairwise comparisons placebo are based on an ANCOVA model with treatment (excluding risperidone) and pooled center as fixed factors and baseline CGI Severity of Illness score as a covariate.

CLINICAL STUDY TWO: Table 25 presents mean CGI-S scores at Baseline and the mean change from Baseline by visit using LOCF for the ITT population (LOCF). The mean change (SD) from Baseline to Endpoint in CGI severity of illness score was −0.69 (1.19) for the 30 mg bifeprunox group, −0.54 (1.12) for the 40 my bifeprunox group, −0.37 (1.07) for the placebo group, and −1.06 (1.20) for the risperidone group. The treatment effect values (bifeprunox—placebo) at Endpoint LOCF were: −0.28 for the 30 mg bifeprunox group and −0.18 for the 40 mg bifeprunox group. No statistically significant difference in change from Baseline to Endpoint in CGI-S was seen for the 30 mg bifeprunox treatment group compared to placebo based on the step-down procedure (adjusted p=0.056). The 30 mg bifeprunox group showed a notable difference from placebo at endpoint (nominal p=0.028). Notable differences between 30 mg bifeprunox and placebo treatment groups were also noted at Week 2 through Week 4 (p≦0.015). No differences were observed between placebo and the 40 mg bifeprunox dose group at any time point during the study (Week 1 through Week 4).

TABLE 25
Change from Baseline in CGI Severity of Illness Score-Last
Observation Carried Forward at Each Visit Intent-to-Treat Population.

| | | Treatment Group | | | |
|---|---|---|---|---|---|
| | | Bifeprunox | Bifeprunox | | Risperidone |
| | Statistic | 30 mg | 40 mg | Placebo | 6 mg |
| Total Number of | N | 140 | 141 | 144 | 151 |
| Subjects in the ITT | | | | | |
| Population | | | | | |
| Baseline | N | 140 | 141 | 144 | 151 |
| | Mean (SD) | 4.78 (0.65) | 4.67 (0.66) | 4.66 (0.68) | 4.72 (0.71) |
| | Median | 5.0 | 5.0 | 5.0 | 5.0 |
| | Min, Max | 4.0, 6.0 | 4.0, 6.0 | 4.0, 6.0 | 4.0, 6.0 |
| Change from Baseline | | | | | |
| Week 1 | N | 139 | 139 | 144 | 149 |
| | Mean (SD) | −0.33 (0.77) | −0.28 (0.65) | −0.19 (0.66) | −0.39 (0.63) |
| | Median | 0.0 | 0.0 | 0.0 | 0.0 |
| | Min, Max | −3.0, 2.0 | −3.0, 1.0 | −4.0, 2.0 | −3.0, 1.0 |
| | P-value | 0.171 | 0.249 | | |
| Week 2 | N | 140 | 140 | 144 | 151 |
| | Mean (SD) | −0.58 (0.97) | −0.43 (0.90) | −0.28 (0.83) | −0.73 (0.95) |
| | Median | 0.0 | 0.0 | 0.0 | −1.0 |
| | Min, Max | −3.0, 2.0 | −3.0, 2.0 | −30, 2.0 | −3.0, 1.0 |
| | P-value | 0.009 | 0.110 | | |
| Week 3 | N | 140 | 140 | 144 | 151 |
| | Mean (SD) | −0.67 (1.08) | −0.54 (1.02) | −0.34 (1.00) | −0.94 (1.00) |
| | Median | 0.0 | 0.0 | 0.0 | −1.0 |
| | Min, Max | −3.0, 2.0 | −3.0, 2.0 | −4.0, 2.0 | −4.0, 2.0 |
| | P-value | 0.014 | 0.074 | | |
| Week 4 | N | 140 | 140 | 144 | 151 |
| | Mean (SD) | −0.68 (1.11) | −0.51 (1.08) | −0.34 (1.03) | −1.02 (1.09) |
| | Median | 0.0 | 0.0 | 0.0 | −1.0 |
| | Min, Max | −4.0, 2.0 | −3.0, 2.0 | −4.0, 2.0 | −5.0, 2.0 |
| | P-value | 0.015 | 0.119 | | |
| Week 6/Endpoint | N | 140 | 141 | 144 | 151 |
| | Mean (SD) | −0.69 (1.19) | −0.54 (1.12) | −0.37 (1.07) | −1.06 (1.20) |
| | Median | 0.0 | 0.0 | 0.0 | −1.0 |
| | Min, Max | −4.0, 2.0 | −4.0, 2.0 | −4.0, 2.0 | −5.0, 2.0 |
| | Trt Effect | −0.28 | −0.18 | | |
| | 95% CI | −0.53, −0.03 | −0.43, 0.07 | | |
| | 97.5% CI | −0.06, 0.0 | 0.5, 0.1 | | |
| | P-value (Raw) | 0.028 | 0.149 | | |
| | P-value (Adj) | 0.056 | | | |

Note:
Treatment comparisons versus placebo were based an ANCOVA model with treatment (excluding risperidone and center as fixed factors and Baseline CGI severity of illness score as a covariate.
Note:
Adjusted p-value was based on the Hochberg procedure for multiple comparisons.
Note:
P-values presented for Week 1 through Week 4 are for descriptive purposes only.
Trt = Treatment,
Adj = Adjusted.

CLINICAL STUDY THREE: Table 26 below presents mean CGI Severity of Illness scores at Baseline and the mean change from Baseline by visit using LOCF for the ITT population. The mean change (SD) from Baseline to Endpoint in CGI severity of illness score was −0.63 (0.98) for the 20 mg bifeprunox group, −0.62 (1.03) for the 30 mg bifeprunox group, −0.49 (1.06) for the placebo group, and −1.03 (1.00) for the olanzapine group. The treatment effect values (bifeprunox—placebo) at Endpoint LOCF were: −0.13 for the 20 mg bifeprunox group and −0.09 for the 30 mg bifeprunox group. No differences were notable with nominal p-values (≦0.05) between the placebo group and either of the bifeprunox dose groups at Week 6/Endpoint or at any other time point during the study (Week 1 through Week 4).

TABLE 26
Change from Baseline in CGI Severity of Illness Score-Last
Observation Carried Forward at Each Visit Intent-to-Treat Population.

| | | Treatment Group | | | |
|---|---|---|---|---|---|
| | | Bifeprunox | Bifeprunox | | Olanzapine |
| | Statistic | 20 mg | 30 mg | Placebo | 15 mg |
| Total Number of Subjects | N | 149 | 148 | 145 | 146 |
| in | | | | | |
| the ITT Population | | | | | |
| Baseline | n | 149 | 148 | 145 | 146 |
| | Mean (SD) | 4.75 (0.69) | 4.80 (0.69) | 4.70 (0.64) | 4.86 (0.71) |
| | Median | 5.0 | 5.0 | 5.0 | 5.0 |
| | Min, Max | 4.0, 6.0 | 3.0, 6.0 | −4.0, 6.0 | 4.0, 7.0 |
| Change from Baseline | | | | | |
| Week 1 | n | 149 | 147 | 144 | 146 |
| | Mean (SD) | −0.23 (0.63) | −0.26 (0.60) | −0.27 (0.61) | −0.40 (0.65) |
| | Median | 0.0 | 0.0 | 0.0 | 0.0 |
| | Min, Max | −2.0, 2.0 | −2.0, 1.0 | −2.0, 1.0 | −3.0, 1.0 |
| | P-value | 0.507 | 0.554 | | |
| Week 2 | n | 149 | 148 | 145 | 146 |
| | Mean (SD) | −0.48 (0.81) | −0.41 (0.76) | −0.46 (0.83) | −0.71 (0.76) |
| | Median | 0.0 | 0.0 | 0.0 | −1.0 |
| | Min, Max | −3.0, 2.0 | −2.0, 10 | −30, 1.0 | −3.0, 1.0 |
| | P-value | 0.995 | 0.228 | | |
| Week 3 | n | 149 | 148 | 145, | 146 |
| | Mean (SD) | −0.59 (0.89) | −0.50 (0.90) | −0.54 (0.89) | −0.85 (0.87) |
| | Median | −1.0 | 0.0 | 0.0 | −1.0 |
| | Min, Max | −3.0, 2.0 | −3.0, 2.0 | −30, 2.0 | −4.0, 1.0 |
| | P-value | 0.681 | 0.465 | | |
| Week 4 | n | 149 | 148 | 145 | 146 |
| | Mean (SD) | −0.65 (0.95) | −0.57 (0.89) | −0.54 (0.96) | −0.92 (0.90) |
| | Median | −1.0 | −1.0 | 0.0 | −1.0 |
| | Min, Max | −30, 2.0 | −3.0, 2.0 | −3.0, 2.0 | −3.0, 1.0 |
| | P-value | 0.349 | 0.999 | | |
| Week 6/Endpoint | n | 149 | 148 | 145 | 146 |
| | Mean (SD) | −0.63 (0.98) | −0.62 (1.03) | −0.49 (1.06) | −1.03 (1.00) |
| | Median | −1.0 | −1.0 | 0.0 | −1.0 |
| | Min, Max | −4.0, 2.0 | −4.0, 2.0 | −4.0, 2.0 | −4.0, 1.0 |
| | Trt Effect | −0.13 | −0.09 | | |
| | Trt Effect | −0.35, 0.09 | −0.31, 0.13 | | |
| | 95% CI | | | | |
| | P-value | 0.262 | 0.434 | | |

Note:
Treatment comparisons versus placebo were based on an ANCOVA model with treatment (excluding olanzapine) and pooled center as fixed factors and Baseline CGI severity of illness score as a covariate.
Note:
Significance at Week 6/Endpoint for multiple comparisons was evaluated according to the Hochberg procedure.
Note:
P-values and CIs presented for Week 1 through Week 4, and Week 6/Endpoint are for descriptive purposes only.
Trt = Treatment.

### CGI-I

CLINICAL STUDY ONE: Improvement was noted in the CGI improvement scores for the 20 mg bifeprunox treatment groups as shown in Table 27. The treatment group difference was statistically significant at Week 1 (p=0.040) and Week 2 (p=0.016) for the bifeprunox 20 mg group versus placebo in the LOCF analysis, but not at Endpoint. No other significant differences were noted.

TABLE 27
Actual Values of CGI Improvement Score-Last Observation Carried
Forward at Each Visit Intent-to-Treat Population.

| | | Treatment Group | | | | |
|---|---|---|---|---|---|---|
| | | Bifeprunox | Bifeprunox | Bifeprunox | | Risperidone |
| | Statistic | 5 mg | 10 mg | 20 mg | Placebo | 6 mg |
| Total Number of | N | 110 | 118 | 111 | 114 | 116 |
| Subjects in the | | | | | | |
| ITT Population | | | | | | |
| Actual Value | | | | | | |
| Week 1 | n | 110 | 117 | 111 | 114 | 115 |
| | Mean (S.D.) | 3.76 (0.85) | 3.88 (1.00) | 3.54 (0.99) | 3.77 (0.93) | 3.43 (0.97) |
| | Median | 4.0 | 4.0 | 4.0 | 4.0 | 3.0 |
| | Min-Max | 1.0-6.0 | 1.0-6.0 | 1.0-6.0 | 2.0-6.0 | 1.0-6.0 |
| | P-value | 0.934 | 0.382 | 0.040* | | |
| Week 2 | n | 110 | 117 | 111 | 114 | 115 |
| | Mean (S.D.) | 3.46 (1.10) | 3.71 (1.23) | 3.30 (114) | 3.65 (1.18) | 3.04 (1.0)5 |
| | Median | 3.0 | 4.0 | 3.0 | 4.0 | 3.0 |
| | Min-Max | 1.0-6.0 | 1.0-6.0 | 1.0-6.0 | 1.0-6.0 | 1.0-6.0 |
| | P-value | 0.210 | 0.714 | 0.016* | | |
| Week 3 | n | 110 | 117 | 111 | 114 | 115 |
| | Mean (S.D.) | 3.2 (1.27) | 3.79 (1.32) | 3.31 (1.19) | 3.59 (1.30) | 2.97 (1.05) |
| | Median | 3.0 | 4.0 | 3.0 | 3.0 | 3.0 |
| | Min-Max | 1.0-6.0 | 1.0-6.0 | 1.0-6.0 | 1.0-6.0 | 1.0-6.0 |
| | P-value | 0.279 | 0.243 | 0.058 | | |
| Week 4 | n | 110 | 117 | 111 | 114 | 115 |
| | Mean (S.D.) | 3.37 (1.35) | 3.86 (1.35) | 3.35 (1.29) | 3.53 (1.39) | 3.05 (1.11) |
| | Median | 3.0 | 4.0 | 3.0 | 4.0 | 3.0 |
| | Min-Max | 1.0-6.0 | 1.0-6.0 | 1.0-6.0 | 1.0-6.0 | 1.0-6.0 |
| | P-value | 0.351 | 0.063 | 0.220 | | |
| Endpoint LOCF | n | 110 | 117 | 111 | 114 | 115 |
| | Mean (S.D.) | 3.33 (1.37) | 3.90 (1.39) | 342 (1.30) | 3.61 (1.41) | 3.02 (1.21) |
| | Median | 3.0 | 4.0 | 3.0 | 4.0 | 3.0 |
| | Trt Effect | −0.28 | 0.28 | −0.21 | | |
| | Trt Effect CI | (−0.62, 0.06) | (−0.06, 0.62) | (−0.55, 0.14) | | |
| | P-value | 0.110 | 0.109 | 0.235 | | |

*Significant at the 0.050 level.
Note:
Pairwise comparisons versus placebo are based on an ANCOVA model with treatment (excluding risperidone) and pooled center as fixed factors and baseline CGI
Severity of Illness score as a covariate.

CLINICAL STUDY TWO: The percentages of subjects who reported much or very much improvement from Baseline to Endpoint (LOCF) were 36% in the 30 mg bifeprunox group, 28% in the 40 mg bifeprunox group, 25% in the placebo group, and 52% in the risperidone group (Table 28). At Endpoint, no notable differences were observed between placebo and either of the two bifeprunox dose groups; however, notable differences between the 30 mg bifeprunox group and placebo were seen at Week 2 through Week 4 (pS0.024). No notable differences were seen between the 40 mg bifeprunox group and placebo at any time point during the study (Week 1 through Endpoint). Data for Week 2 through Endpoint are summarized in the Table below.

TABLE 28
CGI Improvement Score-Last Observation Carried Forward at Each
Visit Intent-to-Treat Population.

| | Treatment Group | | | | | | | |
|---|---|---|---|---|---|---|---|---|
| | Bifeprunox | | Bifeprunox | | | | Risperidone | |
| | 30 mg | | 40 mg | | Placebo | | 6 mg | |
| Total Number of Subjects in the | 140 | | 141 | | 144 | | 151 | |
| ITT Population, n | | | | | | | | |
| Week 2, n | 140 | | 140 | | 144 | | 151 | |
| Not Assessed, n (%) | 0 | | 0 | | 0 | | 0 | |
| Very Much Improved, n (%) | 4 | (3%) | 5 | (4%.) | 6 | (4%) | 8 | (5%) |
| Much Improved, n (%) | 40 | (29%) | 27 | (19%) | 18 | (13%) | 45 | (30%) |
| Minimally Improved, n (%) | 46 | (33%) | 52 | (37%) | 46 | (32%) | 61 | (40%) |
| No Change, n (%) | 34 | (24%) | 31 | (22%) | 41 | (29%) | 24 | (16%) |
| Minimally Worse, n (%) | 11 | (8%) | 10 | (7%) | 16 | (11%) | 8 | (5%) |
| Much Worse, n (%) | 4 | (3%) | 15 | (11%) | 15 | (10%) | 5 | (3%) |
| Very Much Worse, (%) | 1 | (<1%) | 0 | | 1 | (<1%) | 0 | |
| P-value | 0.001** | | 0.059 | | | | | |
| Week 3, n | 140 | | 140 | | 143 | | 151 | |
| Not Assessed, n (%) | 0 | | 0 | | 0 | | 0 | |
| Very Much Improved, n (%) | 9 | (6%) | 7 | (5%) | 9 | (6%) | 13 | (9%) |
| Much Improved, n (%) | 38 | (27%) | 30 | (21%) | 21 | (15%) | 55 | (36%) |
| Minimally Improved, n (%) | 39 | (28%) | 42 | (30%) | 44 | (31%) | 50 | (33%) |
| No Change, n (%) | 37 | (26%) | 32 | (23%) | 34 | (24%) | 19 | (13%) |
| Minimally Worse, n (%) | 10 | (7%) | 9 | (6%) | 16 | (11%) | 7 | (5%) |
| Much Worse, n (%) | 6 | (4%) | 20 | (14%) | 18 | (13%) | 7 | (5%) |
| Very Much Worse, (%) | 1 | (<1%) | 0 | | 1 | (<1%) | 0 | |
| P-value | 0.012* | | 0.256 | | | | | |
| Week 4, n | 140 | | 140 | | 143 | | 151 | |
| Not Assessed, n (%) | 0 | | 0 | | 0 | | 0 | |
| Very Much Improved, n (%) | 12 | (9%) | 10 | (7%) | 9 | (6%) | 20 | (13%) |
| Much Improved, n (%) | 37 | (26%) | 28 | (20%) | 27 | (19%) | 58 | (38%) |
| Minimally Improved, n (%) | 37 | (26%) | 40 | (29%) | 36 | (25′%) | 37 | (25%) |
| No Change, n (%) | 34 | (24%) | 29 | (21%) | 36 | (25%) | 21 | (14%) |
| Minimally Worse, n (%) | 13 | (9%) | 13 | (9%) | 15 | (10%) | 8 | (5%) |
| Much Worse, n (%) | 6 | (4%) | 20 | (14%) | 19 | (13%) | 7 | (5%) |
| Very Much Worse, (%) | 1 | (<1%) | 0 | | 1 | (<1%) | 0 | |
| P-value | 0.024 | | 0.345 | | | | | |
| Week 6/Endpoint, n | 140 | | 141 | | 143 | | 151 | |
| Not Assessed, n (%) | 0 | | 0 | | 1 | (<1%) | 0 | |
| Very Much Improved, n (%) | 12 | (9%) | 13 | (9%) | 6 | (4%) | 24 | (16%) |
| Much Improved, n (%) | 39 | (28%) | 27 | (19%) | 30 | (21%) | 54 | (36%) |
| Minimally Improved, n (%) | 30 | (21%) | 37 | (26%) | 40 | (28%) | 34 | (23%) |
| No Change, n (%) | 34 | (24%) | 28 | (20%) | 31 | (22%) | 23 | (15%) |
| Minimally Worse, n (%) | 16 | (11%) | 15 | (11%) | 16 | (11%) | 7 | (5%) |
| Much Worse, n (%) | 8 | (6%) | 21 | (15%) | 19 | (13%) | 8 | (5%) |
| Very Much Worse, (%) | 1 | (<1%) | 0 | | 1 | (<1%) | 1 | (<1%) |

| | | |
|---|---|---|
| P-value | 0.079 | 0.452 |

*Notably different from placebo, p ≦ 0.050.
**Notably different from placebo, p ≦ 0.010.
Note:
P-values were based on a CMH test stratified by pooled center.

CLINICAL STUDY THREE: Table 29 below presents frequencies for the seven categories of CGI Improvement ratings by visit using LOCF for the ITT population. Statistical differences among treatment groups regarding the mean CGI Improvement score were evaluated using a CMH test stratified by pooled center applied to the frequencies of the seven CGI Improvement categories with modified ridit scores. The percentages of subjects who reported much or very much improvement combined, (derived by summing the corresponding individual percentages in Table 23) from Baseline to Endpoint were: 38% in the 20 mg bifeprunox group, 34% in the 30 mg bifeprunox group, 32% in the placebo group, and 46% in the olanzapine group. Comparison of the 20 mg bifeprunox group versus placebo group at Endpoint was found to be notable (p=0.027). The treatment comparison of the 30 mg bifeprunox group versus placebo group was not notable (p=0.162). No notable differences were observed between the placebo group and either of the two bifeprunox dose groups at any other time point during the study (Week 1 through Week 4).

TABLE 29
CGI Improvement Score-Last Observation Carried Forward at Each
Visit Intent-to-Treat Population.

| | Treatment Group | | | | | | | |
|---|---|---|---|---|---|---|---|---|
| | Bifeprunox | | Bifeprunox | | | | Olanzapine | |
| | 30 mg | | 40 mg | | Placebo | | 15 mg | |
| Total Number of Subjects in the | 149 | | 148 | | 145 | | 146 | |
| ITT Population, n | | | | | | | | |
| Week 2, n | 149 | | 148 | | 145 | | 146 | |
| Not Assessed, n (%) | 0 | | 0 | | 0 | | 0 | |
| Very Much Improved, n (%) | 3 | (2%) | 1 | (<1%.) | 4 | (3%) | 6 | (4%) |
| Much Improved, n (%) | 32 | (21%) | 31 | (19%) | 34 | (23%) | 39 | (27%) |
| Minimally Improved, n (%) | 67 | (45%) | 55 | (37%) | 49 | (34%) | 65 | (45%) |
| No Change, n (%) | 28 | (19%) | 33 | (22%) | 35 | (24%) | 26 | (18%) |
| Minimally Worse, n (%) | 13 | (9%) | 11 | (7%) | 18 | (12%) | 9 | (6%) |
| Much Worse, n (%) | 6 | (4%) | 15 | (10%) | 5 | (3%) | 1 | (<1%) |
| Very Much Worse, (%) | 0 | | 1 | (<1%) | 0 | | 0 | |
| P-value | 0.334 | | 0.661 | | | | | |
| Week 4, n | 149 | | 148 | | 145 | | 146 | |
| Not Assessed, n (%) | 0 | | 0 | | 0 | | 0 | |
| Very Much Improved, n (%) | 8 | (5%) | 5 | (3%) | 10 | (7%) | 10 | (7%) |
| Much Improved, n (%) | 47 | (32%) | 45 | (30%) | 38 | (26%) | 53 | (36%) |
| Minimally Improved, n (%) | 44 | (30%) | 43 | (29%) | 28 | (19%) | 43 | (29%) |
| No Change, n (%) | 25 | (17%) | 25 | (17%) | 29 | (20%) | 25 | (17%) |
| Minimally Worse, n (%) | 16 | (11%) | 12 | (8%) | 32 | (22%) | 10 | (7%) |
| Much Worse, n (%) | 9 | (6%) | 16 | (11%) | 8 | (6%) | 5 | (3%) |
| Very Much Worse, (%) | 0 | | 2 | (1%) | 0 | | 0 | |
| P-value | 0.057 | | 0.363 | | | | | |
| Week 6/Endpoint, n | 149 | | 148 | | 145 | | 146 | |
| Not Assessed, n (%) | 0 | | 0 | | 0 | | 0 | |
| Very Much Improved, n (%) | 7 | (5%) | 10 | (7%) | 9 | (6%) | 17 | (12%) |
| Much Improved, n (%) | 49 | (33%) | 40 | (27%) | 37 | (26%) | 50 | (34%) |
| Minimally Improved, n (%) | 42 | (28%) | 44 | (30%) | 27 | (19%) | 43 | (29%) |
| No Change, n (%) | 22 | (15%) | 23 | (16%) | 31 | (21%) | 21 | (14%) |
| Minimally Worse, n (%) | 19 | (13%) | 12 | (8%) | 31 | (21%) | 10 | (7%) |
| Much Worse, n (%) | 10 | (7%) | 17 | (11%) | 10 | (7%) | 5 | (3%) |
| Very Much Worse, (%) | 0 | | 2 | (1%) | 0 | | 10 | |

| | | |
|---|---|---|
| P-value | 0.027* | 0.162 |

*Notably different from placebo, p ≦ 0.050.
Note:
P-values were based on a CMH test stratified by pooled center.

### PANSS Responder Rates

CLINICAL STUDY ONE: The PANSS responder rates were higher for each of the bifeprunox treatment groups compared to the placebo group. The PANSS responder rates were 28%, 24%, and 34% for the three dose groups respectively using the 20% definition from the study protocol. Responder rates for PANSS using all four definitions are presented in the Table 30 below.

TABLE 30
PANSS Responder Rates at Endpoint Last Observation Carried
Forward Intent-to-Treat Population.

| | | Treatment Group | | | | |
|---|---|---|---|---|---|---|
| | | Bifeprunox | Bifeprunox | Bifeprunox | | Risperidone |
| | Statistic | 5 mg | 10 mg | 20 mg | Placebo | 6 mg |
| Total | N | 110 | 118 | 111 | 114 | 116 |
| Number of | | | | | | |
| Subjects in | | | | | | |
| the ITT | | | | | | |
| Population | | | | | | |

PANSS Responder at Endpoint (LOCF)

| | | | | | | |
|---|---|---|---|---|---|---|
| 20% | n (%) | 31 (28) | 28 (24) | 38 (34) | 25 (22) | 46 (40) |
| Definition | | | | | | |
| | P-value | 0.314 | 0.807 | 0.052 | | |
| 25% | n (%) | 25 (23) | 22 (19) | 27 (24) | 12 (11) | 34 (29) |
| Definition | | | | | | |
| | P-value | 0.021* | 0.094 | 0.008** | | |
| 30% | n (%) | 16 (15) | 12 (10) | 18 (16) | 8 (7) | 26 (22) |
| Definition | | | | | | |
| | P-value | 0.093 | 0.400 | 0.042* | | |
| 35% | n (%) | 11 (10) | 7 (6) | 14 (13) | 4 (4) | 15 (13) |
| Definition | | | | | | |
| | P-value | 0.051 | 0.357 | 0.013* | | |

*Significant at the 0.050 level.
**Significant at the 0.010 level.
Note:
A PANSS responder is defined as a subject whose PANSS total score decreased by the specified percentage.

A significant difference in the PANSS responder rate using LOCF was seen using the 25% definition for bifeprunox 5 mg versus placebo, and for the 25%, 30%, and 35% definitions for the bifeprunox 20 mg groups versus placebo.

CLINICAL STUDY TWO: A PANSS responder was defined as a subject whose PANSS total score decreased by 20% or more from Baseline to Endpoint based on LOCF data. As exploratory analyses, responder rates were also analyzed using a 30%, 35%, 40%, and 50% definition of responder. Summaries of the PANSS responder rates at Endpoint (LOCF) are presented in the Table 31 below.

The PANSS responder rates were slightly higher for the two bifeprunox treatment groups compared to the placebo group. The PANSS 20% responder rates were: 36% in the 30 mg bifeprunox group, 30% in the 40 mg bifeprunox group, 26% in the placebo group, and 54% in the risperidone group. However, the difference between 30 mg bifeprunox group and placebo was not notable (p=0.052). The PANSS 30% responder rates were: 24% in the 30 mg bifeprunox group, 22% in the 40 mg bifeprunox group, 14% in the placebo group, and 31% in the risperidone group. The difference between the 30 mg bifeprunox group and placebo was notable (p=0.019). Notable differences between 30 mg bifeprunox group and placebo were also seen for 35% and 50% responder rates.

TABLE 31
PANSS Responder Rate At Endpoint Last Observation Carried
Forward Intent-to-Treat Population.

| | Treatment Group | | | |
|---|---|---|---|---|
| | Bifeprunox | Bifeprunox | | Risperidone |
| | 30 mg | 40 mg | Placebo | 6 mg |
| Total Number of | 140 | 141 | 144 | 151 |
| Subjects in the | | | | |
| ITT Population, | | | | |

PANSS Responder at Endpoint

| | | | | |
|---|---|---|---|---|
| 20% Definition | | | | |
| Responder at | | | | |
| Week 6/ | | | | |
| Endpoint | | | | |
| Yes n (%) | 51 (36%) | 42 (30%) | 38 (26%) | 78 (54%) |
| No, n (%) | 89 (64%) | 99 (70%) | 106 (74%) | 69 (46%) |
| P-Value | 0.052 | 0.497 | | |
| 3% Definition | | | | |
| Responder | | | | |
| Yes n (%) | 34 (24%) | 31 (22%) | 20 (14%) | 47 (31%) |
| No, n (%) | 106 (76%) | 110 (78%) | 124 (86%) | 104 (69%) |
| P-Value | 0.039* | 0.483 | | |

*Notably different from placebo, p ≦ 0.050
Note:
p-values were based o the CMH test stratified by pooled center.
Note:
A PANSS responder was defined as a subject whose PANSS score decreased by ≧20% (20% definition) or ≧30% (30% definition)

CLINICAL STUDY THREE. A PANSS responder was defined as a subject whose PANSS total score decreased by 20% or more from Baseline to Endpoint. As exploratory analyses, responder rates were also analyzed using a 30%, 35%, 40%, and 50% definition of responder. Summaries of the PANSS responder rates at Endpoint (LOCF) are presented in the Table 32 below. The 20% PANSS responder rates were slightly higher for the two bifeprunox treatment groups compared to the placebo group: 42% of the subjects in the 20 mg bifeprunox group, 39% of the subjects in the 30 mg bifeprunox group, 32% of the subjects in the placebo group, and 54% of the subjects in the olanzapine group improved by 20% or more from Baseline to Endpoint in PANSS total score. The difference between the 20 mg bifeprunox group and the placebo group approached being notable (p=0.061). However, when more stringent criteria (30%-50%) for definition of responder were used, differences between the 20 mg or 30 mg bifeprunox groups and the placebo group were not notable (all p>0.118).

TABLE 32
PANSS Responder Rates at Endpoint Last Observation Carried
Forward Intent-to-Treat Population.

| | Treatment Group | | | |
|---|---|---|---|---|
| | Bifeprunox | Bifeprunox | | Olanzapine |
| | 20 mg | 30 mg | Placebo | 15 mg |
| Total Number of | 149 | 148 | 145 | 146 |
| Subjects in the | | | | |
| ITT Population | | | | |
| 20% definition | | | | |
| response rate | | | | |
| Week 6/Endpoint | | | | |
| Yes | 62 (42%) | 57 (39%) | 46 (32%) | 79 (54%) |
| No | 87 (58%) | 91 (61%) | 99 (68%) | 67 (46%) |
| P-value | 0.061 | 0.185 | | |

Note:
P values were based on CMH test stratified by pooled center.
Note:
A PANSS responder was defined as a subject whose PANSS score decreased by ≧20% from Baseline to Endpoint.

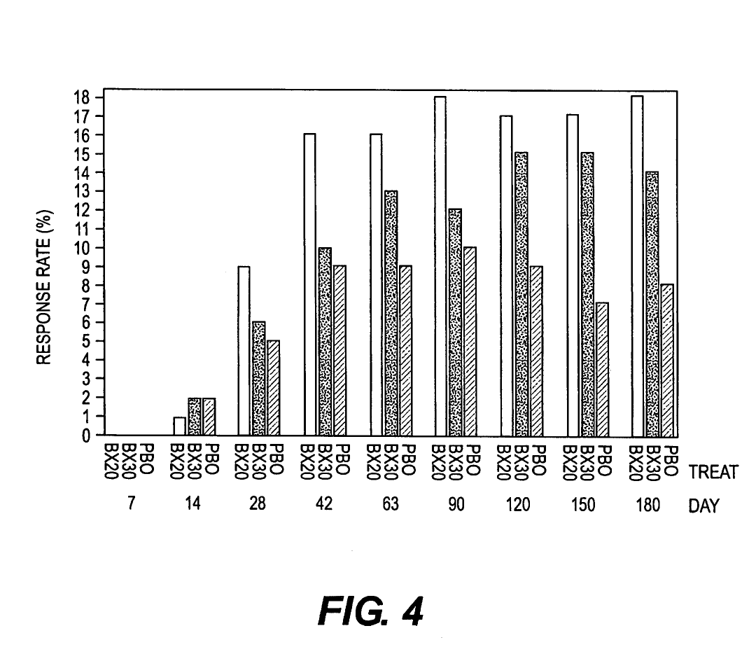

CLINICAL STUDY FOUR: The proportion of patients with a ≧25%, ≧35%, ≧45%, or ≧55% reduction in PANSS total score relative to baseline at each visit (PANSS responders) is shown by LOCF (FIG. 4).

### CGI Responder Rates

CLINICAL STUDY ONE: A CGI responder is defined as a subject who is categorized as “very much improved” or “much improved” on the CGI Improvement scale. The CGI responder rates for the bifeprunox 5 mg and 20 mg groups were higher than the responder rates for the placebo group, as provided in Table 33 below. No statistically significant differences were seen for any of the three bifeprunox dose groups when compared to placebo.

TABLE 33
CGI Responder Rate at Endpoint Last Observation Carried Forward
at Each Visit Intent-to-Treat Population.

| | | Treatment Group | | | | |
|---|---|---|---|---|---|---|
| | | Bifeprunox | Bifeprunox | Bifeprunox | | Risperidone |
| | Statistic | 5 mg | 10 mg | 20 mg | Placebo | 6 mg |
| Total Number | N | 110 | 118 | 111 | 114 | 116 |
| of | | | | | | |
| Subjects in | | | | | | |
| the ITT | | | | | | |
| Population | | | | | | |
| CGI | n (%) | 36 (33) | 22 (19) | 28 (25) | 25 (22) | 41 (36) |
| Responder at | | | | | | |
| Endpoint | | | | | | |
| (LOCF) | | | | | | |
| | P-value | 0.065 | 0.521 | 0.513 | | |

Note:
A CGI responder is defined as a subject who is categorized as “very much improved” or “much improved” in the CGI Improvement scale.
Note:
P-values are based on a Cochran-Mantel-Haenszel chi-square test stratified by pooled center.

CLINICAL STUDY TWO: A CGI responder is defined as a subject who is categorized as “very much improved” or “much improved” on the CGI Improvement scale. Summaries of CGI-I responder rates at Endpoint (LOCF) are presented in the Table 34 below. The CGI-I responder rates were: 36% in the 30 mg bifeprunox group, 28% in the 40 mg bifeprunox group, 25% in the placebo group, and 52% in the risperidone group. A notable difference between the 30 mg bifeprunox group and placebo was seen (p=0.039).

TABLE 34
CGI Responder Rate At Endpoint Last Observation Carried Forward
Intent-to-Treat Population.

| | Treatment Group | | | |
|---|---|---|---|---|
| | Bifeprunox | Bifeprunox | | Risperidone |
| | 30 mg | 40 mg | Placebo | 6 mg |
| Total Number of Subjects in the | 140 | 141 | 144 | 151 |
| ITT Population, n | | | | |
| CGI Responder at Endpoint, n | 140 | 141 | 143 | 151 |
| Yes n (%) | 51 (36%) | 40 (28%) | 36 (25%) | 78 (52%) |
| No, n (%) | 89 (64%) | 101 (72%) | 107 (75%) | 73 (48%) |
| P-Value | 0.039* | 0.483 | | |

*Notably different from placebo, p ≦ 0.050.
Note:
p-values were based on the CMH test stratified by pooled center.
Note:
A CGI responder was defined as a subject who was categorized as “very much improved” or “much improved” in the CGI improvement scale.

CLINICAL STUDY THREE: A CGI responder is defined as a subject who is categorized as “very much improved” or “much improved” on the CGI Improvement scale. Summaries of CGI-I responder rates at Endpoint (LOCF) are presented in Table 35. The CGI-I responder rates were: 38% in the 20 mg bifeprunox group, 34% in the 30 mg bifeprunox group, 32% in the placebo group, and 46% in the olanzapine group. No notable differences were observed between the placebo group and either of the two bifeprunox dose groups.

TABLE 35
CGI Responder Rate at Endpoint Last Observation Carried
Forward Intent-to-Treat Population.

| | Treatment Group | | | |
|---|---|---|---|---|
| | Bifeprunox | Bifeprunox | | Olanzapine |
| | 20 mg | 30 mg | Placebo | 15 mg |
| Total Number of | 149 | 148 | 145 | 146 |
| Subjects in the | | | | |
| ITT Population | | | | |
| Week 6/ | 149 | 148 | 145 | 146 |
| Endpoint, n | | | | |
| Yes | 56 (38%) | 50 (34%) | 46 (32%) | 67 (46%) |
| No | 93 (62%) | 93 (66%) | 99 (68%) | 79 (54%) |
| P-value | 0.264 | 0.674 | | |

Note:
P values were based on a CMH test stratified by pooled center.
Note:
A CGI responder was defined as a subject who was categorized as “very much improved or “much improved” in the CGI improvement scale.

### CDSS

CLINICAL STUDY TWO. Calgary Depression Scale for Schizophrenia scores are presented in the Table 36 below. This Table presents mean CDSS scores at Baseline and the mean change from Baseline by visit using LOCF for the ITT population. The mean change (SD) from Baseline to Endpoint in CDSS score (LOCF) was −0.66 (3.64) for the 30 mg bifeprunox group, 0.01 (3.66) for the 40 mg bifeprunox group, −0.39 (3.45) for the placebo group, and −0.89 (3.42) for the risperidone group. The treatment effect values (bifeprunox, placebo) at Endpoint LOCF were: −0.38 for the 30 mg bifeprunox group and 0.29 for the 40 mg bifeprunox group. No notable differences were observed between placebo and either of the two bifeprunox dose groups at Endpoint or at any other timepoint during the study (Week 1 through Week 4).

TABLE 36
Change from Baseline in CDSS Total Score-Last Observation
Carried Forward at Each Visit Intent-to-Treat Population.

| | | Treatment Group | | | |
|---|---|---|---|---|---|
| | | Bifeprunox | Bifeprunox | | Risperidone |
| | Statistic | 30 mg | 40 mg | Placebo | 6 mg |
| Total Number of | N | 140 | 141 | 144 | 151 |
| Subjects in the ITT | | | | | |
| Population | | | | | |
| Baseline | N | 139 | 141 | 144 | 151 |
| | Mean (SD) | 3.63 (3.70) | 3.52 (4.04) | 3.80 (4.04) | 3.69 (3.82) |
| | Median | 2.0 | 2.0 | 2.5 | 3.0 |
| | Min, Max | 0.0, 16.0 | 0.0, 19.0 | 0.0, 17.0 | 0.0, 17.0 |
| Change from Baseline | | | | | |
| Week 1 | n | 139 | 138 | 143 | 149 |
| | Mean (SD) | −0.01 (2.90) | −0.54 (2.56) | −0.24 (2.84) | −0.62 (2.42) |
| | Median | 0.0 | 0.0 | 0.0 | 0.0 |
| | Min, Max | −9.0, 13.0 | −12.0, 6.0 | −7.0, 12.0 | −9.0, 10.0 |
| | P-value | 0.538 | 0.241 | | |
| Week 2 | n | 139 | 139 | 143 | 151 |
| | Mean (SD) | −0.51 (3.37) | −0.32 (2.95) | −0.31 (3.10) | −0.74 (2.71) |
| | Median | 0.0 | 0.0 | 0.0 | 0.0 |
| | Min, Max | −11.0, 13.0 | −11.0, 14.0 | −9.0, 10.0 | 31 8.0, 12.0 |
| | P-value | 0.431 | 0.769 | | |
| Week 3 | n | 139 | 139 | 143 | 151 |
| | Mean (SD) | −0.51 (3.56) | −0.44 (3.09) | −02.6 (3.60) | −0.93 (3.41) |
| | Median | 0.0 | 0.0 | 0.0 | 0.0 |
| | Min, Max | −13.0, 13.0 | −14.0, 14.0 | −10.0, 10.0 | −13.0, 14.0 |
| | P-value | 0.377 | 0.422 | | |
| Week 4 | n | 139 | 139 | 143 | 151 |
| | Mean (SD) | −0.63 (3.48) | −0.19 (3.32) | −0.31 (3.48) | −0.95 (3.08) |
| | Median | 0.0 | 0.0 | 0.0 | 0.0 |
| | Min, Max | −13.0, 13.0 | −14.0, 14.0 | −10.0, 10.0 | −9.0, 12.0 |
| | P-value | 0.249 | 0.969 | | |
| Week 6/Endpoint | n | 139 | 140 | 143 | 151 |
| | Mean (SD) | −0.66 (3.64) | 0.01 (3.66) | −0.39 (3.45) | −0.89 (3.42) |
| | Median | 0.0 | 0.0 | 0.0 | 0.0 |
| | Min, Max | −13.0, 13. | −14.0, 14. | −9., 10.0 | −10.0, 12.0 |
| | Trt Effect | −0.38 | 0.29 | | |
| | 95% CI | −1.11, 0.35 | −0.44, 1.02 | | |
| | P-value | 0.313 | 0.436 | | |

Note:
Treatment comparisons versus placebo were based on an ANCOVA model with treatment (excluding risperidone) and pooled center as fixed factors and Baseline CDSS total score as a covariate.
Trt = Treatment.

CLINICAL STUDY THREE: Table 37 below presents mean CDSS scores at Baseline and the mean change from Baseline by visit using LOCF for the ITT population. The mean change (SD) from Baseline to Endpoint in CDSS score was −1.30 (3.85) for the 20 mg bifeprunox group, −0.79 (3.02) for the 30 mg bifeprunox group, −0.59 (4.20) for the placebo group, and −1.47 (3.95) for the olanzapine group. The treatment effect values (bifeprunox, placebo) at Endpoint LOCF were: −0.54 for the 20 mg bifeprunox group and −0.34 for the 20 mg bifeprunox group. No notable differences were observed between the placebo group and either of the two bifeprunox dose groups at Endpoint or at any other time point during the study (Week 1 through Week 4).

TABLE 37
Change from Baseline in CDSS Total Score-Last Observation
Carried Forward Intent-to-Treat Population.

| | | Treatment Group | | | |
|---|---|---|---|---|---|
| | | Bifeprunox | Bifeprunox | | Olanzapine |
| | Statistic | 20 mg | 30 mg | Placebo | 15 mg |
| Total Number of | N | 149 | 48 | 145 | 146 |
| Subjects in the ITT | | | | | |
| Population | | | | | |
| Baseline | n | 149 | 148 | 145 | 146 |
| | Mean | 4.34 (4.12) | 3.73 (3.67) | 4.01 (3.88) | 3.99 (3.64) |
| | (SD) | | | | |
| | Median | 4.0 | 2.0 | 3.0 | 3.0 |
| | Min, Max | 0.0, 16.0 | 0.0, 15.0 | 0.0, 18.0 | 0.0, 14.0 |
| Change from | | | | | |
| Baseline | | | | | |
| Week 1 | n | 149 | 146 | 144 | 146 |
| | Mean | −0.86 (2.87) | −0.77 (2.63) | −0.60 (2.94) | −1-01 (2.91) |
| | (SD) | | | | |
| | Median | 0.0 | 0.0 | 0.0 | −1.0 |
| | Min, Max | −11.0, 8.0 | −10.0. 7.0 | −14.0, 8.0 | −12.0, 7.0 |
| | P-value | 0.633 | 0.375 | | |
| Week 2 | n | 149 | 147 | 145 | 146 |
| | Mean | −1.19 (3.30) | −0.69 (2.97) | −0.63 (3.73) | −1.33 (3.33) |
| | (SD) | | | | |
| | Median | −1.0 | 0.0 | 0.0 | −1.0 |
| | Min, Max | −11.0, 9.0 | −12.0, 13.0 | −16.0, 9.0 | −12.0, 12.0 |
| | P-value | 0.215 | 0.561 | | |
| Week 3 | n | 149 | 147 | 145 | 146 |
| | Mean | −1.41 (3.72) | −0.60 (3.10) | −1.12 (3.83) | −1.59 (3.43) |
| | (SD) | | | | |
| | Median | −1.0 | 0.0 | −1.0 | −1.0 |
| | Min, Max | −11.0, 9.0 | −11.0, 13.0 | −18.0, 11.0 | −11.0, 9.0 |
| | P-value | 0.730 | 0.275 | | |
| Week 4 | n | 149 | 147 | 145 | 146 |
| | Mean | −1.35 (3.66) | −0.65 (3.07) | −0.88 (4.08) | −1.49 (3.89) |
| | (SD) | | | | |
| | Median | −1.0 | 0.0 | −1.0 | −1.0 |
| | Min, Max | −12.0, 9.0 | −11.0, 13.0 | −18.0, 11.0 | −11.0, 9.0 |
| | P-value | 0.388 | 0.810 | | |
| Week 6, Endpoint | n | 149 | 147 | 145 | 146 |
| | Mean | −1.30 (3.85) | −0.79 (3.02) | −0.59 (4.20) | −1.47 (3.95) |
| | (SD) | | | | |
| | Median | 0.0 | −1.0 | 0.0 | −1.0 |
| | Min, Max | −11.0, 9.0 | −11-0, 13.0 | −18.0, 11.0 | −11.0, 9.0 |
| | Trt Effect | −0.54 | −0.34 | | |
| | Trt Effect | −1.26, 0.19 | −1.07, 354 | | |
| | CI | | | | |
| | P-value | 0.145 | 0.354 | | |

Note:
Treatment comparisons and least square means were based on an ANCOVA model with treatment (excluding olanzapine) and pooled center as fixed factors and Baseline
CDSS total score as a covariate.
Trt = Treatment.

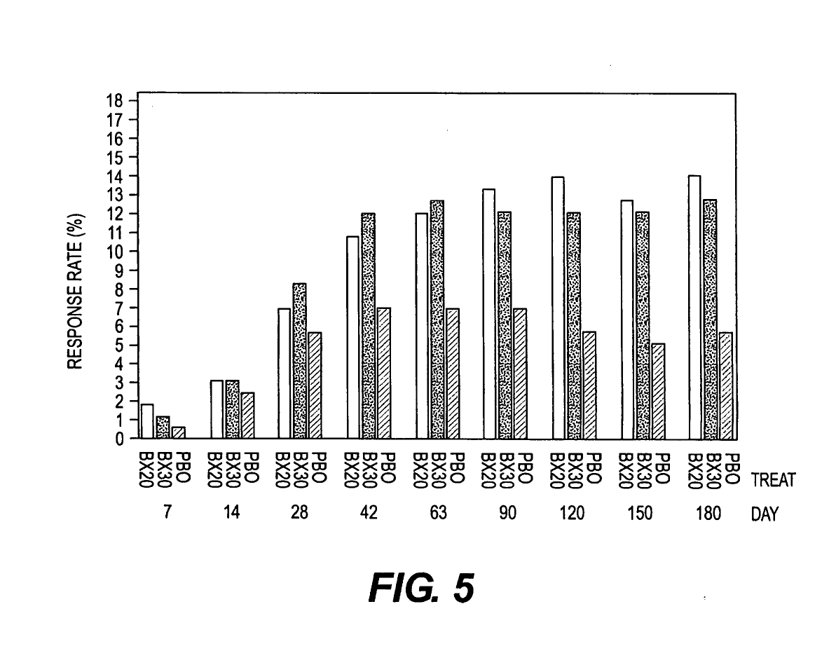

CLINICAL STUDY FOUR: The proportion of patients with a CGI-I score ≦2 at each visit (CGI-I responders; FAS, LOCF) is show below in FIG. 5.

### EPS

EPS were assessed using treatment-emergent adverse events such as akathisia, dyskinesia, parkinsoniam, etc. and/or formal rating scales such as SAS, BAS, and/or AIMS.

Simpson-Angus Scale 16 (SAS)

The SAS is used to measure Parkinsonian-type symptoms in patients exposed to antipsychotics. The scale consists of 10 items, each rated on a 5-point scale ranging from 0 (complete absence of the condition) to 4 (presence of the condition in extreme form). The SAS total score is defined as the sum of all item scores, and the range is 0 to 40. A SAS total score of up to 3 is considered normal.

Barnes Akathisia Scale 17 (BAS)

The BAS is used to rate observable, restless movements of drug-induced akathisia as well as the subjective awareness of restlessness and any distress associated with the akathisia. The scale consists of 3 items that is rated from 0 (no evidence of akathisia) to 3 (severe akathisia). The BAS total score is defined as the sum of the these three BAS item scores and ranges from is 0 to 9. In addition, a global clinical assessment of akathisia is rated from 0 (no evidence of akathisia) to 5 (severe akathisia).

Abnormal Involuntary Movement Scale 18 (AIMS)

The AIMS, designed to record the occurrence of dyskinetic movements, consists of 12 items. Items 1 to 7 measure specific involuntary movements on a scale from 0 (none) to 4 (severe). Items 8 to 10 measure global assessment of abnormal movement on a scale from 0 (no awareness) to 4 (aware, severe distress). Items 11 and 12 are questions regarding the dental condition of the patient, with yes/no answers. The total score is calculated by summing AIMS items 1 through 10 and ranges from 0 to 40; the non-global total score is calculated by summing items 1 through 7.

CLINICAL STUDY ONE: The following treatment-emergent adverse events were reported relating to EPS;

TABLE 38
Overall Incidence of Treatment-Emergent Adverse Events: Safety
Population.

| | | Treatment Group | | | | | |
|---|---|---|---|---|---|---|---|
| System Organ Class/ | | | | | | Risperidone | |
| Preferred Term | Statistic | BX 5 mg | BX 10 mg | BX 20 mg | Placebo | 6 mg | Overall |

MUSCULOSKELETAL AND CONNECTIVE TISSUE DISORDERS (cont'd)

| | | | | | | | | | | | | | |
|---|---|---|---|---|---|---|---|---|---|---|---|---|---|
| Muscle Spasms | n (%) | 0 | | 0 | | 2 | (2%) | 0 | | 2 | (2%) | 4 | (<1%) |
| Muscle Stiffness | n (%) | 1 | (<1%) | 1 | (<1%) | 0 | | 0 | | 0 | | 2 | (<1%) |
| Muscle Tightness | n (%) | 1 | (<1%) | 0 | | 0 | | 0 | | 0 | | 1 | (<1%) |
| Muscle Twitching | n (%) | 2 | (2%) | 1 | (<1%) | 1 | (<1%) | 0 | | 0 | | 4 | (<1%) |
| Musculoskeletal Discomfort | n (%) | 0 | | 1 | (<1%) | 0 | | 0 | | 0 | | 1 | (<1%) |
| Musculoskeletal Stiffness | n (%) | 0 | | 0 | | 0 | | 0 | | 1 | (<1%) | 1 | (<1%) |
| Myalgia | n (%) | 0 | | 1 | (<1%) | 1 | (<1%) | 1 | (<1%) | 2 | (2%) | 5 | (<1%) |
| Neck Pain | n (%) | 1 | (<1%) | 3 | (3%) | 2 | (2%) | 0 | | 2 | (2%) | 8 | (1%) |
| Neck Stiffness | n (%) | 0 | | 0 | | 0 | | 0 | | 1 | (<1%) | 1 | (<1%) |
| Pain in Jaw | n (%) | 1 | (<1%) | 1 | (<1%) | 0 | | 1 | (<1%) | 1 | (<1%) | 4 | (<1%) |
| Pain in Limb | n (%) | 1 | (<1%) | 2 | (2%) | 3 | (3%) | 4 | (3%) | 2 | (2%) | 12 | (2%) |
| Sensation of Heaviness | n (%) | 0 | | 0 | | 1 | (<1%) | 0 | | 0 | | 1 | (<1%) |
| NEOPLASMS BENIGN, | n (%) | 2 | (2%) | 0 | | 0 | | 0 | | 0 | | 2 | (<1%) |
| MALIGNANT AND | | | | | | | | | | | | | |
| UNSPECIFIED (INCL CYSTS | | | | | | | | | | | | | |
| AND POLYPS) | | | | | | | | | | | | | |
| Benign Skin Neoplasm Nos | n (%) | 1 | (<1%) | 0 | | 0 | | 0 | | 0 | | 1 | (<1%) |
| Skin Papilloma | n (%) | 1 | (<1%) | 0 | | 0 | | 0 | | 0 | | 1 | (<1%) |
| NERVOUS SYSTEM | n (%) | 55 | (48%) | 60 | (50%) | 49 | (43%) | 52 | (44%) | 59 | (49%) | 275 | (47%) |
| DISORDERS | | | | | | | | | | | | | |
| Akathisia | n (%) | 1 | (<1%) | 2 | (2%) | 3 | (3%) | 0 | | 6 | (5%) | 12 | (2%) |
| Balance Impaired Nos | n (%) | 0 | | 0 | | 0 | | 1 | (<1%) | 0 | | 1 | (<1%) |

Note:
Percentages for gender-specific adverse events are based on he number of subjects in the appropriate gender. Percentages for all other adverse events are based on the total number of subjects in the Safety population.
Note:
Each subject is counted at most once within a system organ class and preferred term. Adverse events were coded to system organ class and preferred term using the MedDRA dictionary, Version 5.0.
Note:
Treatment-emergent adverse events are defined as any adverse event that occurred following initiation of study medication or any pre-existing medical condition that worsened after randomization. Treatment-emergent adverse events include all adverse events reported through a subject's study discontinuation visit and all SAEs reported within 30 days after permanent discontinuation of study medication.

TABLE 39
Overall Incidence of Treatment-Emergent Adverse Events: Safety
Population.

| | | Treatment Group | | | | | | | | | | | |
|---|---|---|---|---|---|---|---|---|---|---|---|---|---|
| System Organ Class/ | | | | BX | | BX | | | | Risperidone | | | |
| Preferred Term | Statistic | BX 5 mg | | 10 mg | | 20 mg | | Placebo | | 6 mg | | Overall | |
| NERVOUS SYSTEM | | | | | | | | | | | | | |
| DISORDERS (cont'd) | | | | | | | | | | | | | |
| Disturbance in Attention | n (%) | 0 | | 0 | | 1 | (<1%) | 0 | | 1 | (<1%) | 2 | (<1%) |
| Dizziness | n (%) | 16 | (14%) | 12 | (10%) | 8 | (7%) | 12 | (10%) | 6 | (5%) | 54 | (9%) |
| Drooling | n (%) | 0 | | 0 | | 0 | | 1 | (<1%) | 2 | (2%) | 3 | (<1%) |
| Dysarthria | n (%) | 0 | | 0 | | 1 | (<1%) | 0 | | 2 | (2%) | 3 | (<1%) |
| Dyskinesia | n (%) | 1 | (<1%) | 1 | (<1%) | 0 | | 0 | | 0 | | 2 | (<1%) |
| Dystonia | n (%) | 0 | | 0 | | 1 | (<1%) | 1 | (<1%) | 5 | (4%) | 7 | (1%) |
| Extrapyramidal Disorder | n (%) | 1 | (1%) | 0 | | 2 | (2%) | 3 | (3%) | 4 | (3%) | 10 | (2%) |
| Gait Abnormal Nos | n (%) | 1 | (<1%) | 2 | (2%) | 0 | | 0 | | 0 | | 3 | (<1%) |
| Gait Feastinating | n (%) | 0 | | 0 | | 0 | | 0 | | 1 | (<1%) | 1 | (<1%) |
| Grand Mal Convulsion | n (%) | 0 | | 0 | | 0 | | 1 | (<1%) | 0 | | 1 | (<1%) |
| Headache Nos | n (%) | 34 | (30%) | 42 | (35%) | 29 | (25%) | 25 | (21%) | 33 | (28%) | 163 | (28%) |
| Hyperactivity Syndrome | n (%) | 0 | | 1 | (<1%) | 0 | | 0 | | 0 | | 1 | (<1%) |
| Aggravated | | | | | | | | | | | | | |
| Hypoaesthesia | n (%) | 0 | | 0 | | 1 | (<1%) | 0 | | 1 | (<1%) | 2 | (<1%) |
| Loss of Propriception | n (%) | 0 | | 1 | (<1%) | 0 | | 0 | | 0 | | 1 | (<1%) |
| Memory Impairment | n (%) | 0 | | 0 | | 3 | (3%) | 0 | | 0 | | 3 | (<1%) |
| Migraine Aggravated | n (%) | 0 | | 0 | | 0 | | 0 | | 1 | (<1%) | 1 | (<1%) |
| Migraine Nos | n (% | 0 | | 1 | (<1%) | 0 | | 0 | | 0 | | 1 | (<1%) |
| Oculogyric Crisis | n (%) | 0 | | 0 | | 1 | (<1%) | 0 | | 1 | (<1%) | 2 | (<1%) |
| Paraesthesia | n (%) | 0 | | 0 | | 0 | | 0 | | 1 | (<1%) | 1 | (<1%) |
| Parkinsonian Rest | n (%) | 1 | (<1%) | 0 | | 0 | | 0 | | 0 | | 1 | (<1%) |
| Tremor | | | | | | | | | | | | | |
| Parkinsonism | n (%) | 0 | | 0 | | 1 | (<1%) | 0 | | 0 | | 1 | (<1%) |

Note:
Percentages for gender-specific adverse events are based on the number of subjects in the appropriate gender. Percentages for all other adverse events are based on the total number of subjects in the Safety population.
Note:
Each subject is counted at most once within a system organ class and preferred term. Adverse events were coded to system organ class and preferred term using the MedDRA dictionary, Version 5.0.
Note:
Treatment-emergent adverse events are defined as any adverse event that occurred following initiation of study medication or any pre-existing medical condition that worsened after randomization. Treatment-emergent adverse events include all adverse events reported through a subject's study discontinuation visit and all SAEs reported within 30 days after permanent discontinuation of study medication.

TABLE 40
Overall Incidence of Treatment-Emergent Adverse Events: Safety
Population.

| | | Treatment Group | | | | | |
|---|---|---|---|---|---|---|---|
| System Organ Class/ | | BX | BX | BX | | Risperidone | |
| Preferred Term | Statistic | 5 mg | 10 mg | 20 mg | Placebo | 6 mg | Overall |

NERVOUS SYSTEM DISORDERS (cont'd)

| | | | | | | | | | | | | | |
|---|---|---|---|---|---|---|---|---|---|---|---|---|---|
| Sedation | n (%) | 6 | (5%) | 10 | (8%) | 10 | (9%) | 10 | (8%) | 15 | (13%) | 51 | (9%) |
| Somnolence | n (%) | 4 | (3%) | 2 | (2%) | 6 | (5%) | 5 | (4%) | 5 | (22%) | 22 | (4%) |
| Syncope | n (%) | 1 | (<1%) | 3 | (3%) | 2 | (2%) | 1 | (<1%) | 0 | | 7 | (1%) |
| Tardive Dyskinesia | n (%) | 0 | | 1 | (<1%) | 1 | (<1%) | 2 | (2%) | 0 | | 4 | (<1%) |
| Tremor | n (%) | 3 | (3%) | 0 | | 1 | (<1%) | 1 | (<1%) | 1 | (<1%) | 6 | (1%) |
| Trismus | n (%) | 1 | (<1%) | 0 | | 0 | | 0 | | 0 | | 1 | (<1%) |
| PSYCHIATRIC | n (%) | 47 | (41%) | 38 | (32%) | 46 | (40%) | 44 | (37%) | 47 | (39%) | 222 | (38%) |
| DISORDERS | | | | | | | | | | | | | |
| Acute Psychosis | n (%) | | (<1%) | 0 | | 0 | | 0 | | 0 | | 1 | (<1%) |
| Adjustment Disorder | n (%) | 0 | | 0 | | 1 | (<1%) | 0 | | 0 | | 1 | (<1%) |
| Nos | | | | | | | | | | | | | |
| Affect Lability | n (%) | 0 | | 0 | | 1 | (<1%) | 0 | | 1 | (<1%) | 2 | (<1%) |
| Aggression | n (%) | 1 | (<1%) | 0 | | 1 | (<1%) | 1 | (<1%) | 0 | | 3 | (<1%) |
| Agitation | n (%) | 13 | (11%) | 11 | (9%) | 13 | (11%) | 11 | (9%) | 9 | (8%) | 57 | (10%) |
| Agitation Aggravated | n (%) | 5 | (4%) | 5 | (4%) | 1 | (<1%) | 2 | (2%) | 0 | | 13 | (2%) |
| Alcoholic Hangover | n (%) | 0 | | 0 | | 0 | | 0 | | 1 | (<1%) | 1 | (<1%) |
| Anhedonia | n (%) | 0 | | 0 | | 0 | | 1 | (<1%) | 0 | | 1 | (<1%) |
| Anxiety | n (%) | 6 | (5%) | 12 | (10%) | 7 | (6%) | 6 | (5%) | 7 | (6%) | 38 | (6%) |
| Anxiety Aggravated | n (%) | 3 | (3%) | 3 | (3%) | 2 | (2%) | 2 | (2%) | 4 | (3%) | 14 | (2%) |
| Bruxism | n (%) | 0 | | 0 | | 1 | (<1%) | 0 | | 0 | | 1 | (<1%) |
| Catatonia | n (%) | 0 | | 0 | | 0 | | 1 | (<1%) | 0 | | 1 | (<1%) |
| Confusion | n (%) | 1 | (<1%) | 0 | | 1 | (<1%) | 0 | | 0 | | 2 | (<1%) |

Note:
Percentages for gender-specific adverse events are based on the number of subjects in the appropriate gender. Percentages for all other adverse events are based on the total number of subjects in the Safety population.
Note:
Each subject is counted at most once within a system organ class and preferred term. Adverse events were coded to system organ class and preferred term using the MedDRA dictionary, Version 5.0.
Note:
Treatment-emergent adverse events are defined as any adverse event that occurred following initiation of study medication or any pre-existing medical condition that worsened after randomization. Treatment-emergent adverse events include all adverse events reported through a subject's study discontinuation visit and all SAEs reported within 30 days after permanent discontinuation of study medication.

CLINICAL STUDY TWO: The following treatment-emergent adverse events were reported related to EPS:

TABLE 41
Overall Incidence of Treatment-Emergent Adverse Events: Safety
Population.

| | | Treatment Group | | | | | | | | | |
|---|---|---|---|---|---|---|---|---|---|---|---|
| System Organ Class/ | | | | | | | | Risperidone | | | |
| Preferred Term | Statistic | BX 30 mg | | BX 40 mg | | Placebo | | 6 mg | | Overall | |
| NERVOUS SYSTEM | n (%) | 52 | (36%) | 55 | (37%) | 40 | (27%) | 82 | (53%) | 229 | (38%) |
| DISORDERS | | | | | | | | | | | |
| AKATHISIA | n (%) | 5 | (3%) | 10 | (7%) | 5 | (3%) | 16 | (10%) | 36 | (6%) |
| BALANCE | n (%) | 1 | (<1%) | 0 | | 0 | | 0 | | 1 | (<1%) |
| DISORDER | | | | | | | | | | | |
| BRADYKINESIA | n (%) | 0 | | 0 | | 0 | | 2 | (1%) | 2 | (<1%) |
| CHOREOATHETOSIS | n (%) | 0 | | 1 | (<1%) | 0 | | 0 | | 1 | (<1%) |
| COGWHEEL | n (%) | 1 | (<1%) | 0 | | 0 | | 0 | | 1 | (<1%) |
| RIGIDITY | | | | | | | | | | | |
| DISTURBANCE IN | n (%) | 1 | (<1%) | 0 | | 0 | | 2 | (1%) | 3 | (<1%) |
| ATTENTION | | | | | | | | | | | |
| DIZZINESS | n (%) | 11 | (8%) | 20 | (14%) | 6 | (4%) | 9 | (6%) | 46 | (8%) |
| DYSARTHRIA | n (%) | 1 | (<1%) | 0 | | 0 | | 3 | (2%) | 4 | (<1%) |
| DYSGEUSIA | n (%) | 1 | (<1%) | 0 | | 0 | | 0 | | 1 | (<1%) |
| DYSKINESIA | n (%) | 2 | (1%) | 4 | (3%) | 0 | | 1 | (<1%) | 7 | (1%) |
| DYSTONIA | n (%) | 3 | (2%) | 3 | (2%) | 1 | (<1%) | 11 | (7%) | 18 | (3%) |
| EXTRAPYRAMIDAL | n (%) | 7 | (5%) | 8 | (5%) | 8 | (<5%) | 21 | (14%) | 44 | (7%) |
| DISORDER | | | | | | | | | | | |
| HEADACHE NOS | n (%) | 17 | (12%) | 25 | (17%) | 21 | (14%) | 23 | (15%) | 86 | (14%) |

*Denotes gender-specific adverse events.
Note:
Percentages for gender-specific adverse events are based in the number of subjects on the appropriate gender. Percentages for all other adverse events are based on the total number of subjects in the Safety Population.
Note:
Each subject is counted at most once within a system organ class and preferred term. Adverse events were coded to system organ class and preferred term using the MedDRA dictionary, Version 6.1.
Note:
Treatment-emergent adverse events are defined as any adverse event that occurred following initiation of study medication or any pre-existing medical condition that worsened in severity after randomization. Treatment-emergent adverse events include all adverse events reported through a subject's study discontinuation visit and all SAEs reported within 30 days after study discontinuation.
Note:
Adverse events include new or worsened physical examination abnormalities.

TABLE 42
Overall Incidence of Treatment-Emergent Adverse Events: Safety
Population.

| | | Treatment Group | | | | |
|---|---|---|---|---|---|---|
| System Organ Class/ | | | | | Risperidone | |
| Preferred Term | Statistic | BX 30 mg | BX 40 mg | Placebo | 6 mg | Overall |

NERVOUS SYSTEM DISORDERS (cont'd)

| | | | | | | |
|---|---|---|---|---|---|---|
| HYPOAESTHESIA | n (%) | 0 | 0 | 0 | 1 (<1%) | 1 (<1%) |
| MEMORY | n (%) | 0 | 0 | 1 (<1%) | 1 (<1%) | 2 (<1%) |
| IMPAIRMENT | | | | | | |
| MIGRAINE | n (%) | 1 (<1%) | 0 | 0 | 0 | 1 (<1%) |
| PARAESTHESIA | n (%) | 1 (<1%) | 0 | 0 | 0 | 1 (<1%) |
| PARKINSONISM | n (%) | 0 | 0 | 0 | 2 (1%) | 2 (<1%) |
| PSYCHOMOTOR | n (%) | 1 (<1%) | 0 | 0 | 0 | 1 (<1%) |
| HYPERACTIVITY | | | | | | |
| SEDATION | n (%) | 7 (5%) | 4 (3%) | 2 (1%) | 11 (7%) | 24 (4%) |
| SINUS HEADACHE | n (%) | 0 | 0 | 0 | 1 (<1%) | 1 (<1%) |
| SOMNOLENCE | n (%) | 6 | 1 (<1%) | 2 (<1%) | 9 (6%) | 18 (3%) |
| SYNCOPE | n (%) | 1 (<1%) | 0 | 0 | 0 | 1 (<1%) |
| TARDIVE | n (%) | 2 (<1%) | 1 (<1%) | 0 | 3 (2%) | 6 (1%) |
| DYSKINESIA | | | | | | |
| TREMOR | n (%) | 9 (6%) | 10 (7%) | 7 (5%) | 7 (5%) | 33 (6%) |

*Denotes gender-specific adverse events.
Note:
Percentages for gender-specific adverse events are based in the number of subjects on the appropriate gender. Percentages for all other adverse events are based on the total number of subjects in the Safety Population.
Note:
Each subject is counted at most once within a system organ class and preferred term. Adverse events were coded to system organ class and preferred term using the MedDRA dictionary, Version 6.1.
Note:
Treatment-emergent adverse events are defined as any adverse event that occurred following initiation of study medication or any pre-existing medical condition that worsened in severity after randomization. Treatment-emergent adverse events include all adverse events reported through a subject's study discontinuation visit and all SAEs reported within 30 days after study discontinuation.
Note:
Adverse events include new or worsened physical examination abnormalities.

CLINICAL STUDY THREE: The following treatment-emergent adverse-events were reported relating to EPS:

TABLE 43
Overall Incidence of Treatment-Emergent Adverse Events: Safety
Population.

| | | Treatment Group | | | | |
|---|---|---|---|---|---|---|
| | | | | | Risperidone | |
| System Organ Class/ | | BX 20 mg | BX 40 mg | Placebo | 6 mg | Overall |
| Preferred Term | Statistic | (N = 154) | (N = 150) | (N = 149) | (N = 150) | (N = 603) |

MUSCULOSKELETAL AND CONNECTIVE TISSUE
DISORDERS (cont'd)

| | | | | | | |
|---|---|---|---|---|---|---|
| MUSCULOSKELETAL | n (%) | 0 | 1 (<1%) | 0 | 1 (<1%) | 2 (<1%) |
| STIFFNESS | | | | | | |
| MYALGIA | n (%) | 1 (<1%) | 2 (1%) | 0 | 0 | 3 (<1%) |
| NECK PAIN | n (%) | 2 (1%) | 0 | 0 | 2 (1%) | 4 (<1%) |
| PAIN IN EXTREMITY | n (%) | 1 (<1%) | 0 | 4 (3%) | 3 (2%) | 8 (1%) |
| PAIN IN JAW | n (%) | 0 | 1 (<1%) | 0 | 0 | 1 (<1%) |
| NEOPLASMS BENIGN, | n (%) | 0 | 1 (<1%) | 0 | 0 | 1 (<1%) |
| MALIGNANT AND | | | | | | |
| UNSPECIFIED (INCL CYSTS | | | | | | |
| AND POLYPS) | | | | | | |
| CYST | n (%) | 0 | 1 (<1%) | 0 | 0 | 1 (<1%) |
| NERVOUS SYSTEM | n (%) | 58 (38%) | 55 (37%) | 59 (40%) | 56 (37%) | 228 (38%) |
| DISORDERS | | | | | | |
| AKATHISIA | n (%) | 5 (3%) | 4 (3%) | 2 (1%) | 4 (3%) | 15 (2%) |
| BALANCE DISORDER | n (%) | 0 | 0 | 0 | 1 (<1%) | 1 (<1%) |
| BURNING SENSATION | n (%) | 0 | 0 | 2 (1%) | 0 | 2 (<1%) |

*Denotes gender-specific adverse events.
Note:
Percentages for gender-specific adverse events are based in the number of subjects on the appropriate gender. Percentages for all other adverse events are based on the total number of subjects in the Safety Population.
Note:
Each subject is counted at most once within a system organ class and preferred term. Adverse events were coded to system organ class and preferred term using the MedDRA dictionary, Version 6.1.
Note:
Treatment-emergent adverse events are defined as any adverse event that occurred following initiation of study medication or any pre-existing medical condition that worsened in severity after randomization. Treatment-emergent adverse events include all adverse events reported through a subject's study discontinuation visit and all SAEs reported within 30 days after study discontinuation.
Note:
Adverse events include new or worsened physical examination abnormalities.

TABLE 44
Overall Incidence of Treatment-Emergent Adverse Events: Safety
Population.

| | | Treatment Group | | | | |
|---|---|---|---|---|---|---|
| | | | | | Risperidone | |
| System Organ Class/ | | BX 20 mg | BX 40 mg | Placebo | 6 mg | Overall |
| Preferred Term | Statistic | (N = 154) | (N = 150) | (N = 149) | (N = 150) | (N = 603) |

NERVOUS SYSTEM DISORDERS (cont'd)

| | | | | | | |
|---|---|---|---|---|---|---|
| CONVULSION | n (%) | 2 (1%) | 0 | 0 | 0 | 2 (<1%) |
| DISTURBANCE IN | n (%) | 0 | 0 | 1 (<1%) | 0 | 1 (<1%) |
| ATTENTION | | | | | | |
| DIZZINESS | n (%) | 13 (8%) | 15 (10%) | 9 (6%) | 8 (5%) | 45 (7%) |
| DYSARTHRIA | n (%) | 0 | 0 | 1 (<1%) | 1 (<1%) | 2 (1%) |
| DYSKINESIA | n (%) | 2 (1%) | 2 (1%) | 2 (1%) | 1 (<1%) | 7 (1%) |
| DYSTONIA | n (%) | 2 | 0 | 2 (1%) | 0 | 4 (1%) |
| EXTRAPYRAMIDAL | n (%) | 5 (3%) | 1 (<1%) | 4 (3%) | 2 (1%) | 12 (2%) |
| DISORDER | | | | | | |
| FACIAL PALSY | n (%) | 0 | 0 | 1 (<1%) | 0 | 1 (<1%) |
| HEADACHE | n (%) | 30 (19%) | 29 (19%) | 32 (21%) | 18 (12%) | 109 (18%) |
| HYPOAESTHESIA | n (%) | 0 | 0 | 1 (<1%) | 1 (<1%)) | 2 (<1%) |
| HYPOKINESIA | n (%) | 0 | 0 | 0 | 1 (<1%) | 1 (<1%) |
| LOSS OF | n (%) | 1 (<1%) | 0 | 0 | 0 | 1 (<1%) |
| CONSCIOUSNESS | | | | | | |
| MENTAL IMPAIRMENT | n (%) | 0 | 0 | 1 (<1%) | 0 | 1 (<1%) |

*Denotes gender-specific adverse events.
Note:
Percentages for gender-specific adverse events are based in the number of subjects on the appropriate gender. Percentages for all other adverse events are based on the total number of subjects in the Safety Population.
Note:
Each subject is counted at most once within a system organ class and preferred term. Adverse events were coded to system organ class and preferred term using the MedDRA dictionary, Version 6.1.
Note:
Treatment-emergent adverse events are defined as any adverse event that occurred following initiation of study medication or any pre-existing medical condition that worsened in severity after randomization. Treatment-emergent adverse events include all adverse events reported through a subject's study discontinuation visit and all SAEs reported within 30 days after study discontinuation.
Note:
Adverse events include new or worsened physical examination abnormalities.

TABLE 45
Overall Incidence of Treatment-Emergant adverse Events: Safety
Population.

| | | Treatment Group | | | | |
|---|---|---|---|---|---|---|
| | | | | | Risperidone | |
| System Organ Class/ | | BX 20 mg | BX 40 mg | Placebo | 6 mg | Overall |
| Preferred Term | Statistic | (N = 154) | (N = 150) | (N = 149) | (N = 150) | (N = 603) |

NERVOUS SYSTEM DISORDERS (cont'd)

| | | | | | | |
|---|---|---|---|---|---|---|
| MIGRAINE | n (%) | 0 | 0 | 1 (<1%) | 0 | 1 (<1%) |
| NEUROLOGICAL | n (%) | 0 | 1 (<1%) | 0 | 0 | 1 (<1%) |
| SYMPTOM | | | | | | |
| PARAESTHESIA | n (%) | 0 | 2 (<1%) | 1 (<1%) | 1 (<1%) | 4 (<1%) |
| RESTLESS LEGS | n (%) | 0 | 0 | 0 | 1 (<1%) | 1 (<1%) |
| SYNDROME | | | | | | |
| SEDATION | n (%) | 8 (5%) | 10 (7%) | 6 (4%) | 12 (8%) | 36 (6%) |
| SINUS HEADACHE | n (%) | 1 (<1%) | 0 | 0 | 0 | 1 (<1%) |
| SOMNOLENCE | n (%) | 5 (3%) | 4 (3%) | 7 (5%) | 13 (9%) | 29 (5%) |
| SPEECH DISORDER | n (%) | 0 | 0 | 0 | 1 (<1%) | 1 (<1%) |
| SYNCOPE VASOVAGAL | n (%) | 0 | 2 (1%) | 0 | 0 | 2 (<1%) |
| TARDIVE DYSKINESIA | n (%) | 1 (<1%) | 0 | 0 | 0 | 1 (<1%) |
| TREMOR | n (%) | 2 (1%) | 4 (3%) | 5 (3%) | 4 (3%) | 15 (2%) |

*Denotes gender-specific adverse events.
Note:
Percentages for gender-specific adverse events are based in the number of subjects on the appropriate gender. Percentages for all other adverse events are based on the total number of subjects in the Safety Population.
Note:
Each subject is counted at most once within a system organ class and preferred term. Adverse events were coded to system organ class and preferred term using the MedDRA dictionary, Version 6.1.
Note:
Treatment-emergent adverse events are defined as any adverse event that occurred following initiation of study medication or any pre-existing medical condition that worsened in severity after randomization. Treatment-emergent adverse events include all adverse events reported through a subject's study discontinuation visit and all SAEs reported within 30 days after study discontinuation.
Note:
Adverse events include new or worsened physical examination abnormalities.

Clinical Study Four:

The proportion of patients with TEAEs related to EPS was lower in the PBO group (4%) than in either of the BX groups (BX20: 10%; BX30 15%), as provided in Table 46. TEAEs related to EPS for which there were ≧3 patients in either BX group relative to the PBO group comprised: BX20—akathisia; BX30—dyskinesia, akathisia, extrapyramidal disorder. In total, 15 patients had TEAEs related to EPS that led to withdrawal (2 in the PBO group; 4 in the BX20 group; 9 in the BX30 group).

TABLE 46
All EPS TEAEs by SOC and Preferred Term (APTS).

| System Organ Class | PBO | | BX20 | | BX30 | |
|---|---|---|---|---|---|---|
| & Preferred Term | n | (%) | n | (%) | n | (%) |
| Patients Treated | 166 | | 159 | | 172 | |
| Patients with EPS | 7 | (4.2) | 16 | (10.1) | 25 | (14.5) |
| TEAEs | | | | | | |
| NERVOUS SYSTEM | 7 | (4.2) | 16 | (10.1) | 25 | (14.5) |
| DISORDERS | | | | | | |
| Dyskinesia | 1 | (0.6) | 2 | (1.3) | 11 | (6.4) |
| Akathisia | 2 | (1.2) | 11 | (6.9) | 8 | (4.7) |
| Extrapyramidal disorder | | | 1 | (0.6) | 5 | (2.9) |
| Tremor | 2 | (1.2) | 1 | (0.6) | 5 | (2.9) |
| Athetosis | | | | | 1 | (0.6) |
| Choreoathetosis | 2 | (1.2) | 3 | (1.9) | 1 | (0.6) |
| Dystonia | | | 1 | (0.6) | | |
| MUSCULOSKELETAL | | | 1 | (0.6) | 1 | (0.6) |
| AND | | | | | | |
| CONNECTIVE TISSUE | | | | | | |
| DISORDERS | | | | | | |
| Muscle rigidity | | | 1 | (0.6) | 1 | (0.6) |

(GS) Refers to a Gender Specific AE
Coding is done in MedDRA version 8.1

### SAS, BAS & AIMS

CLINICAL STUDY ONE: Simpson-Angus Scale global scores were evaluated as normal or abnormal at Baseline and Endpoint. The percentages of subjects with normal and abnormal scores at each time point. At Baseline, the percentages of subjects with abnormal SAS scores ranged from 8% to 12%. At Endpoint, the percentages of subjects with abnormal SAS scores ranged from 3% to 10%. The percentage of subjects with abnormal SAS scores decreased between Baseline and Endpoint in all treatment groups. There were no statistically significant differences among the treatment groups in the percentage of subjects shifting between categories from Baseline to Endpoint (p=0.800).

The BAS score consists of an objective score, a score awareness of restlessness, a score of distress related to restlessness, a 3-item total score, and a global clinical assessment score. There were no statistically significant differences among the treatment groups at Baseline or Endpoint for the objective score, awareness of restlessness score, distress related to restlessness score, the 3-item total score, or the global clinical assessment score.

There were no statistically significant differences among the treatment groups in the percentage of subjects shifting between categories from Baseline to Endpoint in BAS total scores or BAS Global Clinical Assessment scores. There were no statistically significant differences among the treatment groups at Baseline or Endpoint. There was no statistically significant difference among the treatment groups in the values for change from Baseline to Endpoint.

CLINICAL STUDY TWO: Simpson-Angus Scale global scores were evaluated as normal or abnormal at Baseline and Endpoint. At Baseline, the percentages of subjects with abnormal SAS scores were: 14% in the 30 mg bifeprunox group, 11% in the 40 mg bifeprunox group, 7% in the placebo group, and 6% in the risperidone group. The percentage of subjects with abnormal SAS scores between Baseline and Endpoint decreased in the bifeprunox groups, were unchanged in the placebo group, and increased in the risperidone group. At Endpoint, the percentages of subjects with abnormal SAS scores ranged from 7% in the 40 mg bifeprunox and placebo groups to 14% in the risperidone group.

Shifts from normal at Baseline to abnormal at Endpoint were the highest in the risperidone group (19 subjects, 12%) compared to the other three treatment groups (30 mg bifeprunox: one subject, <1%; 40 mg bifeprunox: five subjects, 4%; placebo (six subjects, 4%). A statistically significant difference (p<0.001) among the treatment groups was observed.

The BAS score consists of an objective score, a subjective awareness of restlessness score, a score of distress related to restlessness, a 3-item total score, and a global clinical assessment score. There were no statistically significant differences across the treatment groups at Endpoint for the objective score (p=0.093), awareness of restlessness score (p=0.368), distress related to restlessness score (p=0.779), the 3-item total BAS score (p=0.433), or the global clinical assessment of akathisia score (p=0.541). There were also no statistically significant differences across treatment groups at Baseline for the above measures (p≧0.168).

There were no statistically significant differences across the treatment groups in the percentage of subjects shifting between categories from Baseline to Endpoint in BAS total scores (p=0.482) or BAS Global Clinical Assessment scores (p=0.911).

There were no statistically significant differences across the treatment groups at Baseline (mean range=1.0 to 1.2; p=0.923) or Endpoint (mean range=0.9 to 1.6; p=0.138). There was no statistically significant difference across the treatment groups in the values for change from Baseline to Endpoint (mean range=−0.3 to 0.4; p=0.138).

CLINICAL STUDY THREE: Simpson-Angus Scale global scores were evaluated as normal or abnormal at Baseline and Endpoint. At Baseline, the percentages of subjects with abnormal SAS scores ranged from 6% to 10%. At Endpoint, the percentages of subjects with abnormal SAS scores ranged from 3% to 8%. The percentage of subjects with abnormal SAS scores decreased between

Baseline and Endpoint in all treatment groups. There were no statistically significant differences among the treatment groups in the percentage of subjects shifting between categories from Baseline to Endpoint (p=0.463). The BAS score consists of an objective score, a subjective awareness of restlessness score, a score of distress related to restlessness, a 3-item total score, and a global clinical assessment score. There were no statistically significant differences among the treatment groups at Endpoint for the objective score (p=0.421), awareness of restlessness score (p=0.584), distress related to restlessness score (p=0.254), the 3-item total score (p=0.865), or the global clinical assessment score (p=0.518). There was a statistically significant difference among treatment groups at Baseline for the subjective distress related to restlessness score (p=0.029), but not for any other scores.

There were no statistically significant differences among the treatment groups in the percentage of subjects shifting between categories from Baseline to Endpoint in BAS total scores (p=0.808) or BAS Global Clinical Assessment scores (p=0.525).

There were no statistically significant differences among the treatment groups at Baseline (p=0.362) or Endpoint (p=0.187). There was no statistically significant difference among the treatment groups in the values for change from Baseline to Endpoint (p=0.187).

Clinical Study Four:

SAS

At baseline, the mean SAS total scores in all three treatment groups ranged from 2.17 to 2.33.

There were minor fluctuations in the mean SAS total scores in all three treatment groups during the study, and at Month 6, the mean SAS total scores ranged from 0.57 to 1.16 (APTS, OC); thus, the mean total scores at baseline and at Month 6 were within normal range. The adjusted mean maximal changes from baseline to Month 6 in SAS total scores were <1 in each treatment group (APTS, OC, ANCOVA). There were no clinically relevant differences between any of the treatment groups in SAS total scores.

At baseline, the majority of patients (PBO: 74%; BX20: 71%; BX30: 72%) were normal with respect to SAS status and the proportion of normal patients had increased at Month 6 (PBO: 85%; BX20: 89%; BX30: 78%). There were no statistically significant differences between any of the treatment groups in the shifts in SAS status (categories: no change; abnormal to normal; normal to abnormal).

BAS

At baseline, the mean BAS total scores in all three treatment groups ranged and from 0.44 to 0.46. There were minor fluctuations in the mean BAS total score in all three treatment groups during the study, and at Month 6, the mean BAS total scores ranged from 0.05 to 0.17 (APTS, OC). The adjusted mean maximal changes from baseline to Month 6 in BAS total scores were <0.6 in each treatment group (APTS, OC, ANCOVA). There were no clinically relevant differences between any of the treatment groups in BAS total scores.

For the BAS global assessment scores, the trends were similar: there were minor fluctuations in the mean BAS global assessment scores in all three treatment groups during the study; the mean scores ranged from 0.22 to 0.26 at baseline, and from 0.07 to 0.14 at Month 6 (APTS, OC). There were no clinically relevant differences between any of the treatment groups in BAS global assessment scores at Month 6 (APTS, OC). There were no statistically significant differences between any of the treatment groups in the distribution of BAS items 1, 2, or 3.

AIMS

From Table 47 below, the mean AIMS total scores in all three treatment groups were low and ranged from 1.04 to 1.35 at baseline. There was for all treatment groups an initial increase in the adjusted mean scores (largest in the BX30 group), after which the mean scores decreased over time; at Month 6, the scores had returned to baseline level in all three treatment groups (PBO: 0.11; BX20: 1.08; BX30: 0.86 (APTS, OC)). The adjusted mean changes in AIMS total scores in the BX20 (all time points) and BX30 (Months 5 and 6) groups were not statistically significantly different from those in the PBO group, whereas the adjusted mean change for BX30 (from Week 1 to Month 4) group was. The adjusted mean maximal changes from baseline to Month 6 in AIMS total score were small (PBO: 0.39; BX20: 1.21; BX30: 2.45, APTS, OC, ANCOVA). None of the differences were considered clinically significant.

TABLE 47
Movement Rating Scales Scores (APTS).

| | PBO^{a} | | BX20^{b} | | BX30^{c} | |
|---|---|---|---|---|---|---|
| Scale | OC | LOCF | OC | LOCF | OC | LOCF |

SAS

| | | | | | | |
|---|---|---|---|---|---|---|
| Baseline | 2.17 (3.54) | 2.17 (3.54) | 2.27 (3.72) | 2.27 (3.72) | 2.33 (3.53) | 2.33 (3.53) |
| Week 6 | 1.42 (2.44) | 1.54 (2.79) | 1.31 (2.69) | 1.43 (2.77) | 2.06 (3.41) | 2.35 (3.87) |
| Month 6 | 0.57 1.26) | 1.33 (2.68) | 0.97 (2.20) | 1.22 (2.47) | 1.16 (2.78) | 2.09 (3.64) |

BAS

| | | | | | | |
|---|---|---|---|---|---|---|
| Baseline | 0.46 (1.13) | 0.46 (1.13) | 0.46 (1.10) | 0.44 (1.10) | 0.45 (I.11) | 0.45 (1.11) |
| Week 6 | 0.30 (0.76) | 0.34 (0.94) | 0.36 (0.99) | 0.49 (1.21) | 0.35 (I.01) | 0.66 (1.55) |
| Month 6 | 0.05 (0.21) | 0.34 (0.99) | 0.17 (0.42) | 0.49 (1.25) | 0.16 (0.65) | 0.69 (I.58) |

AIMS

| | | | | | | |
|---|---|---|---|---|---|---|
| Baseline | 1.10 (2.82) | 1.10 (2.82) | 1.04 (2.76) | 1.04 (2.76) | 1.35 (2.79) | 1.35 (2.79) |
| Week 6 | 0.92 (2.70) | 1.05 (3.12) | 1.16 (2.55) | 1.59 (3.96) | 1.80 (4.25) | 2.83 (6.09) |
| Month 6 | 0.11 (0.44) | 0.86 (3.03) | 1.08 (2.13) | 1.55 (3.92) | 0.86 (3.18) | 2.77 (6.09) |

Values are means (SD).
^{a}PBO - OC: baseline: n = 166, Week 6: n = 112, Month 6: n = 44; LOCF: n = 166
^{b}BX20 - OC: baseline: n = 159, Week 6: n = 104, Month 6: n = 59; LOCF: n = 159
^{c}BX30 - OC: baseline: n = 172, Week 6: n = 106, Month 6: n = 57; LOCF: n = 172

### Body Weight

Clinical Study One:

Small decreases in weight were observed in the bifeprunox treatment groups, but not in the placebo treatment group. At Endpoint, the mean weight loss for subjects in the bifeprunox treatment groups was approximately 1 lb (5 mg bifeprunox, −1.0 lb; 10 mg bifeprunox, −1.3 lb; 20 mg bifeprunox, −0.6 lbs). The placebo treatment group had a mean weight gain of 1.9 lbs.

Statistical testing of the change from Baseline in weight was performed using similar methodology to that used for the secondary efficacy parameters (an ANCOVA model with factors for treatment and weight at Baseline). Pairwise comparisons of the bifeprunox treatment groups versus placebo were all statistically significant (bifeprunox 5 mg [p=0.025], bifeprunox 10 mg [p=0.009] and bifeprunox 20 mg [p=0.031] for the difference between bifeprunox and placebo mean change from Baseline in weight at Endpoint.

The mean weight change of subjects in the risperidone treatment group was a 4.8 lb increase.

Increases in weight: The percentages of subjects whose weight increased by more than 7% in the bifeprunox treatment groups (2 to 4%) were less than or similar to the percentage observed in the placebo treatment group (5%). In the risperidone treatment group, 13% of subjects increased their weight by ≧7%.

Decreases in weight: The percentage of subjects whose weight decreased by more than 7% was higher in the bifeprunox treatment groups (5 mg, 6%, 10 mg, 7%; 20 mg, 8%) than in the placebo treatment group (3%). No subjects in the risperidone treatment group decreased their weight by ≧7%.

Clinical Study Two:

Small decreases in mean body weight were observed in the bifeprunox treatment groups and the placebo group at Endpoint, while an increase was noted for the risperidone treatment group (Table 48 below). At Endpoint, the mean weight changes were: −2.2 lbs in 30 mg bifeprunox group; −19 lbs in 40 mg bifeprunox group; 0.5 lbs in placebo group; and 3.2 lbs in risperidone group. In the bifeprunox treatment groups, mean changes at Week 6 were higher than those at Endpoint.

TABLE 48
Changes in Body Weight at Week 6 and Endpoint: Safety
Population.

| | Treatment Group | | | |
|---|---|---|---|---|
| | Bifeprunox | Bifeprunox | | Risperidone |
| | 30 mg | 40 mg | Placebo | 6 mg |
| Total Number of Subjects | N = 145 | N = 147 | N = 149 | N = 154 |
| Weight (lbs) | | | | |
| Baseline, n | 145 | 147 | 149 | 154 |
| Mean (SD) | 169.2 (55.3) | 161.9 (46.6) | 171.4 (54.1) | 174.3 (58.5) |
| Median | 161 | 155 | 165 | 170 |
| Min, Max | 84, 360 | 84, 378 | 88, 353 | 82, 404 |
| Change from Baseline at Week | 68 | 48 | 48 | 84 |
| 6, n | | | | |
| Baseline Mean | 166.1 | 162.0 | 172.6 | 166.6 |
| Mean (SD) | −4.3 (8.2) | −3.1 (5.8) | −0.6 (7.1) | 4.1 (7.6) |
| Median | −2 | −2 | 0 | 2 |
| Min, Max | −32, 8 | 18, 10 | −17, 16 | −12, 33 |
| Change from Baseline at | 138 | 134 | 140 | 150 |
| Endpoint, n | | | | |
| Baseline Mean | 168.1 | 160.7 | 170.8 | 174.5 |
| Mean (SD) | −2.2 (6.7) | −1.9 (5.8) | 0.5 (6.0) | 3.2 (7.4) |
| Median | 0 | −1 | 0 | 2 |
| Min, Max | −32, 9 | −18, 15 | −17, 20 | −20, 33 |

Note:
Endpoint was defined as the last scheduled (including early termination), non-missing, post-Baseline evaluation (excluding the follow-up visit) collected during the study.

CLINICAL STUDY THREE: Small decreases in mean body weight were observed in the bifeprunox treatment groups and the placebo group, while an increase was noted for the olanzapine treatment group. At Endpoint, the mean weight changes were: −2.3 lbs in 20 mg bifeprunox group; −1.1 lbs in 30 mg bifeprunox group; −1.3 lbs in placebo group; and 5.2 lbs in olanzapine group. Mean changes at Week 6 were comparable to those at Endpoint (Table 49 below).

The incidence of markedly abnormal (≧7%) decrease in body weight was comparable among bifeprunox treatment and placebo groups (20 mg bifeprunox: 6%; 30 mg bifeprunox: 5%; placebo: 5%) and was lower in the olanzapine treatment group (<1%). The converse was seen in the incidence of markedly abnormal (≧7%) increase in body weight with 19% in the olanzapine treatment group compared to 5% in the 30 mg bifeprunox, 1% in the 20 mg bifeprunox, and 5% in the placebo group who experienced this.

TABLE 49
Change in Body Weight at Week 6 and Endpoint: Safety Population.

| | Treatment Group | | | |
|---|---|---|---|---|
| | Bifeprunox | Bifeprunox | | Olanzapine |
| | 20 mg | 30 mg | Placebo | 15 mg |
| Total Number of Subjects | N = 154 | N = 150 | N = 149 | N = 150 |
| Weight (lbs) | | | | |
| Baseline, n | 154 | 150 | 149 | 150 |
| Mean (SD) | 180.6 (61.7) | 179.1 (62.4) | 174.3 (55.3) | 175.2 (52.7) |
| Median | 176 | 171 | 169 | 173 |
| Min, Max | 77, 393 | 77, 358 | 75, 350 | 68, 300 |
| Change from Baseline at Week | 74 | 67 | 57 | 92 |
| 6, n | | | | |
| Baseline Mean | 171.9 | 172.6 | 173.9 | 173.1 |
| Mean (SD) | −3.3 (8.2) | −1.0 (8.9) | −1.6 (10.8) | 5.9 (7.3) |
| Median | −1 | −2 | 0 | 5 |
| Min, Max | −34, 26 | −18, 39 | −59, 17 | −7, 31 |
| Change from Baseline at | 142 | 138 | 143 | 141 |
| Endpoint, n | | | | |
| Baseline Mean | 178.8 | 179.7 | 175.2 | 173.7 |
| Mean (SD) | −2.3 (7.3) | −1.1 (7.4) | −1.3 (8.3) | 5.2 (6.9) |
| Median | −1 | 0 | 0 | 4 |
| Min, Max | −34, 26 | −22, 39 | −59, 41 | −7, 31 |

Note:
Endpoint was defined as the last scheduled (including early termination), non-missing, post-baseline evaluation (excluding the follow-up visit) collected during the study.

Clinical Study Four:

Weight and BMI and changes therein relative to baseline are summarized in the Tables 50 and 51 below. In all groups, the adjusted mean weight and BMI decreased from baseline to Month 6. The adjusted mean weight change from baseline to Month 6 (APTS, OC, ANCOVA) was −0.8 kg in the PBO group, −0.3 kg in the BX20 group, and −0.5 kg in the BX30 group. The adjusted mean weight decreases in the BX20 and the BX30 group were not statistically significantly different from that in the PBO group.

Weight decreased was reported as a TEAE for patients in all treatment groups (PBO: 5%; BX20: 8%; BX30: 8%). Weight increased was reported as a TEAE for patients in the PBO group (1.8%) and in the BX30 group (1.2%).

The adjusted mean weight change from baseline to Month 6 (APTS, LOCF, ANCOVA) in patients with/without nausea and/or vomiting showed that patients in all groups lost weight irrespective of whether they had nausea and/or vomiting, although those patients who also had nausea and/or vomiting had a greater weight decrease (PBO: −0.6 versus −1.9 kg; BX20: −1.0 versus −1.9 kg; BX30: −1.1 versus −2.3 kg.

TABLE 50
Weight, BMI and Waist Measurement (APTS)

| | | | | | | | | | PCS | | | |
|---|---|---|---|---|---|---|---|---|---|---|---|---|
| | | | | | | | | | Low | | High | |
| Assessment | Treatment | Visit | n | Mean | SD | Median | Min | Max | n | (%) | n | (%) |
| BMI | PBO | Baseline | 166 | 25.2 | 4.8 | 24.2 | 17.4 | 41.5 | | | | |
| | | Week 6 | 112 | 25.0 | 5.0 | 23.9 | 17.2 | 41.1 | | | | |
| | | Month 3 | 78 | 25.1 | 5.3 | 23.8 | 17.7 | 42.1 | | | | |
| | | Month 6 | 43 | 25.3 | 5.1 | 24.2 | 18.1 | 42.1 | | | | |
| | | Last* | 150 | 25.1 | 5.0 | 24.2 | 16 | 42.1 | | | | |
| | BX20 | Baseline | 159 | 24.7 | 4.3 | 24.0 | 17.3 | 40.9 | | | | |
| | | Week 6 | 104 | 24.3 | 4.2 | 23.8 | 16.7 | 39.3 | | | | |
| | | Month 3 | 81 | 23.9 | 4.2 | 23.1 | 16.3 | 40.2 | | | | |
| | | Month 6 | 59 | 23.9 | 3.7 | 23.1 | 15.9 | 32 | | | | |
| | | Last* | 143 | 24.3 | 4.2 | 23.7 | 15.9 | 40.2 | | | | |
| | BX30 | Baseline | 172 | 24.8 | 4.5 | 23.9 | 14.3 | 43.6 | | | | |
| | | Week 6 | 106 | 24.8 | 4.3 | 23.9 | 13.9 | 42.6 | | | | |
| | | Month 3 | 88 | 24.4 | 4.3 | 23.4 | 13.2 | 41.5 | | | | |
| | | Month 6 | 55 | 24.4 | 3.3 | 23.7 | 17.6 | 31.2 | | | | |
| | | Last* | 148 | 24.5 | 4.4 | 23.7 | 13.2 | 41.5 | | | | |

PCS: LOW: N/A HIGH: N/A

| | | | | | | | | |
|---|---|---|---|---|---|---|---|---|
| WAIST | PBO | Baseline | 90 | 84.0 | 12.8 | 82.0 | 62 | 117 |
| | | Week 6 | 62 | 83.8 | 12.4 | 82.0 | 62 | 117 |
| | | Month 3 | 47 | 84.3 | 12.0 | 82.8 | 62 | 115 |
| | | Month 6 | 31 | 85.1 | 12.4 | 85.0 | 67 | 116 |
| | | Last* | 91 | 84.70 | 12.8 | 83.9 | 62 | 116 |
| | BX20 | Baseline | 84 | 82.2 | 12.6 | 80.0 | 58 | 123 |
| | | Week 6 | 55 | 81.6 | 14.5 | 78.0 | 58 | 123 |
| | | Month 3 | 54 | 79.5 | 13.2 | 76.5 | 57.8 | 123 |
| | | Month 6 | 44 | 79.4 | 12.1 | 77.7 | 58.5 | 110 |
| | | Last* | 89 | 81.6 | 12.4 | 79.5 | 58.5 | 123 |
| | BX30 | Baseline | 93 | 83.7 | 15.8 | 81.0 | 55 | 160 |
| | | Week 6 | 59 | 83.9 | 16.7 | 80.0 | 62 | 160 |
| | | Month 3 | 54 | 81.6 | 14.8 | 79.5 | 61 | 154 |
| | | Month 6 | 38 | 81.1 | 10.9 | 78.0 | 62.5 | 112 |
| | | Last* | 94 | 82.6 | 15.3 | 80.4 | 61 | 158 |

PCS: LOW: N/A HIGH: N/A

| | | | | | | | | | | | | |
|---|---|---|---|---|---|---|---|---|---|---|---|---|
| WEIGHT | PBO | Baseline | 166 | 71.3 | 14.5 | 70.0 | 42 | 117 | | | | |
| | | Week 6 | 112 | 71.0 | 15.6 | 69.0 | 43 | 117 | 2 | (1.8) | 0 | (0.0) |
| | | Month 3 | 78 | 70.7 | 16.6 | 68.0 | 43 | 114 | 7 | (9.0) | 2 | (2.6) |
| | | Month 6 | 43 | 71.6 | 16.2 | 68.0 | 49 | 117 | 4 | (9.3) | 3 | (7.0) |
| | | Last* | 150 | 71.2 | 15.5 | 69.0 | 43 | 117 | 11 | (7.3) | 4 | (2.7) |
| | BX20 | Baseline | 159 | 70.7 | 13.9 | 69.0 | 45 | 125 | | | | |
| | | Week 6 | 104 | 69.7 | 13.8 | 67.0 | 46 | 123 | 5 | (4.8) | 0 | (0.0) |
| | | Month 3 | 81 | 68.1 | 14.1 | 66.0 | 45 | 126 | 8 | (9.9) | 0 | (0.0) |
| | | Month 6 | 59 | 68.2 | 12.3 | 67.0 | 45 | 99 | 6 | (10.2) | 4 | (6.8) |
| | | Last* | 143 | 69.7 | 13.5 | 68.0 | 45 | 126 | 13 | (9.1) | 4 | (2.8) |
| | BX30 | Baseline | 172 | 71.9 | 15.2 | 70.0 | 38 | 126 | | | | |
| | | Week 6 | 106 | 72.2 | 15.6 | 70.0 | 37 | 123 | 5 | (4.7) | 0 | (0.0) |
| | | Month 3 | 88 | 70.1 | 14.7 | 68.0 | 35 | 120 | 10 | (11.4) | 1 | (1.1) |
| | | Month 6 | 55 | 71.4 | 12.2 | 70.0 | 48 | 103 | 4 | (7.3) | 4 | (7.3) |
| | | Last* | 148 | 70.9 | 14.9 | 68.5 | 35 | 120 | 14 | (9.5) | 4 | (2.7) |

PCS: LOW: Decrease from baseline >= 7% HIGH: Increase from baseline % >= 7
*Last post-Baseline observation

TABLE 51
Change from Baseline in Weight, BMI and Waist Measurement
(APTS)

| | | | | | | | | | PCS | | | |
|---|---|---|---|---|---|---|---|---|---|---|---|---|
| | | | | | | | | | Low | | High | |
| Assessment | Treatment | Visit | n | Mean | SD | Median | Min | Max | n | (%) | n | (%) |
| BMI | PBO | Week 6 | 112 | −0.1 | 0.7 | 0.0 | −2.8 | 1.54 | | | | |
| | | Month 3 | 78 | −0.1 | 1.1 | 4.4 | −3.3 | 4.19 | | | | |
| | | Month 6 | 43 | −0.1 | 1.4 | 0.0 | −4.6 | 3.43 | | | | |
| | | Last* | 150 | −0.1 | 1.2 | 0.0 | −4.6 | 8.55 | | | | |
| | BX20 | Week 6 | 104 | −0.2 | 0.7 | 0.0 | −2.3 | 1.42 | | | | |
| | | Month 3 | 81 | −0.2 | 1.0 | 0.0 | −3.2 | 1.63 | | | | |
| | | Month 6 | 59 | −0.2 | 1.3 | 0.0 | −4.6 | 2.29 | | | | |
| | | Last* | 143 | −0.4 | 1.0 | 0.0 | −4.6 | 2.29 | | | | |
| | BX30 | Week 6 | 106 | −0.3 | 1.1 | 0.0 | −9.3 | 1.31 | | | | |
| | | Month 3 | 88 | −04 | 1.0 | −0.1 | −3.7 | 2.13 | | | | |
| | | Month 6 | 55 | −0.0 | 1.1 | 0.0 | −3 | 2.61 | | | | |
| | | Last* | 148 | −0.4 | 1.3 | 0.0 | −9.3 | 2.61 | | | | |

PCS: LOW: N/A HIGH: N/A

| | | | | | | | | |
|---|---|---|---|---|---|---|---|---|
| WAIST | PBO | Week 6 | 61 | 0.0 | 0.2 | 0.0 | −0.5 | 1 |
| | | Month 3 | 43 | 0.6 | 4.4 | 0.0 | −9 | 17.4 |
| | | Month 6 | 27 | −0.0 | 4.8 | 0.0 | −9.2 | 14.2 |
| | | Last* | 81 | −0.1 | 3.7 | 0.0 | −9.2 | 17.4 |
| | BX20 | Baseline | 55 | −0.0 | 0.3 | 0.0 | −2 | 0 |
| | | Week 6 | 44 | −1.1 | 3.9 | 0.0 | −21 | 3.2 |
| | | Month 3 | 35 | −1.1 | 3.8 | 0.0 | −12 | 4.4 |
| | | Month 6 | 75 | −0.9 | 3.3 | 0.0 | −15 | 4.4 |
| | BX30 | Week 6 | 59 | −0.0 | 0.3 | 0.0 | −2 | 0 |
| | | Month 3 | 47 | −0.4 | 2.5 | 0.0 | −10 | 4.5 |
| | | Month 6 | 30 | −0.0 | 4.1 | 0.1 | −20 | 5 |
| | | Last* | 84 | −0.4 | 2.9 | 4.4 | −20 | 5 |

PCS: LOW: N/A HIGH: N/A

| | | | | | | | | | | | | |
|---|---|---|---|---|---|---|---|---|---|---|---|---|
| WEIGHT | PBO | Week 6 | 112 | −0.3 | 1.9 | 0.0 | −7 | 5 | 2 | (1.8) | 0 | (0.0) |
| | | Month 3 | 78 | −0.4 | 3.3 | 0.0 | −11 | 11 | 7 | (9.0) | 2 | (2.6) |
| | | Month 6 | 43 | −0.4 | 3.9 | 0.0 | −14 | 9 | 4 | (9.3) | 3 | (7.0) |
| | | Last* | 150 | −0.4 | 3.4 | 0.0 | −14 | 23 | 11 | (7.3) | 4 | (2.7) |
| | BX20 | Week 6 | 104 | −0.6 | 2.0 | 0.0 | −7 | 4 | 5 | (4.8) | 0 | (0.0) |
| | | Month 3 | 81 | −0.6 | 2.7 | 0.0 | −9 | 5 | 8 | 99.9 | 0 | (0.0) |
| | | Month 6 | 59 | −0.6 | 3.7 | 0.0 | −13 | 7 | 6 | (10.2) | 4 | (6.8) |
| | | Last* | 143 | −1.0 | 2.9 | 0.0 | −13 | 7 | 13 | (9.1) | 4 | (2.8) |
| | BX30 | Week 6 | 106 | −0.9 | 3.7 | 0.0 | −32 | 4 | 5 | (4.7) | 0 | (0.0) |
| | | Month 3 | 88 | −1.1 | 3.4 | −0.5 | −10 | 6 | 10 | (11.4) | 1 | 1.1 |
| | | Month 6 | 55 | −0.1 | 3.3 | 0.0 | −10, | 8 | 4 | (7.3) | 4 | (7.3) |
| | | Last* | 148 | −1.2 | 3.9 | 0.0 | −32 | 8 | 14 | (9.5) | 4 | (2.7) |

PCS: LOW: Decrease from baseline >= 7%, HIGH: Increase from baseline >= 7%
PCS: LOW: N/A HIGH: N/A
*Last post-Baseline observation

### Time to Deterioration

Clinical Study Four:

The primary efficacy variable was the time to deterioration and the analysis was based on the FAS. The primary efficacy analysis rejected the hypothesis of equal time to deterioration of schizophrenia in the three treatment groups (p=0.008), as shown in table 52. The proportion of patients who deteriorated was 59% in the PBO group, 41% in the BX20 group, and 38% in the BX30 group. The Cox proportional hazards model gave an estimated hazard ratio of 0.66 (BX20) and 0.65 (BX30) relative to PBO; that is, the risk of deterioration was approximately 1.5 times higher for patients in the PBO group than for patients in the BX20 or BX30 groups.

Subsequent pairwise comparisons of each of the BX groups and the PBO group showed that patients in the BX groups had a statistically significantly longer time to deterioration of schizophrenia than patients in the PBO group (BX20. p=0.008 and BX30: p=0.006). The primary efficacy analysis was repeated for the PPS. Since most of the patients in the PPS participated for most of the study, the results were very close to the results of the primary analysis, both for the estimated hazard ratios and the p-values obtained. This illustrates the robustness of the conclusion of the primary efficacy analysis.

TABLE 52
Log-rank Test Analyzing Time to Deterioration - Overall
Test (FAS).

| | | | | Hazard | Cox- |
|---|---|---|---|---|---|
| | | No. of Pts | % | Ratio | Model |
| Treatment | N | Deteriorated | Deteriorated | (Cox) | P-value |
| PBO | 166 | 98 | 59.0 | — | — |
| BX20 | 158 | 64 | 40.5 | 0.656 | — |
| BX30 | 172 | 66 | 38.4 | 0.653 | — |
| All | 496 | 228 | 46.0 | — | 0.008 |

### Lipid Profile

CLINICAL STUDY FOUR: The adjusted mean HDL cholesterol values increased from baseline to Month 6 in all three treatment groups irrespective of fasting/non-fasting condition (PBO: 0.04/0.06 (fasting/nonfasting); BX20: 0.07/0.08; BX30: 0.07/0.08 mmol/L). There were no statistically significant differences between either of the BX groups and the PBO group. The adjusted mean triglycerides values decreased from baseline to Month 6 in all three treatment groups irrespective of fasting/non-fasting condition (PBO: −0.06/−0.22 (fasting/non-fasting); BX20: −0.16/−0.21; BX30: −0.37/−0.03 mmol/L). There were no statistically significant differences between either of the BX groups (except BX30 (fasting)) and the PBO group.

The adjusted mean fasting glucose values increased from baseline to Month 6 in all three treatment groups (PBO: 0.10; BX20: 0.13; BX30: 0.09 mmol/L) and there were no statistically significant differences between either of the BX groups and the PBO group.

Table 53 below summarizes the lipid profile at baseline (mean values) and at Month 6 (mean change from baseline) in fasting and non-fasting patients. The mean total cholesterol and mean LDL calculated decreased from baseline to Month 6 in all groups (except non-fasting PBO patients) irrespective of treatment and fasting/non-fasting condition. The mean VLDL calculated and mean triglycerides decreased from baseline to Month 6 in all groups (except non-fasting BX30 patients) irrespective of treatment and fasting/non-fasting condition. The mean HDL increased from baseline to Month 6 in all groups irrespective of treatment and fasting/nonfasting condition.

TABLE 53
Lipid Profile.

| | | | PBO | | BX20 | | BX30 | |
|---|---|---|---|---|---|---|---|---|
| Variable | Time point | | n. | Conc. | n. | Conc. | n. | Conc. |
| Cholesterol, | Fasting | Baseline | 90 | 4.96 | 81 | 4.82 | 88 | 4.87 |
| total | | Mean change^{a} | 27 | −0.3 | 38 | −0.18 | 37 | −0.22 |
| (mmol/L) | Non-fasting | Baseline | 73 | 4.76 | 77 | 4.69 | 82 | 5.00 |
| | | Mean change^{a} | 17 | 0.07 | 19 | −0.42 | 20 | −0.10 |
| LDL, | Fasting | Baseline | 89 | 2.97 | 77 | 2.88 | 85 | 2.95 |
| calculated^{b} | | Mean change^{a} | 27 | −0.09 | 37 | −0.1 | 37 | −0.12 |
| (mmol/L) | Non-fasting | Baseline | 72 | 2.82 | 76 | 2.75 | 80 | 3.06 |
| | | Mean change^{a} | 17 | 0.05 | 19 | −0.35 | 20 | −0.12 |
| VLDL, calculated | Fasting | Baseline | 90 | 0.71 | 81 | 0.72 | 88 | 0.71 |
| (mmol/L) | | Mean change^{a} | 27 | −0.02 | 38 | −0.03 | 37 | −0.16 |
| | Non-fasting | Baseline | 73 | 0.70 | 77 | 0.73 | 82 | 0.69 |
| | | Mean change^{a} | 17 | −0.06 | 19 | −0.19 | 20 | 0.03 |
| HDL, direct | Fasting | Baseline | 90 | 1.27 | 81 | 1.20 | 88 | 1.18 |
| (mmol/L) | | Mean change^{a} | 27 | 0.09 | 38 | 0.07 | 37 | 0.05 |
| | Non-fasting | Baseline | 73 | 1.23 | 77 | 1.22 | 82 | 1.26 |
| | | Mean change^{a} | 17 | 0.05 | 19 | 0.12 | 20 | 0.08 |
| Triglycerides | Fasting | Baseline | 90 | 1.56 | 81 | 1.67 | 88 | 1.61 |
| (mmol/L) | | Mean change^{a} | 27 | −0.05 | 38 | −0.08 | 37 | −0.40 |
| | Non-fasting | Baseline | 73 | 1.56 | 77 | 1.64 | 82 | 1.53 |
| | | Mean change^{a} | 17 | −0.27 | 19 | −0.41 | 20 | 0.06 |

All values are mean values.
^{a}Mean change from baseline to Month 6
^{b}See also Table 197 for LDL, direct (measured at Baseline in 12 patients (PBO: 2; BX20: 5; BX: 5) and at Month 6 in 1 patient (BX20)).

### Hyperglycemia and Diabetes Mellitus

Hyperglycemia, in some cases extreme and associated with ketoacidosis or hyperosmolar coma or death, has been reported in patients treated with atypical antipsychotics. The incidence of hyperglycemia and diabetes-related adverse events (such as hyperglycemia, elevated blood glucose, impaired glucose tolerance, diabetes mellitus, inadequately controlled diabetes) in patients treated with bifeprunox was 0.5% (5/1050) and in placebo-treated patients was 0.6% (3/469) in six-week placebo-controlled trials. In a 26-week placebo-controlled trial, no patients reported hyperglycemia or diabetes-related adverse events. Assessment of the relationship between atypical antipsychotic use and glucose abnormalities is complicated by the possibility of an increased background risk of diabetes mellitus in patients with schizophrenia and the increasing incidence of diabetes mellitus in the general population. Given these confounders, the relationship between atypical antipsychotic use and hyperglycemia-related adverse events is not completely understood. However, epidemiological studies that did not include bifeprunox suggest an increased risk of treatment-emergent hyperglycemia-related adverse events in patients treated with the atypical antipsychotics included in these studies. Because bifeprunox was not marketed at the time these studies were performed, it is not known if bifeprunox is associated with this increased risk. Precise risk estimates for hyperglycemia-related adverse events in patients treated with atypical antipsychotics are not available.

### PR, OT, QTc, QRS

CLINICAL STUDY ONE: Changes in PR, QT, QTc, QRS intervals and heart rate over time were evaluated by assessing changes in mean values between Baseline and Endpoint. There was very little change in mean values for PR, QTc, or QRS intervals between Baseline and Endpoint in any treatment group. Mean changes for these intervals ranged from approximately −2 msec to 4 msec. There were no trends in mean changes by treatment group. Changes in mean heart rate between Baseline and Endpoint ranged from −1.5 bpm to 0.9 bpm. There were no trends in mean changes by treatment group.

CLINICAL STUDY TWO: Changes in PR, OT, QTc, QRS intervals and heart rate over time were evaluated by assessing changes in mean values between Baseline and Endpoint. There was very little change in mean values for PR, QTc, or QRS intervals between Baseline and Endpoint in any treatment group. Ranges for the mean changes across the treatment groups were: PR=−2.5 msec to 0.2 msec; QT=−2.8 msec to 7 msec, QTc=0.4 msec to 3.9 msec; QRS=−0.4 msec to 1.4 msec. There were no trends in mean changes by treatment group. Changes in mean heart rate between Baseline and Endpoint across the treatment groups ranged approximately from −1.3 bpm to 1.7 bpm. There were no trends in mean changes by treatment group.

CLINICAL STUDY THREE: Changes in PR, QT, QTc, QRS intervals and heart rate over time were evaluated by assessing changes in mean values between Baseline and Endpoint. For these summaries, the Bazett corrected OT interval was presented as OTc. There was very little change in mean values for PR, QTc, or QRS intervals between Baseline and Endpoint in any treatment group. Mean changes for these intervals ranged from approximately 3 msec to 4 msec. There were no trends in mean changes by treatment group. Changes in mean heart rate between Baseline and Endpoint ranged from −1 bpm to 1 bpm. There were no trends in mean changes by treatment group.

### Example 3a

### Pharmacokinetics of Bifeprunox

The objective of this study is to assess the pharmacokinetics (PK) of bifeprunox. The PK of bifeprunox in healthy subjects were investigated based on a pooled analysis of PK parameters from 21 clinical pharmacology studies. The pooled analysis included PK profiles after single and multiple doses to 132 and 399 subjects, respectively, and explored the potential effects of age, gender, body weight, and race. In addition, PK in patients with schizophrenia were investigated using a population PK approach based on samples from 376 patients in phase II studies and 434 patients in phase III studies.

Bifeprunox was rapidly absorbed after oral administration (tₘₐₓ from 1.5 to 2 hours at all dose levels). Bifeprunox multiple-dose PK were dose-proportional in the 20-40 mg/day range. Steady-state mean apparent clearance and apparent volume of distribution were 62.2 L/h and 1300 L, respectively. Bifeprunox was eliminated with a mean plasma steady-state half-life of 14.4 hours. Administration of a 40 mg dose with a standard high-fat meal was associated with a slight delay in tₘₐₓ (1.5 hours) and a small increase in Cₘₐₓ (10%) and AUC (29%). Bifeprunox is approximately 99% bound to serum proteins. Bifeprunox is metabolized by CYP2C9, CYP3A4 and to a lesser extent, CYP2D6. Bifeprunox exposure was increased by co-administration with fluconazole (CYP2C9 inhibitor) and to a minor extent ketoconazole (CYP3A4 inhibitor), but not by coadministration with paroxetine (CYP2D6 inhibitor) and famotidine (a H2-antagonist). Bifeprunox exposure was reduced by co-administration of carbamazepine (CYP3A4 inducer). Co-administration of the narrow therapeutic index compounds warfarin and lithium (see EXAMPLE 3b) with bifeprunox did not affect the PK of these compounds to any relevant extent. In CYP2C9 slow/intermediate metabolizers, higher plasma levels of bifeprunox were observed than in subjects with normal enzyme activity. After a single oral dose of [14C]-labeled bifeprunox, 13% and 74% of the radioactivity was excreted in the urine and feces, respectively. No clinically significant age-, gender-, body weight- or race-related effects on bifeprunox PK were noted. PK in patients with schizophrenia were similar to that seen in healthy subjects.

One conclusion of this study is that bifeprunox is rapidly absorbed after oral administration; the mean elimination half-life is about 14 hours. Multiple-dose PK were dose-proportional in the 2040 mg range. Bifeprunox has a low interaction potential.

### Example 3b

### Pharmacokinetic Interaction of Lithium and Bifeprunox in Healthy Male Subjects

PURPOSE: Bifeprunox, a partial agonist for dopamine D2 and 5-HT1A receptors, is being developed for the treatment of schizophrenia. Because bifeprunox may be used in combination with the mood-stabilizing drug lithium for the treatment of patients with psychoses and mood disorders, the effect of multiple doses of bifeprunox on the pharmacokinetic (PK) profile of lithium was evaluated. Lithium has a narrow therapeutic index that can complicate therapy, and serum levels greater than 1.5 mmol/L carry a greater risk of lithium toxicity than do lower levels.

METHODS: This was a single center, double-blind, randomized, placebo-controlled, parallel-design study in 48 healthy male subjects. All subjects were to receive open-label lithium (450 mg) twice daily on days 1 through 8 and, provided serum levels were stable on days 5 through 7, again on days 9 through 20. Subjects whose serum levels were stable on days 5 through 7 were included in those randomized to receive, in addition to lithium 450 mg twice daily, either placebo or a rising dose of bifeprunox (0.025-40 mg) once daily on days 9 through 17, and placebo or bifeprunox 40 mg on days 18 through 21. Only a single morning dose of lithium 450 mg was administered on day 21. Lithium, steady-state Cₘₐₓ, AUC over the dosing interval (0-t), and renal clearance (CL-R) values were compared between the bifeprunox and placebo groups using ANCOVA with baseline values of lithium measured on day 8 as covariate.

RESULTS: There were small increases in mean Cmax and AUC(0-t) of lithium in the bifeprunox group, but the ratios of the geometric least square means and the 90% confidence intervals (CI) of the two treatments for AUC, Cmax, and CL-R were within the predefined range of 0.80 to 1.25. The treatment ratios and 90% CI for Cmax, AUC(0-t), and CL-R were 1.11 (90% CI: 1.01-1.20), 1.13 (90% CI: 1.06-1.21), and 0.94 (90% CI: 0.87-1.02) respectively. Combined administration of bifeprunox up to 40 mg per day and lithium 450 mg twice daily was well tolerated.

CONCLUSION: There was no clinically relevant effect of co-administration of multiple doses of bifeprunox on lithium steady-state pharmacokinetics. Results suggest that no dosage adjustment of lithium would be required during concomitant administration with bifeprunox.

### Example 4

### Efficacy and Safety of Bifeprunox in Treatment of Patients with Acutely Exacerbated Schizophrenia

OBJECTIVE: To evaluate the efficacy and safety of bifeprunox in the treatment of acutely ill patients with schizophrenia.

METHOD: A 6-Week randomized, placebo-controlled, risperidone-referenced dose-finding study included 589 randomized patients with an acute exacerbation of schizophrenia (DSM-IV-TR). Patients were randomly assigned to bifeprunox 5 mg (n=115), bifeprunox 10 mg (n=120), bifeprunox 20 mg (n=115), placebo (n=119) or risperidone 6 my (n=120). Treatment with all bifeprunox doses were titrated up to target dose, beginning with a dose of 0.125 mg on day 1, 0.25 my on day 2, 0.5 mg on day 3, 1 mg on day 4, 2 mg on day 5, and 5 mg on day 6, 10 mg on day 7 or 20 mg on day 8, while treatment with risperidone was titrated over 3 days. The change in the Positive and Negative Symptom Scale (PANSS) total score, from baseline to endpoint, was the primary outcome measure. Secondary efficacy measures included: PANSS positive, PANSS negative, PANSS general psychopathology (GPP) score, PANSS-derived Brief Psychiatric Rating Scale (BPRS) score, Clinical Global Impressions-Severity of Illness (CGI-S), CGI-Improvement (CGI-I) scores, and responder rates. Safety and tolerability evaluations included extrapyramidal symptoms (EPS), weight gain, lipid profile, and serum prolactin. Risperidone was included for assay sensitivity.

RESULTS: Reduction in the PANSS total score for bifeprunox was statistically significant (P<0.05) compared to placebo at Week 3 and Week 6/endpoint for the 20 mg dose over the treatment period of 6 weeks. The positive effect of bifeprunox 20 mg was also observed on the secondary efficacy measures PANSS positive, negative, GPP subscales, BPRS, and responder rates. Risperidone 6 mg was statistically significant at endpoint compared to placebo for all efficacy measures. The most common adverse events (incidence >5% and twice for placebo) included: dyspepsia, nausea, vomiting, and constipation. A dose relationship was not evident for any of the most frequent adverse events. Bifeprunox was associated with decreased prolactin levels, and rates of EPS that were comparable to placebo. In addition, patients receiving bifeprunox experienced statistically significant (P<0.05) weight decrease, and demonstrated statistically significant improvements in non-fasting triglycerides (P<0.005) and total cholesterol (P<0.005) compared to placebo.

CONCLUSION: In this study, bifeprunox 20 mg was shown to be effective in the treatment of acute schizophrenia. Bifeprunox may have safety advantages, stemming from a decrease in weight, and improvement in the lipid profile.

### Example 5

### Efficacy and Safety of Bifeprunox in Treatment of Patients with Acutely Exacerbated Schizophrenia and Stable Schizophrenia

OBJECTIVE: To examine the metabolic effects of bifeprunox in patients with acute and stable schizophrenia. This pooled analysis evaluated the metabolic effects of bifeprunox as compared to placebo and active reference medications in patients with acute and stable schizophrenia.

METHODS: Metabolic data was compiled from one 6-month and four 6-week studies (i.e., a treatment period) of bifeprunox in patients with stable and acute schizophrenia, respectively. In the 6-month study, patients received bifeprunox (n=331) or placebo (PBO) (n=166). In the 6-week studies, the pooled treatment groups were: bifeprunox (n=1050), PBO (n=469), haloperidol (n=52), risperidone (n=274) and olanzapine (n=150). All studies were randomized, double-blinded and PBO-controlled; an active reference was included in all 6-week studies. Metabolic evaluations included body weight, body mass index (BMI), plasma glucose and lipid profiles. Fasting glucose and lipid values were assessed in one 6-week study and in the 6-month study. Triglyceride:HDL ratio (TG:HDL) was calculated as a marker of insulin resistance. No statistical analyses were performed; only descriptive statistics are presented.

RESULTS: A total of 1,995 patients were randomized to bifeprunox (n=1,050), placebo (n=469), risperidone (n=274), olanzapine (n=150) or haloperidol (n=52) in the 6-week trials. A total of 497 patients received bifeprunox 20 or 30 mg (n=59 and n=172, respectively) or placebo (n=166) in the 6-month study. Tables 54 and 55 show baseline and demographic characteristics in the 6-week trials and 6-month study.

TABLE 54
Baseline and demographic characteristics in 6-week placebo-
controlled studies.

| | BX | PBO | RISP | OLZ | HAL |
|---|---|---|---|---|---|
| Total number of | 1050 | 469 | 274 | 150 | 52 |
| patients | | | | | |
| Age (years) | 39.2 (10.8) | 39.0 (10.3) | 39.4 (9.8) | 39.0 (12.0) | 34.5 (9.0) |
| Mean (SD) | | | | | |
| Sex, n (%) | | | | | |
| Female | 311 (29.6) | 118 (25.2) | 65 (23.7) | 37 (24.7) | 22 (42.3) |
| Male | 739 (70.4) | 351 (74.8) | 209 (76.3) | 113 (75.3) | 30 (57.7) |
| Ethnic origin, n (%) | | | | | |
| Hispanic | 130 (12.4) | 68 (14.5) | 38 (13.9) | 32 (21.3) | 0 |
| Non-Hispanic | 920 (87.6) | 401 (85.5) | 236 (86.1) | 118 (78.7) | 52 (100) |
| Race, n (%) | | | | | |
| American Indian | 11 (1.1) | 3 (0.7) | 5 (1.9) | 0 | 0 |
| Asian | 144 (14.1) | 69 (15.2) | 42 (16.0) | 30 (20.0) | 0 |
| Black | 376 (36.9) | 141 (31.0) | 99 (37.8) | 51 (34.)) | 0 |
| Pacific Islander | 5 (0.5) | 1 (0.2) | 1 (0.4) | 1 (0.7) | 0 |
| White | 481 (47.2) | 237 (52.1) | 112 (42.7) | 68 (45.3) | 52 (100) |
| Other | 3 (0.3) | 4 (0.9) | 3 (1.1) | 0 | 0 |
| Weight (kg) | | | | | |
| Mean (SD) | 80.5 (24.4) | 80.1 (23.2) | 83.0 (24.7) | 79.5 (23.9) | 69.3 (14.6) |
| Median | 77.0 | 77.0 | 82.8 | 78.2 | 69.0 |
| Min-Max | 35-178 | 34-160 | 37-183 | 31-136 | 48-102 |
| BMI (kg/m²) | | | | | |
| Mean (SD) | 27.5 (7.7) | 27.4 (7.3) | 28.4 (7.7) | 27.3 (8.3) | 23.8 (4.7) |
| Median | 25.9 | 26.4 | 27.1 | 26.0 | 23.5 |
| Min-Max | 14-60 | 14-56 | 14-67 | 14-78 | 17-42 |

BX—bifeprunox;
PBO—placebo;
RISP—risperidone;
OLZ—olanzapine;
HAL—haloperidol
Bifeprunox data is from all treatment arms, including fixed doses ranging from 1 mg to 40 mg

TABLE 55
Baseline and demographic characteristics in 6-month
placebo-controlled study.

| | BX | PBO |
|---|---|---|
| Total number of patients | 331 | 166 |
| Mean Age (years), SD | 40.8 (11.0) | 39.3 (11.0) |
| Sex, n (%) | | |
| Female | 183 (55.3) | 86 (51.8) |
| Male | 148 (44.7) | 80 (48.2) |
| Race, n (%) | | |
| American Indian | 0 | 0 |
| Asian | 0 | 0 |
| Black | 0 | 1 (0.6) |
| Pacific Islander | 0 | 0 |
| White | 331 (100) | 165 (99.4) |
| Other | 0 | 0 |
| Weight (kg) | | |
| Mean (SD) | 71.3 (14.6) | 71.3 (14.5) |
| Median | 69 | 70 |
| Min-Max | 38-126 | 42-117 |
| BMI (kg/m²) | | |
| Mean (SD) | 24.8 (4.4) | 25.2 (4.8) |
| Median | 24 | 24.2 |
| Min-Max | 14-44 | 17-42 |

BX—bifeprunox;
PBO—placebo
Bifeprunox data is from all treatment arms, including fixed doses of 20 mg and 30 mg

Weight

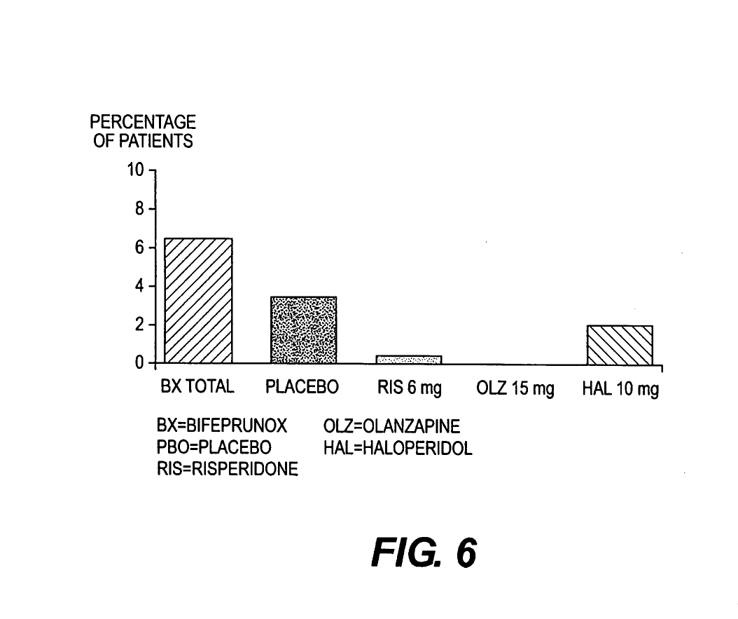

The following results were obtained from the data in the 6-week studies. The mean weight of bifeprunox-treated patients decreased 0.8 kg compared with a 0.1-kg decrease among placebo- and haloperidol-treated patients. Conversely, mean weight increased 1.7 kg and 2.4 kg with risperidone and olanzapine at endpoint, respectively. A ≧7% or ≦7% change in weight from baseline to any post-baseline time point was considered clinically significant; and clinically significant decreases in weight were more common with bifeprunox than with placebo, risperidone, olanzapine and haloperidol (FIG. 6).

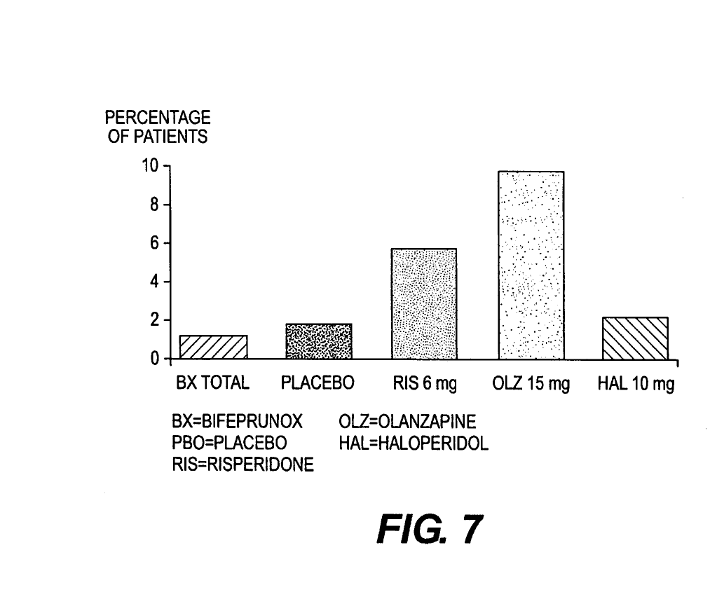

Clinically significant weight increases were less frequent among bifeprunox-treated patients than among placebo-, risperidone-, olanzapine-, and haloperidol-treated patients. No dose relationship was observed in bifeprunox-treated patients (FIG. 7).

Mean decreases in weight occurred among bifeprunox-treated patients regardless of nausea and vomiting (−1.7 kg with nausea or vomiting versus −0.4 kg without nausea or vomiting). Placebo-treated patients with nausea or vomiting had a change of −0.5 kg versus a 0-kg change (no mean change) without nausea or vomiting at endpoint. Mean weight changes across all BMI categories are shown in Table 58.

TABLE 56
Change in weight by baseline BMI at endpoint in 6-week placebo
controlled trials

| | Baseline BMI (kg/m²) | | | | |
|---|---|---|---|---|---|
| Treatment Change | <18.5 | 18.5-25 | >25-30 | >30 | All |
| BX total doses | 18 | 332 | 217 | 277 | 907 |
| (n = 1050) | | | | | |
| Mean (SD) change | 0.0 (1.7) | −0.4 (2.6) | −0.9 (3.2) | −1.4 (3.9) | −0. (3.2) |
| from baseline (kg) | | | | | |
| ≧7% weight increase, | 3 (3.7%) | 8 (2.4%) | 4 (1.8%) | 4 (1.4%) | 19 (2.1%) |
| n (%) | | | | | |
| ≧7% weight decrease, | 3 (3.7%) | 18 (5.4%) | 19 (8.8%) | 16 (5.8%) | 56 (6.2%) |
| n (%) | | | | | |
| PBO (n = 469) | 39 | 154 | 97 | 122 | 412 |
| Mean (SD) change | 0.2 (1.3) | 0.3 (3.6) | −0.4 (3.0) | −0.4 (3.1) | −0.1 (3.1) |
| from baseline (kg) | | | | | |
| ≧7% weight increase, | 1 (2.6%) | 7 (4.5%) | 4 (4.1%) | 1 (0.8%) | 13 (3.2%) |
| n (%) | | | | | |
| ≧7% weight decrease, | 0 | 5 (3.2%) | 7 (7.2%) | 2 (1.6%) | 14 (3.4%) |
| n (%) | | | | | |
| RISP 6 mg (n = 274) | 10 | 82 | 60 | 82 | 234 |
| Mean (SD) change | 0.9 (2.3) | 1.5 (2.2) | 2.1 (3.2) | 1.6 (4.2) | 1.7 (3.3) |
| from baseline (kg) | | | | | |
| ≧7% weight increase, | 2 (20.0%) | 6 (7.3%) | 8 (13.3%) | 7 (8.5%) | 23 (9.8%) |
| n (%) | | | | | |
| ≧7% weight decrease, | 0 | 0 | 0 | 1 (1.2%) | 1 (0.4%) |
| n (%) | | | | | |
| OLZ 15 mg (n = 150) | 16 | 44 | 38 | 41 | 139 |
| Mean (SD) change | 1.0 (1.9) | 2.1 (2.7) | 2.3 (2.5) | 3.3 (4.0) | 2.4 (3.1) |
| from baseline (kg) | | | | | |
| ≧7% weight increase, | 3 (18.8%) | 10 (22.7%) | 5 (13.2%) | 6 (14.6%) | 24 (17.3%) |
| n (%) | | | | | |
| ≧7% weight decrease, | 0 | 0 | 0 | 0 | 0 |
| n (%) | | | | | |
| HAL 10 mg (n = 52) | 5 | 28 | 12 | 5 | 50 |
| Mean (SD) change | 1.4 (1.3) | −0.0 (2.2) | −1.1 (1.8) | 0.4 (2.1) | −0.1 (2.1) |
| from baseline (kg) | | | | | |
| ≧7% weight increase, | 0 | 2 (7.1%) | 0 | 0 | 2 (4.0%) |
| n (%) | | | | | |
| ≧7% weight decrease, | 0 | 1 (3.6%) | 0 | 0 | 1 (2.0%) |
| n (%) | | | | | |

BX—bifeprunox;
PBO—placebo;
RISP—risperidone;
OLZ—olanzapine;
HAL—haloperidol

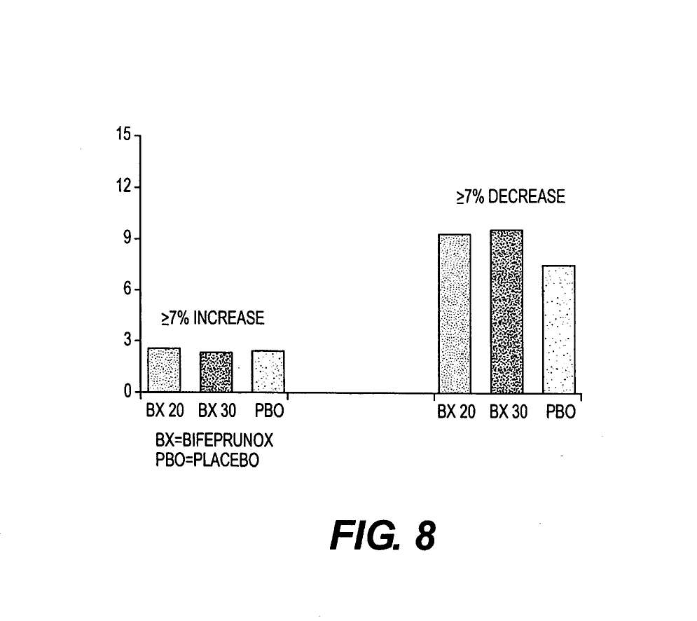

The following results were obtained from the data in the 6-month study. At the final evaluation (LOCF), bifeprunox-treated patients had a mean weight change of −1.1 kg versus −0.5 kg in placebo-treated patients. Changes in weight considered clinically significant were observed with bifeprunox, but no difference between bifeprunox and placebo in terms of weight increases from baseline were observed. Clinically significant weight decreases were more frequent with bifeprunox than placebo (FIG. 8). Mean decreases in weight were observed among bifeprunox-treated patients regardless of the incidence of nausea or vomiting (−1.4 kg with nausea or vomiting versus −1.0 kg without nausea or vomiting). Placebo-treated patients had a mean decrease in weight of 1.9 kg with, versus 0.4 kg without, nausea and vomiting. Mean weight decreases were observed in bifeprunox-treated patients across all BMI categories, and were most pronounced for those with the highest BMI (Table 57).

TABLE 57
Change in weight by baseline BMI at endpoint in 6-month study.

| | Baseline BMI (kg/m²) | | | | |
|---|---|---|---|---|---|
| Treatment Change | <18.5 | 18.5-25 | >25-30 | >30 | All |
| BX total doses | 13 | 183 | 85 | 39 | 320 |
| Mean (SD) change | −0.8 (2.6) | −0.9 (2.9) | −0.9 (2.7) | −3.0 (5.6) | −1.1 (3.3) |
| from baseline (kg) | | | | | |
| ≧7% weight increase, | 0 | 6 (3.3%) | 2 (2.4%) | 0 | 8 (2.5%) |
| n (%) | | | | | |
| ≧7% weight decrease, | 3 (23.1%) | 16 (8.7%) | 4 (4.7%) | 5 (12.8%) | 28 (8.8%) |
| n (%) | | | | | |
| PBO (n = 469) | 4 | 86 | 50 | 23 | 163 |
| Mean (SD) change | −1.3 (2.6) | −0.8 (3.2) | 0.3 (3.9) | −0.7 (2.2) | −0.5 (3.3) |
| from baseline (kg) | | | | | |
| ≧7% weight increase, | 0 | 2 (2.3%) | 2 (4.0%) | 0 | 4 (2.5%) |
| n (%) | | | | | |
| ≧7% weight decrease, | 1 (25.0%) | 10 (11.6%) | 0 | 0 | 11 (6.7%) |
| n (%) | | | | | |

BX—bifeprunox;
PBO—placebo

Lipids

The following results were obtained from the data in the 6-week studies. The greatest decreases in non-fasting total cholesterol and triglyceride at endpoint were seen with bifeprunox (Table 58).

TABLE 58
Changes in non-fasting lipid profile tests at endpoint in 6-week
studies.

| | | | | |
|---|---|---|---|---|
| BX | | RISP | OLZ | HAL |
| total | PBO | 6 mg | 15 mg | 10 mg |

Total cholesterol, mg/dL

| | | | | | |
|---|---|---|---|---|---|
| Baseline, n | 831 | 363 | 272 | 41 | 52 |
| Mean (SD) | 192.0 (1.2) | 193.4 (1.2) | 192.9 (1.1) | 186.9 (1.3) | 191.3 (1.1) |
| Endpoint, n | 743 | 318 | 235 | 41 | 49 |
| Mean change (SD) | −12.9 (0.8) | −10.7 (0.9) | −2.6 (0.8) | −0.7 (1.0) | 0.1 (0.7) |

Triglycerides, mg/dL

| | | | | | |
|---|---|---|---|---|---|
| Baseline, n | 831 | 363 | 272 | 41 | 52 |
| Mean (SD) | 185.7 (1.5) | 198.2 (1.6) | 195.1 (1.4) | 169.2 (1.1) | 129.3 (0.8) |
| Endpoint, n | 743 | 319 | 235 | 41 | 49 |
| Mean change (SD) | −38.7 (1.3) | −26.7 (1.1) | −7.4 (1.4) | 24.6 (1.3) | −2.7 (0.6) |

BX—bifeprunox;
PBO—placebo;
RISP—risperidone;
OLZ—olanzapine;
HAL—haloperidol

In the 6-week studies, fasting total cholesterol, triglyceride and LDL data were similar to non-fasting. Table 59 shows fasting values in studies that measured this endpoint.

TABLE 59
Changes in fasting lipid profile tests at
endpoint in 6-week studies.

| | | |
|---|---|---|
| BX total | PBO | OLZ 15 mg |

Total cholesterol, mg/dL

| | | | |
|---|---|---|---|
| Baseline, n | 207 | 99 | 102 |
| Mean (SD) | 195.9 (53.2) | 201.6 (46.7) | 198.8 (58.8) |
| Endpoint, n | 130 | 66 | 74 |
| Mean change (SD) | −15.5 (32.9) | −12.4 (30.4) | −4.0 (37.3) |
| Percentage mean change | −7.9 | −6.1 | −2.0 |

Triglycerides, mg/dL

| | | | |
|---|---|---|---|
| Baseline, n | 207 | 99 | 102 |
| Mean (SD) | 161.2 (97.0) | 172.8 (100.7) | 175.2 (113.3) |
| Endpoint, n | 130 | 66 | 74 |
| Mean change (SD) | −38.9 (73.5) | −33.7 (136.4) | −9.2 (90.4) |
| Percentage mean change | −24.2 | −19.5 | −5.3 |

TFasting LDL, mg/dL

| | | | |
|---|---|---|---|
| Baseline, n | 199 | 94 | 98 |
| Mean (SD) | 16.8 (45.9) | 124.2 (405) | 114.9 (46.1) |
| Endpoint, n | 124 | 60 | 69 |
| Mean change (SD) | −8.6 (29.0) | −9.6 (27.3) | −0.3 (28.6) |
| Percentage mean change | −7.3 | −7.7 | −0.3 |

TFasting HDL, mg/dL

| | | | |
|---|---|---|---|
| Baseline, n | 207 | 99 | 102 |
| Mean (SD) | 48.1 (14.8) | 47.1 (15.8) | 47.8 (15.3) |
| Endpoint, n | 130 | 66 | 74 |
| Mean change (SD) | −0.5 (10.7) | −1.0 (16.8) | −1.4 (9.2) |
| Percentage mean change | −1.0 | −2.2 | −2.9 |

Triglyceride:HDL ratio

| | | | |
|---|---|---|---|
| Baseline, n | 207 | 99 | 102 |
| Mean (SD) | 1.7 (1.4) | 1.8 (1.3) | 1.8 (1.4) |
| Endpoint, n | 130 | 66 | 74 |
| Mean change (SD) | −0.4 (0.9) | −0.3 (1.1) | −0.0 (1.2) |
| Percentage mean change | −23.8 | −17.0 | −1.5 |

BX—bifeprunox;
PBO—placebo;
OLZ—olanzapine

The following results were obtained from the data in the 6-month study. Total cholesterol and triglycerides were reduced to a greater extent with bifeprunox than with placebo. In the 6-month study, bifeprunox was associated with a greater reduction in LDL cholesterol, a modest increases in HDL cholesterol, and a larger decrease in the TG:HDL ratio versus placebo. Data from the fasting subset of patients are presented in Table 60.

TABLE 60
Changes in fasting lipid profile tests at
endpoint in 6-month study.

| | |
|---|---|
| BX total | PBO |

Total cholesterol, mg/dL

| | | |
|---|---|---|
| Baseline, n | 170 | 90 |
| Mean (SD) | 187.3 (41.8) | 190.5 (38.3) |
| Endpoint, n | 416 | 74 |
| Mean change (SD) | −11.6 (29.7) | −3.1 (28.5) |
| Percentage mean change | −6.4 | −1.6 |

Triglycerides, mg/dL

| | | |
|---|---|---|
| Baseline, n | 170 | 90 |
| Mean (SD) | 144.9 (89.9) | 137.7 (69.1) |
| Endpoint, n | 147 | 74 |
| Mean change (SD) | −28.0 (76.0) | −4.2 (63.2) |
| Percentage mean change | −19.4 | −3.0 |

Fasting LDL, mg/dL

| | | |
|---|---|---|
| Baseline, n | 163 | 89 |
| Mean (SD) | 112.9 (35.9) | 114.0 (32.7) |
| Endpoint, n | 143 | 73 |
| Mean change (SD) | −9.7 (27.6) | −3 1 (23.5) |
| Percentage mean change | −8.6 | −2.7 |

Fasting HDL, mg/dL

| | | |
|---|---|---|
| Baseline, n | 170 | 90 |
| Mean (SD) | 46.0 (11.7) | 49.0 (14.1) |
| Endpoint, n | 146 | 74 |
| Mean change (SD) | 2.9 (9.3) | 1.2 (12.5) |
| Percentage mean change | 6.2 | 2.4 |

Triglyceride:HDL ratio

| | | |
|---|---|---|
| Baseline, n | 170 | 90 |
| Mean (SD) | 1.5 (1.1) | 1.4 (1.0) |
| Endpoint, n | 146 | 74 |
| Mean change (SD) | −0.4 (1.0) | −0.1 (0.8) |
| Percentage mean change | −23.0 | −3.6 |

BX—bifeprunox;
PBO—placebo

Glucose

Mean fasting glucose values decreased by 1.6% (−1.44 mg/dL) among bifeprunox-treated patients, compared to a 5.6% (+5.05 mg/dL) increase in placebo-treated patients in the 6-week studies at endpoint. Mean fasting glucose values were slightly increased at endpoint in the bifeprunox group in the 6-month study (+4.32 mg/dL) and the placebo group (+0.90 mg/dL). At endpoint, non-fasting glucose values increased +1.16 mg/dL in the bifeprunox total group but were unchanged in the placebo group.

Overall, in the 6-month study, mean weight decreases occurred with bifeprunox (−1.1 kg) and PBO (−0.5 kg) treatment. Similar reductions were noted in 6-week studies (bifeprunox, −0.8 kg; PBO, −0.1 kg). Olanzapine (+2.4 kg) and risperidone (+1.7 kg) groups experienced weight increases. Mean weight loss occurred in patients receiving bifeprunox in all but the lowest BMI group (<18.5 kg/m2), with the greatest weight loss experienced by obese patients (>30 kg/m2) in 6-week studies. A minor reduction in mean fasting glucose versus PBO was noted in one 6-week study; minimal glucose changes from baseline in PBO and bifeprunox groups were observed in 6 week and 6 month tests. Total cholesterol (TC) was decreased in the 6-month (bifeprunox, 6%; −0.31 mmol/L vs. PBO, 2%; −0.08 mmol/L) and 6-week studies (bifeprunox, 8%; −0.40 mmol/L vs. PBO, 6%; −0.32 mmol/L). Over 6-months, TG values decreased in the bifeprunox (19%; −0.32 mmol/L) and PBO groups (3%; −0.05 mmol/L). Additionally, a 23% decrease in TG:HDL was noted in patients receiving bifeprunox in the 6-month study, compared to a 4% decrease in the PBO group. Similar decreases in TG (24%, −0.44 mmol/L) and TG:HDL were observed in 6 week studies; the PBO group had comparable reductions in TG (20%; −0.38 mmol/L) and TG:HDL (17%).

What is claimed is:

1. A composition for treating schizophrenia, comprising
a dose of at least one bifeprunox compound, the dose ranging from about 20 mg to about 30 mg, wherein, over a treatment period, the composition is effective to achieve:
(1) treatment of at least one symptom of schizophrenia; and
(2) either
(a) an absence of at least one side-effect associated with schizophrenia therapy with a first generation atypical antipsychotic; or
(b) a reduced presence of at least one side-effect associated with schizophrenia therapy with a first generation atypical antipsychotic;
in a patient administered the composition

2. The composition according to claim 1, wherein the dose comprises a once-daily dose.

3. The composition according to claim 1, wherein the composition is for long-term treatment of a patient with schizophrenia.

4. The composition according to claim 1, wherein the composition is for maintaining clinical stability in a patient with schizophrenia.

5. The composition according to claim 4, wherein the dose comprises about 20 mg of the at least one bifeprunox compound.

6. The composition according to claim 4, wherein the dose comprises about 30 mg of the at least one bifeprunox compound.

7. The composition according to claim 1, wherein the composition is for treatment of a patient with acutely exacerbated schizophrenia.

8. The composition according to claim 7, wherein the dose comprises about 20 mg of the at least one bifeprunox compound.

9. The composition according to claim 7, wherein the dose comprises about 30 mg of the at least one bifeprunox compound.

10. The composition according to claim 1, wherein the at least one bifeprunox compound comprises bifeprunox mesylate.

11. The composition according to claim 10, wherein the at least one bifeprunox compound comprises the alpha polymorph of bifeprunox mesylate.

12. The composition according to claim 1, further comprising at least one pharmaceutically acceptable excipient.

13. The composition according to claim 1, wherein the treatment period comprises at least six weeks.

14. The composition according to claim 13, wherein the treatment period comprises at least six months.

15. The composition according to claim 1, wherein the at least one side-effect associated with schizophrenia therapy with a first generation atypical antipsychotic is chosen from weight gain, disorders of triglyceride levels and total cholesterol, hyperglycemia, diabetes-related adverse events, and combinations thereof.

16. A composition for treating schizophrenia and improving safety or tolerability over at least one side-effect associated with schizophrenia therapy with at least one first generation atypical antipsychotic, comprising:
a dose of at least one bifeprunox compound, the dose ranging from about 20 mg to about 30 mg, wherein over a treatment period, the composition is effective to achieve:
(1) treatment of at least one symptom of schizophrenia; and
(2) either
(a) an absence of the at least one side effect of the at least one first generation atypical antipsychotic; or
(b) a reduced presence of the at least one side-effect of the at least one first generation atypical antipsychotic,
in a patient administered the composition.

17. A kit for the treatment of a patient with at least one condition chosen from psychoses and mood disorders comprising a first composition comprising a therapeutically effective dose of at least one bifeprunox compound and a second composition comprising a therapeutically effective amount of lithium.

18. The kit according to claim 17, wherein the at least one bifeprunox compound comprises bifeprunox mesylate.

19. The kit according to claim 18, wherein the bifeprunox mesylate comprises the alpha polymorph of bifeprunox mesylate.

20. The kit according to claim 17, wherein the dose of the at least one bifeprunox compound ranges from about 20 mg to about 30 mg.

21. The kit according to claim 17, wherein the schizophrenia is chosen from stable schizophrenia and acutely exacerbated schizophrenia.

22. The kit according to claim 17, wherein the dose of the first composition comprises a once-daily dose.

23. A kit for the treatment of a patient with at least one condition chosen from schizophrenia and depression comprising a first composition comprising a therapeutically effective dose of at least one bifeprunox compound and a second composition comprising a therapeutically effective amount of at least one antidepressant.

24. The kit according to claim 23, wherein the at least one antidepressant comprises paroxetine.

25. The kit according to claim 23, wherein the at least one bifeprunox compound comprises bifeprunox mesylate.

26. The kit according to claim 25, wherein the bifeprunox mesylate comprises the alpha polymorph of bifeprunox mesylate.

27. The kit according to claim 23, wherein the dose of the at least one bifeprunox compound ranges from about 20 mg to about 30 mg.

28. The kit according to claim 23, wherein the schizophrenia is chosen from stable schizophrenia and acutely exacerbated schizophrenia.

29. The kit according to claim 23, wherein the dose of the first composition comprises a once-daily dose.

30. A kit for the treatment of a patient with schizophrenia comprising a first composition comprising a therapeutically effective dose of at least one bifeprunox compound and a second composition comprising a therapeutically effective dose of at least one compound chosen from CYP2C9 inhibitors and CYP3A4 inhibitors.

31. The kit according to claim 30, wherein the at least one compound chosen from CYP2C9 inhibitors and CYP3A4 inhibitors is chosen from fluconazole and ketoconazole.

32. The kit according to claim 30, wherein the at least one bifeprunox compound comprises bifeprunox mesylate.

33. The kit according to claim 32, wherein the bifeprunox mesylate comprises the alpha polymorph of bifeprunox mesylate.

34. The kit according to claim 30, wherein the dose of the at least one bifeprunox compound ranges from about 20 mg to about 30 mg.

35. The kit according to claim 30, wherein the schizophrenia is chosen from stable schizophrenia and acutely exacerbated schizophrenia.

36. The kit according to claim 30, wherein the dose of the first composition comprises a once-daily dose.

37. A method for treating schizophrenia in a patient in need thereof comprising:
co-administering to the patient a composition comprising a therapeutically effective dose of at least one bifeprunox compound and a therapeutically effective dose of at least one compound chosen from CYP2C9 inhibitors and CYP3A4 inhibitors, over a treatment period.

38. The method according to claim 37, wherein the at least one compound chosen from CYP2C9 inhibitors and CYP3A4 inhibitors is chosen from fluconazole and ketoconazole.

39. The method according to claim 37, wherein the schizophrenia is chosen from stable schizophrenia and acutely exacerbated schizophrenia.

40. The method according to claim 37, wherein the dose of the at least one bifeprunox compound ranges from about 20 mg to about 30 mg.

41. The method according to claim 37, wherein the dose of the at least one bifeprunox compound comprises a once-daily dose.

42. A method for preventing the deterioration of schizophrenia in a patient in need thereof comprising:
administering to the patient a composition comprising a dose of at least one bifeprunox compound ranging from about 20 mg to about 30 mg over a treatment period, wherein the dose of at least one bifeprunox compound prevents deterioration of schizophrenia in the patient, from a baseline measurement before administration.

43. The method according to claim 42, wherein deterioration is determined by a measurement chosen from at least one of: Clinical-Global Impression-Global Improvement (CGI-I), Positive and Negative Syndrome Scale (PANSS) total score, PANSS Positive subscale score and PANSS Negative subscale score.

44. The method according to claim 42, wherein the dose comprises a once-daily dose.

45. A method for reducing a patient's PANSS total score during treatment of schizophrenia, comprising:
administering to the patient a composition comprising a dose of at least one bifeprunox compound ranging from about 20 mg to about 30 mg over a treatment period, wherein the composition administered to the patient results in a reduction of the patient's PANSS total score, from a baseline measurement before administration.

46. The method according to claim 45, wherein the dose comprises a once-daily dose.

47. A method for reducing a patient's triglyceride levels during treatment of schizophrenia, comprising:
administering to the patient a composition comprising a dose of at least one bifeprunox compound ranging from about 20 mg to about 30 mg over a treatment period, wherein the patient's triglyceride levels are reduced, when compared with a baseline measurement before administration.

48. The method according to claim 47, wherein the dose comprises a once-daily dose.

49. A method for treating an acutely exacerbated schizophrenic patient in need of treatment comprising:
administering to the patient a composition comprising a dose of at least one bifeprunox compound ranging from about 20 mg to about 30 mg over a treatment period, wherein the composition is effective to improve at least one efficacy measurement in the patient, when compared with a baseline measurement taken before administration.

50. The method according to claim 49, wherein the at least one efficacy measurement is chosen from PANSS total score, PANSS positive, PANSS negative, GPP subscale, BPRS, and responder rates.

51. The method according to claim 49, wherein the dose comprises a once-daily dose.

52. A method for treating schizophrenia in a patient in need thereof comprising:
administering to the patient a composition comprising a dose of at least one bifeprunox compound ranging from about 20 mg to about 30 mg over a treatment period, wherein the composition is effective to achieve a reduction in at least one symptom of schizophrenia in the patient, from a baseline measurement before administration.

53. The method according to claim 52, wherein the treatment period comprises at least six weeks.

54. The method according to claim 53, wherein the treatment period comprises at least a six months.

55. The method according to claim 52, wherein schizophrenia is chosen from stable schizophrenia and acutely exacerbated schizophrenia.

56. The method according to claim 52, wherein the treatment comprises long-term treatment of schizophrenia.

57. The method according to claim 52, wherein the dose comprises a once-daily dose.

58. A method for treating schizophrenia in a patient in need thereof comprising:
administering to the patient a composition comprising a dose of at least one bifeprunox compound ranging from about 20 mg to about 30 my over a treatment period, wherein the composition is effective to achieve an improvement in at least one symptom of schizophrenia in the patient, from a baseline measurement before administration.

59. The method according to claim 58, wherein the dose comprises a once-daily dose.

60. A method for treating at least one condition chosen from psychoses and mood disorders in a patient in need thereof comprising:
co-administering to the patient a first composition comprising a therapeutically effective dose of at least one compound of bifeprunox, and a second composition comprising a therapeutically effective amount of lithium.

61. The method according to claim 60, wherein the dose of the at least bifeprunox compound ranges from about 1 mg to about 40 mg.

62. The method according to claim 60, wherein the dose comprises a once-daily dose.

63. A method for treating at least one condition chosen from schizophrenia and depression in a patient in need thereof comprising:
co-administering to the patient a first composition comprising a therapeutically effective dose of at least one compound of bifeprunox, and a second composition comprising a therapeutically effective amount of at least one antidepressant over a treatment period.

64. The method according to claim 63, wherein the at least one antidepressant comprises paroxetine.

65. The method according to claim 63, wherein the dose of the at least one bifeprunox compound ranges from about 1 mg to about 40 mg.

66. The method according to claim 65, wherein the dose of the at least one bifeprunox compound ranges from about 20 mg to about 30 mg.

67. The method according to claim 63, wherein the dose comprises a once-daily dose.

68. A method for treating schizophrenia in a patient in need thereof comprising:
administering to the patient a composition comprising: a dose of at least one bifeprunox compound, the dose ranging from about 20 mg to about 30 mg, wherein, over a treatment period, the composition is effective to achieve:
(1) treatment of at least one symptom of schizophrenia; and
(2) either: (a) an absence of at least one side-effect associated with schizophrenia therapy with a first generation atypical antipsychotic; or (b) a reduced presence of at least one side-effect associated with schizophrenia therapy with a first generation atypical antipsychotic; in a patient administered the composition.

69. The method according to claim 68, wherein the at least one side-effect associated with schizophrenia therapy with a first generation atypical antipsychotic is chosen from weight gain, disorders of triglyceride levels and total cholesterol, hyperglycemia, diabetes-related adverse events, and combinations thereof.

70. The method according to claim 68, wherein the dose comprises a once-daily dose.

71. A method for treating schizophrenia in a patient in need thereof comprising:
administering to the patient a composition comprising a dose of at least one bifeprunox compound ranging from about 20 mg to about 30 mg over a treatment period, wherein the administered composition results in at least two measurements associated with a favorable metabolic profile chosen from no weight gain, reduction in weight gain, no increase in prolactin, reduction in triglyceride level, reduction in cholesterol, no glucose dysregulation, and no QTc prolongation, compared with baseline measurements before administration.

72. The method according to claim 71, wherein the dose comprises a once-daily dose.

73. A method for treating schizophrenia in a patient in need thereof comprising:
administering to the patient a composition comprising a therapeutically effective dose of at least one bifeprunox compound, wherein administration over a treatment period results in a maintenance or a reduction in weight of the patient, in comparison to a baseline measurement before administration.

74. The method according to claim 73, wherein the reduction in weight comprises a clinically significant decrease.

75. The method according to claim 73, wherein the dose of the at least one bifeprunox compound ranges from about 1 mg to about 40 mg daily.

76. The method according to claim 75, wherein the dose of the at least one bifeprunox compound ranges from about 20 mg to about 30 mg.

77. The method according to claim 73, wherein the patient has a BMI>18.5 kg/m², before administration.

78. The method according to claim 77, wherein the patient has a BMI>25 kg/m², before administration.

79. The method according to claim 78, wherein the patient has a BMI>30 kg/m², before administration.

80. The method according to claim 73, wherein the treatment period comprises at least 6 weeks.

81. The method according to claim 80, wherein the treatment period comprises at least 6 months.

82. The method according to claim 73, wherein the at least one bifeprunox compound comprises biteprunox mesylate.

83. The method according to claim 82, wherein the at least one bifeprunox compound comprises the alpha polymorph of bifeprunox mesylate.

84. The method according to claim 73, wherein the schizophrenia is chosen from stable schizophrenia and acutely exacerbated schizophrenia.

85. The method according to claim 73, wherein administration over the treatment period results in a maintenance or a reduction in total cholesterol, in comparison to a baseline measurement before administration.

86. The method according to claim 73, wherein administration over the treatment period results in a maintenance or a reduction in prolactin, in comparison to a baseline measurement.

87. The method according to claim 73, wherein the dose comprises a once-daily dose.

88. A method for treating schizophrenia in a patient in need thereof comprising:
administering to the patient a composition comprising a dose of at least one bifeprunox compound ranging from about 20 mg to about 30 mg over a treatment period, wherein administration over the treatment period avoids an increase in weight in the patient, in comparison to a baseline measurement before administration.

89. The method according to claim 88, wherein the avoided increase in weight comprises a clinically significant increase.

90. The method according to claim 88, wherein the treatment period comprises at least 6 weeks.

91. The method according to claim 90, wherein the treatment period comprises at least 6 months.

92. The method according to claim 88, wherein the at least one bifeprunox compound comprises bifeprunox mesylate.

93. The method according to claim 92, wherein the at least one bifeprunox compound comprises the alpha polymorph of bifeprunox mesylate.

94. The method according to claim 88, wherein the schizophrenia is chosen from stable schizophrenia and acutely exacerbated schizophrenia.

95. The method according to claim 88, wherein the dose comprises a once-daily dose.
